SEQUENCE LISTING (15 sequences)

SEQ ID NO: 1 - DNA, 2041 bp, Homo sapiens
atgtctcccgcccggctccggccccgactgcacttctgcctggtcctgttgctgctgctggtggtgccggcggcatggggctgcgggccgggtcgggtggtgggcagccgccggcgaccgccacgcaaactcgtgccgctcgcctacaagcagttcagccccaatgtgcccgagaagaccctgggcgccagcggacgctatgaaggcaagatcgctcgcagctccgagcgcttcaaggagctcacccccaattacaatccagacatcatcttcaaggacgaggagaacacaggcgccgaccgcctcatgacccagcgctgcaaggaccgcctgaactcgctggctatctcggtgatgaaccagtggcccggtgtgaagctgcgggtgaccgagggctgggacgaggacggccaccactcagaggagtccctgcattatgagggccgcgcggtggacatcaccacatcagaccgcgaccgcaataagtatggactgctggcgcgcttggcagtggaggccggctttgactgggtgtattacgagtcaaaggcccacgtgcattgctccgtcaagtccgagcactcggccgcagccaagacgggcggctgcttccctgccggagcccaggtacgcctggagagtggggcgcgtgtggccttgtcagccgtgaggccgggagaccgtgtgctggccatgggggaggatgggagccccaccttcagcgatgtgctcattttcctggaccgcgagcctcacaggctgagagccttccaggtcatcgagactcaggaccccccacgccgcctggcactcacacccgctcacctgctctttacggctgacaatcacacggagccggcagcccgcttccgggccacatttgccagccacgtgcagcctggccagtacgtgctggtggctggggtgccaggcctgcagcctgcccgcgtggcagctgtctctacacacgtggccctcggggcctacgccccgctcacaaagcatgggacactggtggtggaggatgtggtggcatcctgcttcgcggccgtggctgaccaccacctggctcagttggccttctggcccctgagactctttcacagcttggcatggggcagctggaccccgggggagggtgtgcattggtacccccagctgctctaccgcctggggcgtctcctgctagaagagggcagcttccacccactgggcatgtccggggcagggagctgaaaggactccaccgctgccctcctggaactgctgtactgggtccagaagcctctcagccaggagggagctggccctggaagggacctgagctgggggacactggctcctgccatctcctctgccatgaagatacaccattgagacttgactgggcaacaccagcgtcccccacccccgtcgtggtgtagtcatagagctgcaagctgagctggcgaggggatggttgttgacccctctctcctagagaccttgaggctggcacggcgactcccaactcagcctgctctcactacgagttttcatactctgcctcccccattggggagggcccattccatccatcttaggcccctttgggtgggcttgcgcctcagttgatgctgctaaattccctgggagccagcatggatctggctggacccgatgctgtccagaactgggaaggccacaggggtggggcagccatcccggccattctgaggtatgacattcctccccggccacactcctcaagacacatccagagactgttgctgtctgtgggcagagttctgtgttctggccaatgtgaccgtagtgccggggactgggggaggtgggttggatgtgcttgccacccccccggctaagctcccccttctgctgaaccatgatccccaccccctccgccggtcagtctcccataccttatttattggagtggagggggaagcccatgggagaattttggggatgttttggtcttttcttccttttgtaataaaaattatttaagttgttagaaaaaaaaaaaaaaaaaaaaaaa

SEQ ID NO: 2 - protein, 411 aa, Homo sapiens
MSPARLRPRLHFCLVLLLLLVVPAAWGCGPGRVVGSRRRPPRKLVPLAYKQFSPNVPEKTLGASGRYEGKIARSSERFKELTPNYNPDIIFKDEENTGADRLMTQRCKDRLNSLAISVMNQWPGVKLRVTEGWDEDGHHSEESLHYEGRAVDITTSDRDRNKYGLLARLAVEAGFDWVYYESKAHVHCSVKSEHSAAAKTGGCFPAGAQVRLESGARVALSAVRPGDRVLAMGEDGSPTFSDVLIFLDREPHRLRAFQVIETQDPPRRLALTPAHLLFTADNHTEPAARFRATFASHVQPGQYVLVAGVPGLQPARVAAVSTHVALGAYAPLTKHGTLVVEDVVASCFAAVADHHLAQLAFWPLRLFHSLAWGSWTPGEGVHWYPQLLYRLGRLLLEEGSFHPLGMSGAGS

SEQ ID NO: 3 - DNA, 449 bp, Homo sapiens
atatccactcctgctctccctcctgcaggtgaccccagccatgaggaccatcgccatccttgctgccattctcctggtggccctgcaggcccaggctgagtcactccaggaaagagctgatgaggctacaacccagaagcagtctggggaagacaaccaggaccttgctatctcctttgcaggaaatggactctctgctcttagaacctcaggttctcaggcaagagccacctgctattgccgaaccggccgttgtgctacccgtgagtccctctccggggtgtgtgaaatcagtggccgcctctacagactctgctgtcgctgagcttcctagatagaaaccaaagcagtgcaagattcagttcaaggtcctgaaaaaagaaaaacattttactctgtgtaccttgtgtctttctaaatttctctctccaaaataaagttcaagcatt

SEQ ID NO: 4 - protein, 94 aa, Homo sapiens
MRTIAILAAILLVALQAQAESLQERADEATTQKQSGEDNQDLAISFAGNGLSALRTSGSQARATCYCRTGRCATRESLSGVCEISGRLYRLCCR

SEQ ID NO: 5 - DNA, 475 bp, Homo sapiens
acacatctgctcctgctctctctcctccagcgaccctagccatgagaaccctcaccatcctcactgctgttctcctcgtggccctccaggccaaggctgagccactccaagctgaggatgatccactgcaggcaaaagcttatgaggctgatgcccaggagcagcgtggggcaaatgaccaggactttgccgtctcctttgcagaggatgcaagctcaagtcttagagctttgggctcaacaagggctttcacttgccattgcagaaggtcctgttattcaacagaatattcctatgggacctgcactgtcatgggtattaaccacagattctgctgcctctgagggatgagaacagagagaaatatattcataatttactttatgacctagaaggaaactgtcgtgtgtcccatacattgccatcaactttgtttcctcatctcaaataaagtcctttcagcaaaaaaaaaaaa

SEQ ID NO: 6 - protein, 100 aa, Homo sapiens
MRTLTILTAVLLVALQAKAEPLQAEDDPLQAKAYEADAQEQRGANDQDFAVSFAEDASSSLRALGSTRAFTCHCRRSCYSTEYSYGTCTVMGINHRFCCL

SEQ ID NO: 7 - DNA, 20 bp, Artificial Sequence
  source: /note=“Description of Artificial Sequence Synthetic primer”
cttcagcgatgtgctcattt

SEQ ID NO: 8 - DNA, 20 bp, Artificial Sequence
  source: /note=“Description of Artificial Sequence Synthetic primer”
ctgagtctcgatgacctgga

SEQ ID NO: 9 - DNA, 20 bp, Artificial Sequence
  source: /note=“Description of Artificial Sequence Synthetic primer”
tactggaccgcgagccccac

SEQ ID NO: 10 - DNA, 19 bp, Artificial Sequence
  source: /note=“Description of Artificial Sequence Synthetic primer”
gctacccgtgagtccctct

SEQ ID NO: 11 - DNA, 20 bp, Artificial Sequence
  source: /note=“Description of Artificial Sequence Synthetic primer”
tcttgcactgctttggtttc

SEQ ID NO: 12 - DNA, 22 bp, Artificial Sequence
  source: /note=“Description of Artificial Sequence Synthetic primer”
tgtgtgaaatcagtggccgcct

SEQ ID NO: 13 - DNA, 20 bp, Artificial Sequence
  source: /note=“Description of Artificial Sequence Synthetic primer”
agagctttgggctcaacaag

SEQ ID NO: 14 - DNA, 20 bp, Artificial Sequence
  source: /note=“Description of Artificial Sequence Synthetic primer”
atgacagtgcaggtcccata

SEQ ID NO: 15 - DNA, 24 bp, Artificial Sequence
  source: /note=“Description of Artificial Sequence Synthetic primer”
cacttgccattgcagaaggtcctg

(19) United States
(12) Patent Application Publication
(10) Pub. No.: US 2009/0155788 A1
(43) Pub. Date: Jun. 18, 2009

(54) GENE EXPRESSION MARKERS FOR INFLAMMATORY BOWEL DISEASE

(76) Inventors: Alexander R. Abbas, San Carlos, CA (US); Lauri Diehl, Los Altos, CA (US); Charles Lees, Linlithgow (GB); Colin L. Noble, Edinburgh (GB); Jack Satsangi, Edinburgh (GB)

Correspondence Address: Goodwin Procter LLP;Attn: Patent Administrator, 135 Commonwealth Drive, Menlo Park, CA, 94025-1105, US

(21) Appl. No.: 12/125,724

(22) Filed: May 22, 2008

Related U.S. Application Data
(60) Provisional application No. 60/939,513, filed on May 22, 2007.
(60) Provisional application No. 60/991,203, filed on Nov. 29, 2007.

(51) Int. Cl. C12Q 1/68

(52) U.S. Cl. 435/6

(57) ABSTRACT

The present invention provides for a method of detecting the presence of inflammatory bowel disease in gastrointestinal tissues or cells of a mammal by detecting decreased expression of Indian Hedgehog (Ihh) and/or increased expression of Defensin A5 (DefA5) and/or Defensin A6 (DefA6) in the tissues or cells of the mammal relative to a control.

### CROSS-REFERENCE TO RELATED APPLICATIONS

This application is a non-provisional application filed under 37 CFR 1.53(b)(1), claiming priority under 35 USC 119(e) to U.S. Provisional Application Nos. 60/939,513, filed May 22, 2007, and 60/991,203 filed Nov. 29, 2007, the contents of which are incorporated herein by reference.

### FIELD OF THE INVENTION

The present invention relates to gene expression profiles in inflammatory bowel disease pathogenesis. This discovery finds use in the detection and diagnosis of inflammatory bowel disease, including methods for diagnosing inflammatory bowel disease in a mammal by detecting differential gene expression in tissue from the mammal.

### BACKGROUND OF THE INVENTION

Inflammatory bowel disease (IBD), a chronic inflammatory disorder of the gastrointestinal tract suffered by approximately one million patients in the United States, is made up of two major disease groups: ulcerative colitis (UC) and Crohn's Disease (CD). In both forms of IBD, intestinal microbes may initiate the disease in genetically susceptible individuals. UC is often restricted to the colon, while CD typically occurs in the ileum of the small intestine and in the colon. (Podolsky, D. K., N. Engl. J. Med. 347:417-429 (2002). Gene expression profiling of tissue from IBD patients has provided some insight into possible targets for therapy and/or diagnosis (see, for example, Dieckgraefe, B. K. et al., Physiol. Genomics 4:1-11 (2000); Lawrance I. C. et al., Hum Mol Genet. 10:445-456 (2001); Dooley T. P. et al., Inflamm. Bowel Dis. 10:1-14 (2004); and Uthoff S. M., Int J Oncol. 19:803-810 (2001)).

The vertebrate family of hedgehog genes includes at least four members or paralogs of the single Drosophila hedgehog gene (WO 95/18856 and WO 96/17924). Three of these members are Desert hedgehog (Dhh), Sonic hedgehog (Shh) and Indian hedgehog (Ihh). In mammals, hedgehog signaling occurs through the interaction of a hedgehog protein (Shh, Dhh, Ihh, collectively “Hh”) with the hedgehog receptor, patched (Ptch), and the co-receptor Smoothened (Smo), resulting in the regulation of Gli gene family transcription.

Human alpha defensins are made up of a family of polypeptides: four human neutrophil peptides (HNP) 1, 2, 3, and 4, which function in innate immunity, and two human defensins (HD) 5 and 6, which are expressed in intestinal Paneth cells and may function in innate defense of the gastrointestinal mucosa (Cunliffe, R. N., Mol. Immunol. 40:463-467 (2003)). Human alpha defensins have been shown to exhibit antimicrobial activity in vitro against some bacteria, fungi, enveloped viruses, and parasites (Ganz, T. and Weiss, J. Semin. Hematol. 34:343-354 (1997) and Ganz, T. and Lehrer, R. I. Pharmacol. Ther. 66:191-205 (1995)). Human alpha defensins 5 and 6 are stored as pro-molecules in Paneth cells which in the healthy colon are largely restricted to the terminal ileum and, on release into the mucosa, they are cleaved by trypsin to the active antimicrobial peptide (Ghosh D et al, Nat Immunol 3:583-590 (2002)). WehKamp, J. and Stange, E. F. showed that alpha defensins were expressed at reduced levels in Crohn's Disease (CD) patients (Wehkamp, J. and Stange, E. F., Ann. N.Y. Acad. Sci. 1072:321-331 (2006); Wehkamp, J., et al., PNAS USA 102:18129-18134 (2005); and Wehkamp, J. et al., Gut 53:1658-1664 (2004)). Human defensin alpha-6 has been shown to be expressed at significantly higher levels in colon tissue and serum from colon cancer patients relative to controls (Nam, M. J. et al., J. Biol. Chem. 280(9):8260-8265 (2005)). Human defensin alpha-5 has been shown to be upregulated in ulcerative colitis (Dieckgraefe, B. K. et al., Physiol. Genomics 4:1-11 (2000)).

The biological dysregulation of genes in patients experiencing inflammatory bowel disease is actively being investigated. For example, Lawrance, I. C. et al. disclosed distinctive gene expression profiles for several genes in UC and CD (Lawrance, I. C. et al., Human Mol. Genetics. 10(5):445-456 (2001)). Uthoff, S. M. S. et al. disclosed the identification of candidate genes for UC and CD using micro array analysis (Uthoff, S. M. S. et al., Int'l. J. Oncology 19:803-810 (2001). Dooley, T. P. et al. disclosed correlation of gene expression in IBD with drug treatment for the disorder (Dooley, T. P. et al., Inflamm. Bowel Dis. 10(1):1-14 (2004).

Immune related and inflammatory diseases are the manifestation or consequence of fairly complex, often multiple interconnected biological pathways which in normal physiology are critical to respond to insult or injury, initiate repair from insult or injury, and mount innate and acquired defense against foreign organisms. Disease or pathology occurs when these normal physiological pathways cause additional insult or injury either as directly related to the intensity of the response, as a consequence of abnormal regulation or excessive stimulation, as a reaction to self, or as a combination of these.

Though the genesis of these diseases often involves multistep pathways and often multiple different biological systems/pathways, intervention at critical points in one or more of these pathways can have an ameliorative or therapeutic effect. Therapeutic intervention can occur by either antagonism of a detrimental process/pathway or stimulation of a beneficial process/pathway.

Many immune related diseases are known and have been extensively studied. Such diseases include immune-mediated inflammatory diseases, non-immune-mediated inflammatory diseases, infectious diseases, immunodeficiency diseases, neoplasia, etc.

The term inflammatory bowel disorder (“IBD”) describes a group of chronic inflammatory disorders of unknown causes in which the intestine (bowel) becomes inflamed, often causing recurring cramps or diarrhea. The prevalence of IBD in the US is estimated to be about 200 per 100,000 population. Patients with IBD can be divided into two major groups, those with ulcerative colitis (“UC”) and those with Crohn's disease (“CD”). Both UC and CD are chronic relapsing diseases and are complex clinical entities that occur in genetically susceptible individuals who are exposed to as yet poorly defined environmental stimuli. (Bonen and Cho, Gastroenterology. 2003; 124:521-536; Gaya et al. Lancet. 2006; 367:1271-1284).

Although the cause of IBD remains unknown, several factors such as genetic, infectious and immunologic susceptibility have been implicated. IBD is much more common in Caucasians, especially those of Jewish descent. The chronic inflammatory nature of the condition has prompted an intense search for a possible infectious cause. Although agents have been found which stimulate acute inflammation, none has been found to cause the chronic inflammation associated with IBD. The hypothesis that IBD is an autoimmune disease is supported by the previously mentioned extraintestinal manifestation of IBD as joint arthritis, and the known positive response to IBD by treatment with therapeutic agents such as adrenal glucocorticoids, cyclosporine and azathioprine, which are known to suppress immune response. In addition, the GI tract, more than any other organ of the body, is continuously exposed to potential antigenic substances such as proteins from food, bacterial byproducts (LPS), etc.

There is sufficient overlap in the diagnostic criteria for UC and CD that it is sometimes impossible to say which a given patient has; however, the type of lesion typically seen is different, as is the localization. UC mostly appears in the colon, proximal to the rectum, and the characteristic lesion is a superficial ulcer of the mucosa; CD can appear anywhere in the bowel, with occasional involvement of stomach, esophagus and duodenum, and the lesions are usually described as extensive linear fissures.

The current therapy of IBD usually involves the administration of antinflammatory or immunosuppressive agents, such as sulfasalazine, corticosteroids, 6-mercaptopurine/azathioprine, or cyclosporine, which usually bring only partial results. If anti-inflammatory/immunosuppressive therapies fail, colectomies are the last line of defense. The typical operation for CD not involving the rectum is resection (removal of a diseased segment of bowel) and anastomosis (reconnection) without an ostomy. Sections of the small or large intestine may be removed. About 30% of CD patients will need surgery within the first year after diagnosis. In the subsequent years, the rate is about 5% per year. Unfortunately, CD is characterized by a high rate of recurrence; about 5% of patients need a second surgery each year after initial surgery.

Refining a diagnosis of inflammatory bowel disease involves evaluating the progression status of the diseases using standard classification criteria. The classification systems used in IBD include the Truelove and Witts Index (Truelove S. C. and Witts, L. J. Br Med J. 1955; 2:1041-1048), which classifies colitis as mild, moderate, or severe, as well as Lennard-Jones. (Lennard-Jones JE. Scand J Gastroenterol Suppl 1989; 170:2-6) and the simple clinical colitis activity index (SCCAI). (Walmsley et. al. Gut. 1998; 43:29-32) These systems track such variables as daily bowel movements, rectal bleeding, temperature, heart rate, hemoglobin levels, erythrocyte sedimentation rate, weight, hematocrit score, and the level of serum albumin.

In approximately 10-15% of cases, a definitive diagnosis of ulcerative colitis or Crohn's disease cannot be made and such cases are often referred to as “indeterminate colitis.” Two antibody detection tests are available that can help the diagnosis, each of which assays for antibodies in the blood. The antibodies are “perinuclear anti-neutrophil antibody” (pANCA) and “anti-Saccharomyces cervisiae antibody” (ASCA). Most patients with ulcerative colitis have the pANCA antibody but not the ASCA antibody, while most patients with Crohn's disease have the ASCA antibody but not the pANCA antibody. However, these two tests have shortcomings as some patients have neither antibody and some Crohn's disease patients may have only the pANCA antibody. For clinical practice, a reliable test that would indicate the presence and/or progression of an IBD based on molecular markers rather than the measurement of a multitude of variables would be useful for identifying and/or treating individuals with an IBD. Hypothesis free, linkage and association studies have identified genetic loci that have been associated with UC, notably the MHC region on chromosome 6, (Rioux et al. Am J Hum Genet. 2000; 66:1863-1870; Stokkers et al. Gut. 1999; 45:395-401; Van Heel et al. Hum Mol Genet. 2004; 13:763-770) the IBD2 locus on chromosome 12 (Parkes et al. Am J Hum Genet. 2000; 67:1605-1610; Satsangi et al. Nat Genet. 1996; 14:199-202) and the IBD5 locus on chromosome 5. (Giallourakis et. al. Am J. Hum Genet. 2003; 73:205-211; Palmieri et. al Aliment Pharmacol Ther. 2006; 23:497-506; Russell et. al. Gut. 2006; 55:1114-1123; Waller et. al. Gut. 2006; 55:809-814) Following a UK wide linkage scan identifying a putative loci of association for UC on chromosome 7q, further studies have implicated variants in the ABCB1 (MDR1) gene which is involved in cellular detoxification with UC. (Satsangi et. al. Nat. Genet. 1996; 14:199-202; Brant et. al. Am J Hum Genet. 2003; 73:1282-1292; Ho et. al. Gastroenterology. 2005; 128:288-296)

A complementary approach towards the identification and understanding of the complex gene-gene and gene-environment relationships that result in the chronic intestinal inflammation observed in inflammatory bowel disease (IBD) is microarray gene expression analysis. Microarrays allow a comprehensive picture of gene expression at the tissue and cellular level, thus helping understand the underlying patho-physiological processes. (Stoughton et. al. Annu Rev Biochem. 2005; 74:53-82) Microarray analysis was first applied to patients with IBD in 1997, comparing expression of 96 genes in surgical resections of patients with CD to synovial tissue of patients with rheumatoid arthritis. (Heller et. al. Proc Natl Acad Sci USA. 1997; 94:2150-2155) Further studies using microarray platforms to interrogate surgical specimens from patients with IBD identified an number of novel genes that were differentially regulated when diseased samples were compared to controls. (Dieckgraefe et. al. Physiol Genomics. 2000; 4:1-11; Lawrance et. al. Hum Mol Genet. 2001; 10:445-456)

Endoscopic pinch mucosal biopsies have allowed investigators to microarray tissue from a larger range of patients encompassing those with less severe disease. Langmann et. al. used microarray technology to analyze 22,283 genes in biopsy specimens from macroscopically non affected areas of the colon and terminal ileum. (Langmann et. al. Gastroenterology. 2004; 127:26-40) Genes which were involved in cellular detoxification and biotransformation (Pregnane X receptor and MDR1) were significantly downregulated in the colon of patients with UC, however, there was no change in the expression of these genes in the biopsies from patients with CD. Costello and colleagues (Costello et. al. PLoS Med. 2005; 2:e199) looked at the expression of 33792 sequences in endoscopic sigmoid colon biopsies obtained from healthy controls, patients with CD and UC. A number of sequences representing novel proteins were differentially regulated and in silico analysis suggested that these proteins had putative functions related to disease pathogenesis—transcription factors, signaling molecules and cell adhesion.

In a study of patients with UC, Okahara et al. (Aliment Pharmacol Ther. 2005; 21:1091-1097) observed that (migration inhibitory factor-related protein 14 (MRP14), growth-related oncogene gamma (GROγ) and serum amyloid A1 (SAA1) were upregulated where as TIMP1 and elfin were down regulated in the inflamed biopsies when compared to the non-inflamed biopsies. When observing 41 chemokines and 21 chemokine receptors, Puleston et al demonstrated that chemokines CXCLs 1-3 and 8 and CCL20 were upregulated in active colonic CD and UC. (Aliment Pharmacol Ther. 2005; 21:109-120) Overall these studies illustrate the heterogeneity of early microarray platforms and tissue collection. However, despite these problems differential expression of a number of genes was consistently observed.

Despite the above identified advances in IBD research, there is a great need for additional diagnostic and therapeutic agents capable of detecting IBD in a mammal and for effectively treating this disorder. Accordingly, the present invention provides polynucleotides and polypeptides that are differentially expressed in IBD as compared to normal tissue, and methods of using those polypeptides, and their encoding nucleic acids, for to detect or diagnose the presence of an IBD in mammalian subjects and subsequently to treat those subjects in which an IBD is detected with suitable IBD therapeutic agents. The present invention provides methods for detecting the presence of and determining the progression of inflammatory bowel disease (IBD), including ulcerative colitis (UC) and Crohn's disease (CD).

These and further embodiments of the present invention will be apparent to those of ordinary skill in the art.

The entire contents of all references cited herein are hereby incorporated by reference.

### SUMMARY OF THE INVENTION

In the broadest sense, the invention provides for a method of detecting increased expression of Human Defensin alpha 5 (DefA5 or HD 5 or HD A5) and/or increased expression of Human Defensin alpha 6 (DefA6 or HD 6 or HD A6) and/or decreased expression of Indian Hedgehog (Ihh) in intestinal tissue from a first mammal experiencing an intestinal disorder relative to a control mammal. In a more directed sense, the method is expected to be applicable to the diagnosis of disorders related to intestinal disorders associated with Ihh, DefA5 and/or DefA6 expression, which disorders include without limitation inflammatory bowel disease (IBD). In one embodiment, the method of the invention is useful to detect the presence of IBD in a mammal. In one embodiment, the IBD is ulcerative colitis (UC). In one embodiment, method of the invention is useful to detect the presence of ulcerative colitis in a mammal. In one embodiment, the IBD is Crohn's Disease (CD). In one embodiment, the method is useful to detect Crohn's Disease in a mammal. In one embodiment, the method is useful to detect responders and nonresponders of IBD therapeutic treatment. In one embodiment, the intestinal tissue is colon tissue. In one embodiment, the colon tissue is sigmoid colon. In one embodiment, the colon tissue is descending colon tissue. In one embodiment, Ihh gene expression is downregulated in an IBD or UC or CD patient relative to a control patient not experiencing IBD or UC or CD. In one embodiment, DefA5 and/or DefA6 expression is upregulated in an IBD patient relative to a control patient not experiencing IBD, UC or CD (or a control sample of normal tissue).

In one aspect, the invention concerns a method of detecting or diagnosing an inflammatory bowel disease (IBD) in a mammalian subject comprising determining, in a biological sample obtained from the subject, that an expression level of (i) one or more RNA transcripts or expression products thereof of a gene shown as SEQ ID NO: 1, SEQ ID NO:3, or SEQ ID NO:5, or (ii) one or more nucleic acids encoding a polypeptide shown as SEQ ID NO:2, SEQ ID NO:4, or SEQ ID NO:6 is different relative to an expression level in a control, wherein the difference in expression indicates the subject is more likely to have an IBD.

In one aspect, the invention concerns a method of detecting or diagnosing an inflammatory bowel disease (IBD) in a mammalian subject comprising determining, in a biological sample obtained from the subject, that an expression level of (i) an RNA transcript or expression product thereof of a gene shown as SEQ ID NO: 1; or (ii) a nucleic acid encoding a polypeptide shown as SEQ ID NO:2 is lower relative to an expression level in a control, wherein the lower expression indicates the subject is more likely to have an IBD.

In one aspect, the invention concerns a method of detecting or diagnosing an inflammatory bowel disease (IBD) in a mammalian subject comprising determining, in a biological sample obtained from the subject, that an expression level of (i) one or more RNA transcripts or expression products thereof of a gene shown as SEQ ID NO:3 or SEQ ID NO:5, or (ii) one or more nucleic acids encoding a polypeptide shown as SEQ ID NO:4 or SEQ ID NO:6 is higher relative to an expression level in a control, wherein the higher expression indicates the subject is more likely to have an IBD.

In one aspect, the invention concerns a method of detecting or diagnosing an inflammatory bowel disease (IBD) in a mammalian subject comprising (a) determining, in a biological sample obtained from the subject, that an expression level of (i) an RNA transcript or expression product thereof of a gene shown as SEQ ID NO: 1; or (ii) a nucleic acid encoding a polypeptide shown as SEQ ID NO:2 is lower relative to an expression level in a control, wherein the lower expression indicates the subject is more likely to have an IBD; and (b) determining, in a biological sample obtained from the subject, that an expression level of (i) one or more RNA transcripts or expression products thereof of a gene shown as SEQ ID NO:3 or SEQ ID NO:5, or (ii) one or more nucleic acids encoding a polypeptide shown as SEQ ID NO:4 or SEQ ID NO:6 is higher relative to an expression level in a control, wherein the higher expression indicates the subject is more likely to have an IBD.

In one aspect, the methods are directed to diagnosing or detecting a flare-up of an IBD in mammalian subject that was previously diagnosed with an IBD and is currently in remission. The subject may have completed treatment for the IBD or is currently undergoing treatment for the IBD. In one aspect, the invention concerns a method of detecting or diagnosing an inflammatory bowel disease (IBD) in a mammalian subject comprising determining, in a biological sample obtained from the subject, that an expression level of (i) one or more RNA transcripts or expression products thereof of a gene shown as SEQ ID NO:1, SEQ ID NO:3, or SEQ ID NO:5, or (ii) one or more nucleic acids encoding a polypeptide shown as SEQ ID NO:2, SEQ ID NO:4, or SEQ ID NO:6 is different relative to an expression level in a control, wherein the difference in expression indicates the subject is more likely to have an IBD flareup. Alternatively, the test sample may be compared to a prior test sample of the mammalian subject, if available, obtained before, after, or at the time of the initial IBD diagnosis.

In all aspects, the mammalian subject preferably is a human patient, such as a human patient diagnosed with or at risk of developing an IBD. The subject may also be an IBD patient who has received prior treatment for an IBD but is at risk of a recurrence of the IBD.

For all aspects of the method of the invention, determining an expression level of one or more genes described herein (or one or more nucleic acids encoding polypeptide(s) expressed by one or more of such genes) may be obtained, for example, by a method of gene expression profiling. The method of gene expression profiling may be, for example, a PCR-based method.

In various embodiments, the diagnosis includes quantification of an expression level of (i) one or more RNA transcripts or expression products thereof of a gene shown as SEQ ID NO:1, SEQ ID NO:3, or SEQ ID NO:5 or (ii) one or more nucleic acids encoding a polypeptide shown as SEQ ID NO:2, SEQ ID NO:4, or SEQ ID NO:6, such as by immunohistochemistry (IHC) and/or fluorescence in situ hybridization (FISH).

For all aspects of the invention, the expression levels of the genes may be normalized relative to the expression levels of one or more reference genes, or their expression products.

In another aspect, the methods of present invention also contemplate the use of a “panel” of such genes (i.e. IBD markers as disclosed herein) based on the evidence of their level of expression. In some embodiments, the panel of IBD markers will include at least 1 IBD marker, at least two IBD markers, or at least three IBD markers. The panel may include an IBD marker that is overexpressed in IBD relative to a control, an IBD marker that is underexpressed in IBD relative to a control, or IBD markers that are both overexpressed and underexpressed in IBD relative to a control. Such panels may be used to screen a mammalian subject for the differential expression of one or more IBD markers in order to make a determination on whether an IBD is present in the subject.

In one embodiment, the IBD markers that make up the panel are selected from (i) one or more genes shown as SEQ ID NO:1, SEQ ID NO:3, or SEQ ID NO:5, or (ii) one or more nucleic acids encoding a polypeptide shown as SEQ ID NO:2, SEQ ID NO:4, or SEQ ID NO:6. In a preferred embodiment, the methods of diagnosing or detecting the presence of an IBD in a mammalian subject comprise determining a differential expression level of (i) one or more RNA transcripts or expression products thereof; or (ii) one or more nucleic acids encoding a polypeptide from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

For all aspects of the invention, the method may further comprise the step of creating a report summarizing said prediction.

For all aspects, the IBD diagnosed or detected according to the methods of the present invention is Crohn's disease (CD), ulcerative colitis (UC), or both CD and UC.

For all aspects of the invention, the test sample obtained from a mammalian subject may be derived from a colonic tissue biopsy. In a preferred embodiment, the biopsy is a tissue selected from the group consisting of terminal ileum, the ascending colon, the descending colon, and the sigmoid colon. In other preferred embodiments, the biopsy is from an inflamed colonic area or from a non-inflamed colonic area. The inflamed colonic area may be acutely inflamed or chronically inflamed.

For all aspects, determination of expression levels may occur at more than one time. For all aspects of the invention, the determination of expression levels may occur before the patient is subjected to any therapy before and/or after any surgery. In some embodiments, the determining step is indicative of a recurrence of an IBD in the mammalian subject following surgery or indicative of a flare-up of said IBD in said mammalian subject. In a preferred embodiment, the IBD is Crohn's disease.

In another aspect, the present invention concerns methods of treating a mammalian subject in which the presence of an IBD has been detected by the methods described herein. For example, following a determination that a test sample obtained from the mammalian subject exhibits differential expression relative to a control of one or more of the RNA transcripts or the corresponding gene products of an IBD marker described herein, the mammalian subject may be administered an IBD therapeutic agent.

In one embodiment, the methods of treating an IBD in a mammalian subject in need thereof, comprise (a) determining a differential level of expression of (i) one or more RNA transcripts or expression products thereof of a gene shown as SEQ ID NO:1, SEQ ID NO:3, or SEQ ID NO:5, or (ii) one or more nucleic acids encoding a polypeptide shown as SEQ ID NO:2, SEQ ID NO:4, or SEQ ID NO:6, in a test sample obtained from said subject relative to an level of expression in a control, wherein said differential level of expression is indicative of the presence of an IBD in the subject from which the test sample was obtained; and (b) administering to said subject an effective amount of an IBD therapeutic agent.

In a preferred embodiment, the methods of treating an IBD comprise (a) determining that an expression level of (i) an RNA transcript or expression product thereof of a gene shown as SEQ ID NO:1, or (ii) a nucleic acid encoding a polypeptide shown as SEQ ID NO:6 in a test sample obtained from the subject is lower relative to a level of expression in a control, wherein the lower level of expression is indicative of the presence of an IBD in the subject from which the test sample was obtained; and (b) administering to said subject an effective amount of an IBD therapeutic agent. In another preferred embodiment, the methods of treating an IBD comprise (a) determining that an expression level of (i) one or more RNA transcripts or expression products thereof of a gene shown as SEQ ID NO:3 or SEQ ID NO:5, or (ii) one or more nucleic acids encoding a polypeptide shown as SEQ ID NO:4 or SEQ ID NO:6 in a test sample obtained from the subject is higher relative to a level of expression in a control, wherein the higher level of expression is indicative of the presence of an IBD in the subject from which the test sample was obtained; and (b) administering to said subject an effective amount of an IBD therapeutic agent.

In one other preferred embodiment, the methods of treating an IBD comprise (a) determining that an expression level of (i) an RNA transcript or expression product thereof of a gene shown as SEQ ID NO:1, or (ii) a nucleic acid encoding a polypeptide shown as SEQ ID NO:2 in a test sample obtained from the subject is lower relative to an expression level of a control, wherein the lower level of expression is indicative of the presence of an IBD in the subject from which the test sample was obtained; (b) determining that an expression level of (i) one or more RNA transcripts or expression products thereof of a gene shown as SEQ ID NO:3 or SEQ ID NO:5, or (ii) one or more nucleic acids encoding a polypeptide shown as SEQ ID NO:4 or SEQ ID NO:6 in a test sample obtained from the subject is higher relative to a level of expression in a control, wherein the higher level of expression is indicative of the presence of an IBD in the subject from which the test sample was obtained; and (c) administering to said subject an effective amount of an IBD therapeutic agent.

In all embodiments, the IBD therapeutic agent is one or more of an aminosalicylate, a corticosteroid, and an immunosuppressive agent.

In one aspect, the panel of IBD markers discussed above is useful in methods of treating an IBD in a mammalian subject. In one embodiment, the mammalian subject is screened against the panel of markers and if the presence of an IBD is determined, IBD therapeutic agent(s) may be administered as discussed herein.

In a different aspect the invention concerns a kit comprising one or more of (1) extraction buffer/reagents and protocol; (2) reverse transcription buffer/reagents and protocol; and (3) qPCR buffer/reagents and protocol suitable for performing the methods of this invention. The kit may comprise data retrieval and analysis software.

In one embodiment, the method of the invention comprises obtaining a tissue sample from a test mammal suspected of experiencing an intestinal disorder, contacting the tissue with a detectable agent that interacts with Ihh, DefA5 or DefA6 protein (shown as SEQ ID NOS: 1, 3, and 5, respectively) or with nucleic acid encoding Ihh, DefA5 or DefA6 (shown as SEQ ID NOS: 2, 4, and 6, respectively), and determining the level of Ihh, DefA5 or DefA6 expression relative to a control tissue. In one embodiment, increased DefA5 and/or DefA6 expression relative to control and/or decreased expression of Ihh relative to control is indicative of IBD in the test mammal. In one embodiment, increased DefA5 and/or DefA6 expression relative to control and/or decreased expression of Ihh relative to control is indicative of UC in the test mammal. In one embodiment the tissue or cells from the test mammal are from the colon. In one embodiment, the tissue or cells from the test mammal are at least from the ascending colon. In one embodiment, the tissue or cells from the test mammal are at least from the sigmoid colon. In one embodiment, the tissue or cells from the test mammal are at least from the descending colon.

In one aspect, the invention concerns an article of manufacture comprising a container and a composition of matter contained within the container, wherein the composition of matter comprises a nucleic acid encoding Ihh, DefA5 and/or DefA6 or their complements, or a portion thereof comprising at least 20 contiguous nucleotides and useful as a hybridization probe, and/or an anti-Ihh antibody, an anti-DefA5 and/or an anti-DefA6 antibody, or binding fragment thereof, wherein the nucleic acids and/or antibodies are detectable. In one embodiment, the composition of matter further comprises agents for detecting nucleic acid binding, such as without limitation Ihh-, DefA5- and/or DefA6-encoding nucleic acids or their complements, or antibodies to Ihh, DefA5 and/or DefA6 polypeptides in a tissue sample of a test mammal suspected of experiencing an intestinal disorder. In one embodiment, the detecting agent nucleic acid or antibody of the composition is detectably labeled or may be labeled after binding. In one embodiment, the antibody of the composition is detectable by a second antibody, which second antibody is detectable or detectably labeled. The article may further optionally comprise a label affixed to the container, or a package insert included with the container, that refers to the use the Ihh, DefA5 and/or DefA6 nucleic acid or its complement and/or the anti-Ihh antibody, anti-DefA5 antibody or anti-DefA6 antibody or binding fragment thereof in the diagnostic detection of an IBD, including without limitation, UC.

In yet a further embodiment, the present invention concerns a method of diagnosing the presence of an intestinal disorder in a mammal, comprising detecting the level of expression of a gene encoding an Ihh, DefA5, or a DefA6 polypeptide (a) in a test sample of tissue or cells obtained from said mammal, and (b) in a control sample of normal cells from a mammal not experiencing an intestinal disorder of the same tissue origin or type, wherein a lower level of expression of the Ihh polypeptide and/or an increased level of expression of DefA5 and/or DefA6 polypeptide in the test sample, as compared to the control sample, is indicative of the presence of an intestinal disorder in the mammal from which the test sample was obtained. In an embodiment, the intestinal disorder in IBD. In an embodiment, the IBD is UC.

In yet a further embodiment, the present invention concerns a method of diagnosing the presence of an intestinal disorder in a mammal, comprising (a) contacting a test sample comprising tissue or cells obtained from the mammal with an antibody, oligopeptide or small organic molecule that binds to an Ihh, DefA5 and/or a DefA6 nucleic acid (or its complement) or an Ihh, DefA5 and/or a DefA6 polypeptide and (b) detecting the formation of a complex between the antibody, oligopeptide or small organic molecule and the Ihh, and/or a DefA6 nucleic acid (or its complement) or polypeptide in the test sample, wherein the formation of less Ihh complex in the sample relative to a control sample is indicative of the presence of an intestinal disorder in the mammal and/or wherein an increased formation of DefA5 and/or DefA6 complex in the sample relative to a control sample is indicative of the presence of an intestinal disorder in the mammal. In one embodiment, the intestinal disorder is IBD. In one embodiment, the disorder is UC. In one embodiment, the disorder is CD. Optionally, the antibody, Ihh, DefA5 and/or DefA6 binding oligopeptide, or binding organic molecule employed is detectable, detectably labeled, attached to a solid support, or the like, and/or the test sample of tissue or cells is obtained from an individual suspected of experiencing an intestinal disorder, wherein the disorder is IBD, such as without limitation, UC or CD.

In yet a further embodiment, the present invention concerns the use of (a) an Ihh polypeptide, (b) a nucleic acid encoding an Ihh polypeptide or a vector or host cell comprising the nucleic acid, (c) an anti-Ihh polypeptide antibody, (d) an Ihh-binding oligopeptide, or (e) an Ihh-binding small organic molecule in the preparation of a medicament useful for the diagnostic detection of an intestinal disorder, including without limitation, IBD, such as UC or CD.

In one embodiment, the present invention concerns the use of (a) a DefA5 polypeptide, (b) a nucleic acid encoding a DefA5 polypeptide or a vector or host cell comprising the nucleic acid, (c) an anti-DefA5 polypeptide antibody, (d) a DefA5-binding oligopeptide, or (e) a DefA5-binding small organic molecule in the preparation of a medicament useful for the diagnostic detection of an intestinal disorder, including without limitation, IBD, such as CD or UC.

In one embodiment, the present invention concerns the use of (a) a DefA6 polypeptide, (b) a nucleic acid encoding a DefA6 polypeptide or a vector or host cell comprising the nucleic acid, (c) an anti-DefA6 polypeptide antibody, (d) a DefA6-binding oligopeptide, or (e) a DefA6-binding small organic molecule in the preparation of a medicament useful for the diagnostic detection of an intestinal disorder, including without limitation, IBD, such as CD or UC.

In one aspect, decreased Ihh expression may be determined by the underexpression of a hedgehog gene or the presence of a mutated or dysfunctional hedgehog gene (e.g., ptch-1, ptch-2, Smo, Fu, Su(Fu), etc.).

In one aspect, increased DefA5 or DefA6 expression may be determined by the increased expression of DefA5 or DefA6 or the presence of a mutated or dysfunctional DefA5 or DefA6 gene. In one embodiment increased expression of DefA5 and/or DefA6 is determined at a plurality of locations along the gastrointestinal tract, wherein an increase in DefA5 and/or DefA6 in ascending, descending and sigmoid colon of inflamed test samples relative to control samples indicates the presence of UC in the patient from whom the test sample was obtained.

In one aspect, the invention comprises a method of detecting a therapeutic drug response in a mammal treated with an IBD therapeutic agent, wherein the method comprises determining Ihh, DefA5 and/or DefA6 expression in gastrointestinal tissue of a test mammal relative to a control and determining that the Ihh, DefA5 and/or DefA6 expression levels are not significantly different from normal control expression levels or are within a range of normal expression levels for Ihh, DefA5 and/or DefA6 in a population of mammals. In one embodiment, a therapeutic response is determined when the levels of expression of Ihh, DefA5 and/or DefA6 in gastrointestinal, colonic, or sigmoid colonic tissues or cells of the mammal treated with a therapeutic agent are different (expression is more similar to normal control, i.e., Ihh levels are higher, and/or DefA5 and/or DefA6 levels are lower) than Ihh, DefA5 and/or DefA6 expression levels, respectively, were in the mammal prior to treatment.

Yet further embodiments of the present invention will be evident to the skilled artisan upon a reading of the present specification.

In one embodiment, the present invention contemplates the following set of exemplary claims.

1. A method of diagnosing the presence of inflammatory bowel disease (IBD) in a mammal, comprising detecting the level of expression of at least one gene (a) in a test sample of tissue or cells obtained from said mammal, and (b) in a control sample of non-IBD tissue or cells of the same tissue origin or type; wherein an altered level of expression of the gene in the test sample, as compared to the control sample, is indicative of the presence of IBD in the mammal from which the test sample was obtained, wherein the gene encodes an Indian hedgehog (Ihh) polypeptide (SEQ ID NO:2), a Defensin alpha 5 (DefA5) polypeptide (SEQ ID NO:4), or a Defensin alpha 6 (DefA6) polypeptide (SEQ ID NO:6).

2. The method of claim 1, wherein the tissue or cells of the test sample are from the gastrointestinal tract of the mammal, and the IBD is ulcerative colitis.

3. The method of claim 2, wherein the tissue or cells of the test sample are from the colon of the mammal.

4. The method of claim 3, wherein the tissue or cells of the test sample are from a region of the colon selected from the ascending colon, the sigmoid colon, and the descending colon of the mammal.

5. The method of claim 2, wherein the altered level of expression of the gene is in the colon of the mammal.

6. The method of claim 5, wherein the altered level of expression of the gene is in a region of the colon selected from the ascending colon, the sigmoid colon, and the descending colon.

7. The method of claim 6, wherein the altered level of expression of the gene is in any two regions of the colon selected from the ascending colon, the sigmoid colon, and the descending colon of the mammal.

8. The method of claim 7, wherein the altered level of expression of the gene is in the ascending colon, the sigmoid colon, and the descending colon of the mammal.

9. The method of claim 1, wherein the tissue or cells of the test sample are inflamed.

10. The method of claim 1, wherein the tissue or cells of the test sample are not inflamed.

11. The method of claim 2, wherein the gene encodes Ihh polypeptide and the altered level of expression is a reduction in expression relative to the control sample, wherein the reduction is at least 1.5 fold.

12. The method of claim 2, wherein the gene encodes DefA5 and the altered level of expression is an increase in expression relative to the control sample, wherein the increase is at least 1.5 fold.

13. The method of claim 2, wherein the gene encodes DefA6 and the altered level of expression is an increase in expression relative to the control sample, wherein the increase is at least 1.5 fold.

14. The method of claim 1, comprising:

(a) contacting the test sample with a detectable agent that specifically binds a polynucleotide of the gene or fragment thereof, (b) contacting the control sample with the detectable agent; and (c) detecting the formation of a complex between the agent and the polynucleotide of the test sample and the control sample, wherein the formation of a different amount of complex in the test sample relative to the control sample is indicative of the presence of IBD in the mammal, wherein the difference is at least 1.5 fold.

15. The method of claim 14, wherein there is a lower amount of complex in the test sample relative to the control sample, and wherein the gene is Ihh.

16. The method of claim 14, wherein there is a greater amount of complex in the test sample relative to the control sample, and wherein the gene is DefA5 or DefA6.

17. The method of claim 14, wherein the polynucleotide comprises the coding sequence of the nucleic acid sequence of SEQ ID NOs: 1, 3 or 5 or a fragment thereof comprising at least 15 contiguous nucleotides of SEQ ID NO: 1, 3 or 5.

18. The method of claim 14, wherein the agent is a second polynucleotide that hybridizes to a polynucleotide having the sequence of the coding sequence of any one of SEQ ID NOs: 1, 3 or 5, or its complement or a fragment thereof.

19. The method of claim 1, wherein the second polynucleotide comprises a detectable label or attached to a solid support.

20. The method of claim 18, wherein the detectable label is directly detectable.

21. The method of claim 18, wherein the detectable label is indirectly detectable.

22. The method of claim 18, wherein the detectable label is a fluorescent label or a radioisotope.

23. The method of claim 14, wherein the method is in situ hybridization assay.

24. The method of claim 14, wherein the method is real time polymerase chain reaction (RT-PCR) assay.

25. The method of claim 1, comprising:

(a) contacting the test sample with a detectable agent that specifically binds a polypeptide or fragment thereof; (b) contacting the control sample with the detectable agent; and (c) detecting the formation of a complex between the agent and the polypeptide of the test sample and the control sample, wherein the formation of a different amount of complex in the test sample relative to the control sample is indicative of the presence of IBD in the mammal, wherein the polypeptide, or fragment thereof, is encoded by the Ihh, DefA5 or DefA6 gene, or fragment thereof.

26. The method of claim 25, wherein the amount of complex in the test sample is at least 1.5 fold less than the amount of complex in the control sample, and wherein the polypeptide is Ihh comprising SEQ ID NO:2 or a fragment thereof comprising at least 10 contiguous amino acids of SEQ ID NO:2.

27. The method of claim 25, wherein the amount of complex in the test sample is at least 1.5 fold greater than the amount of complex in the control sample, and wherein the polypeptide is DefA5 comprising SEQ ID NO:4 or a fragment thereof comprising at least 10 contiguous amino acids of SEQ ID NO:4, or DefA6 comprising SEQ ID NO:6 or a fragment thereof comprising at least 10 contiguous amino acids of SEQ ID NO:6.

28. The method of claim 26, wherein the agent comprises an Ihh binding portion of an Ihh receptor.

29. The method of claim 28, wherein the Ihh receptor is Patched (PTCH).

30. The method of claim 27, wherein the agent comprises a DefA5 or DefA6 binding portion of a DefA5 or DefA6 receptor.

31. The method of claim 1, claim 14, or claim 25, wherein the tissues or cells of the mammal have been contacted with a therapeutic agent, wherein the detecting is a second or subsequent detecting of Ihh, DefA5 and/or DefA6 expression in the mammal, and wherein the level of Ihh, DefA5 and/or DefA6 expression is indicative of the presence or absence of a response to the therapeutic agent in the tissue or cells of the mammal.

32. The method of claim 1, claim 14, or claim 25, wherein the IBD is ulcerative colitis (UC).

33. The method of claim 1, claim 14, or claim 25, wherein the IBD is Crohn's Disease (CD).

### BRIEF DESCRIPTION OF THE DRAWINGS

FIGS. 1A and IB depict a nucleic acid sequence (SEQ ID NO: 1) encoding human Ihh polypeptide and the amino acid sequence of human Ihh polypeptide (SEQ ID NO:2), respectively.

FIGS. 2A and 2B depict a nucleic acid sequence (SEQ ID NO:3) encoding human DefA5 polypeptide and the amino acid sequence of human DefA5 polypeptide (SEQ ID NO:4), respectively.

FIGS. 3A and 3B depict a nucleic acid sequence (SEQ ID NO:5) encoding human DefA6 polypeptide and the amino acid sequence of human DefA6 polypeptide (SEQ ID NO:6), respectively.

FIG. 4 is a graph showing the results of quantitative analysis of mRNA levels of Indian hedgehog (Ihh) in control versus ulcerative colitis colon samples. Intestinal location is identified as sigmoid colon (SC). Disease specimens were sub-categorised into non-inflamed (N-I) and inflamed (I) tissues. Individual data points were plotted with horizontal lines representing the means for each dataset.

FIG. 5 is a plot of real time PCR expression data of DefA5 in healthy control sigmoid colon samples exhibiting normal histology, non-inflamed ulcerative colitis sigmoid colon samples, and ulcerative colitis samples of sigmoid colon exhibiting acute or chronic inflammatory cell infiltrate. Standard error for each dataset is indicated. p values between data sets are indicated.

FIG. 6 is a plot of real time PCR expression data of DefA6 in healthy control sigmoid colon samples exhibiting normal histology, non-inflamed ulcerative colitis sigmoid colon samples, and ulcerative colitis samples of sigmoid colon exhibiting acute or chronic inflammatory cell infiltrate. Standard error for each dataset is indicated. p values between data sets are indicated.

FIGS. 7A-E show histology photomicrographs of DefA6 staining in the small intestine and sigmoid colon of an ulcerative colitis (UC) patient. FIG. 7A, small intestine; FIG. 7B, small intestine (isotype control); FIG. 7C, sigmoid colon (control patient); FIG. 7D, Sigmoid colon (UC patient); FIG. 7E, sigmoid colon (UC patient). Tissues were stained for the presence of DefA6. Arrows indicate positive staining in crypt epithelial cells.

FIG. 8 shows in situ hybridization of defensin alpha 5 in the terminal ileum and colon of patients with ulcerative colitis and controls.

FIG. 9 shows immunohistochemistry of defensin alpha 6 in the terminal ileum and colon of patients with ulcerative colitis and controls.

FIG. 10 shows the expression of defensins alpha 5 and 6 in ulcerative colitis patients and controls.

### DETAILED DESCRIPTION OF THE INVENTION

### Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “inflammatory bowel disease” or “IBD” is used collectively and/or interchangeably herein to refer to diseases of the bowel that cause inflammation and/or ulceration and includes without limitation Crohn's disease and ulcerative colitis. Although the two diseases are generally considered as two different entities, their common characteristics, such as patchy necrosis of the surface epithelium, focal accumulations of leukocytes adjacent to glandular crypts, and an increased number of intraepithelial lymphocytes (IEL) and certain macrophage subsets, justify their treatment as a single disease group.

The term “Crohn's disease” or “CD” is used herein to refer to a condition involving chronic inflammation of the gastrointestinal tract. Crohn's-related inflammation usually affects the intestines, but may occur anywhere from the mouth to the anus. CD differs from UC in that the inflammation extends through all layers of the intestinal wall and involves mesentery as well as lymph nodes. The disease is often discontinuous, i.e., severely diseased segments of bowel are separated from apparently disease-free areas. In CD, the bowel wall also thickens which can lead to obstructions, and the development of fistulas and fissures are not uncommon. As used herein, CD may be one or more of several types of CD, including without limitation, ileocolitis (affects the ileum and the large intestine); ileitis (affects the ileum); gastroduodenal CD (inflammation in the stomach and the duodenum); jejunoileitis (spotty patches of inflammation in the jejunum); and Crohn's (granulomatous) colitis (only affects the large intestine). Crohn's disease, unlike ulcerative colitis, can affect any part of the bowel. The most prominent feature Crohn's disease is the granular, reddish-purple edmatous thickening of the bowel wall. With the development of inflammation, these granulomas often lose their circumscribed borders and integrate with the surrounding tissue. Diarrhea and obstruction of the bowel are the predominant clinical features. As with ulcerative colitis, the course of Crohn's disease may be continuous or relapsing, mild or severe, but unlike ulcerative colitis, Crohn's disease is not curable by resection of the involved segment of bowel. Most patients with Crohn's disease require surgery at some point, but subsequent relapse is common and continuous medical treatment is usual. Crohn's disease may involve any part of the alimentary tract from the mouth to the anus, although typically it appears in the ileocolic, small-intestinal or colonic-anorectal regions. Histopathologically, the disease manifests by discontinuous granulomatomas, crypt abscesses, fissures and aphthous ulcers. The inflammatory infiltrate is mixed, consisting of lymphocytes (both T and B cells), plasma cells, macrophages, and neutrophils. There is a disproportionate increase in IgM- and IgG-secreting plasma cells, macrophages and neutrophils.

The term “ulcerative colitis” or “UC” is used herein to refer to a condition involving inflammation of the large intestine and rectum. UC afflicts the large intestine. The course of the disease may be continuous or relapsing, mild or severe. The earliest lesion is an inflammatory infiltration with abscess formation at the base of the crypts of Lieberkühn. Coalescence of these distended and ruptured crypts tends to separate the overlying mucosa from its blood supply, leading to ulceration. Symptoms of the disease include cramping, lower abdominal pain, rectal bleeding, and frequent, loose discharges consisting mainly of blood, pus and mucus with scanty fecal particles. A total colectomy may be required for acute, severe or chronic, unremitting ulcerative colitis. The clinical features of UC are highly variable, and the onset may be insidious or abrupt, and may include diarrhea, tenesmus and relapsing rectal bleeding. With fulminant involvement of the entire colon, toxic megacolon, a life-threatening emergency, may occur. Extraintestinal manifestations include arthritis, pyoderma gangrenoum, uveitis, and erythema nodosum. In patients with UC, there is an inflammatory reaction primarily involving the colonic mucosa. The inflammation is typically uniform and continuous with no intervening areas of normal mucosa. Surface mucosal cells as well as crypt epithelium and submucosa are involved in an inflammatory reaction with neutrophil infiltration. Ultimately, this reaction typically progresses to epithelial damage and loss of epithelial cells resulting in multiple ulcerations, fibrosis, dysplasia and longitudinal retraction of the colon.

The term “inactive” IBD is used herein to mean an IBD that was previously diagnosed in an individual but is currently in remission. This is in contrast to an “active” IBD in which an individual has been diagnosed with and IBD but has not undergone treatment. In addition, the active IBD may be a recurrence of a previously diagnosed and treated IBD that had gone into remission (i.e. become an inactive IBD). Such recurrences may also be referred to herein as “flare-ups” of an IBD. Mammalian subjects having an active autoimmune disease, such as an IBD, may be subject to a flare-up, which is a period of heightened disease activity or a return of corresponding symptoms. Flare-ups may occur in response to severe infection, allergic reactions, physical stress, emotional trauma, surgery, or environmental factors.

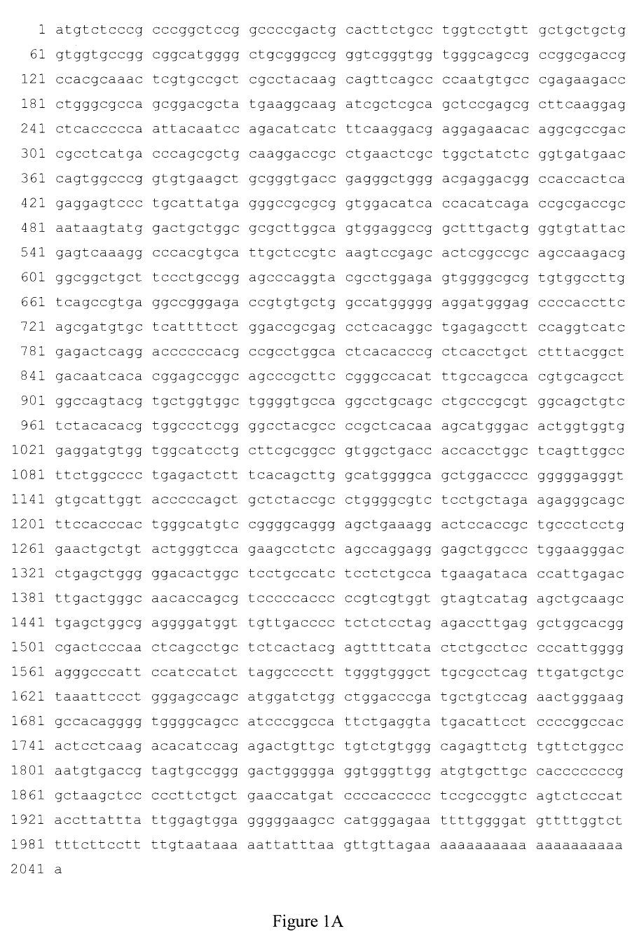

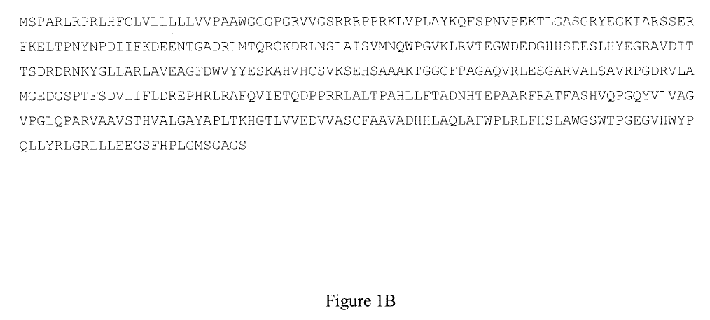

As used herein, “Indian Hedgehog,” “Indian Hedgehog homolog (Drosophila),” “Ihh” and the like are used interchangeably to refer to the Indian Hedgehog gene. In one embodiment the Ihh gene is human IHH. In one embodiment, Ihh is encoded by nucleic acid associated with GenBank Ref.Seq. number NM_{—}002181 (shown in FIG. 1A, SEQ ID NO:1). In one embodiment, Ihh is a polypeptide comprising the amino acid sequence, or fragments thereof, associated with GenBank Ref.Seq. number NM_{—}002181 (shown in FIG. 1B, SEQ ID NO:2). In one embodiment, the Ihh polynucleotide comprises at least 15, at least 25, at least, at least 50, at least 100, at least 250, at least 500, at least 750, at least 1000, at least 1250, at least 1500, at least 1750, at least 2000, or at least 2040 contiguous nucleotides of SEQ ID NO:1, or the Ihh polynucleotide comprises SEQ ID NO:1). In one embodiment, a polynucleotide that binds an Ihh polynucleotide (SEQ ID NO:1), or fragment thereof, has at least 75%, at least 80%, at least 85%, at least 90%, at least 95%, at least 97%, at least 99% or 100% sequence identity with the Ihh polypeptide or fragment thereof. In one embodiment, the Ihh polypeptide comprises at least 10, at least 25, at least 50, at least 75, at least 100, at least 125, at least 150, at least 175, at least 200, at least 225, at least 250, at least 275, at least 300, or at least 325, at least contiguous amino acids of SEQ ID NO:2, or the IHH polypeptide comprises SEQ ID NO:2).

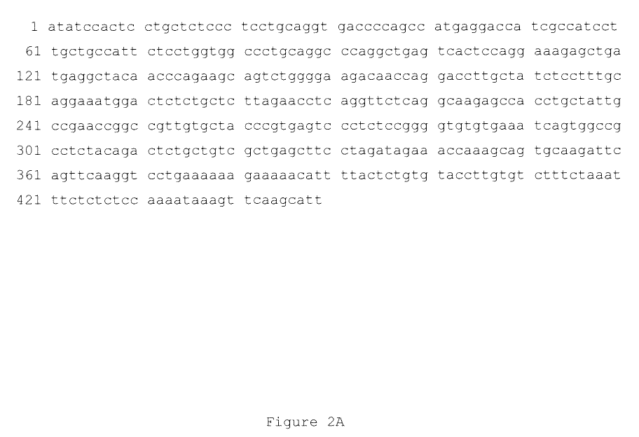

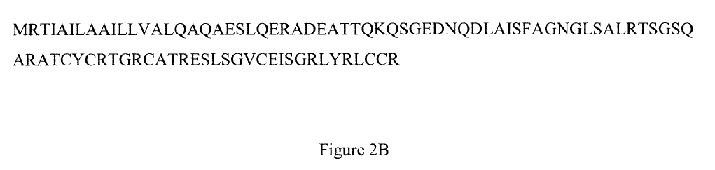

As used herein, “Defensin alpha 5,” “Human Defensin alpha 5,” “DefA5”, “HD-5” and the like are used interchangeably to refer to the human DefA5 gene. In one embodiment the DefA5 gene is human DefA5. In one embodiment, DefA5 is encoded by nucleic acid associated with GenBank Ref.Seq. number NM_{—}021010 (shown in FIG. 2A, SEQ ID NO:3). In one embodiment, DefA5 is a polypeptide comprising the amino acid sequence, or fragments thereof, associated with GenBank Ref.Seq. number NM_{—}021010 (shown in FIG. 2B, SEQ ID NO:4).

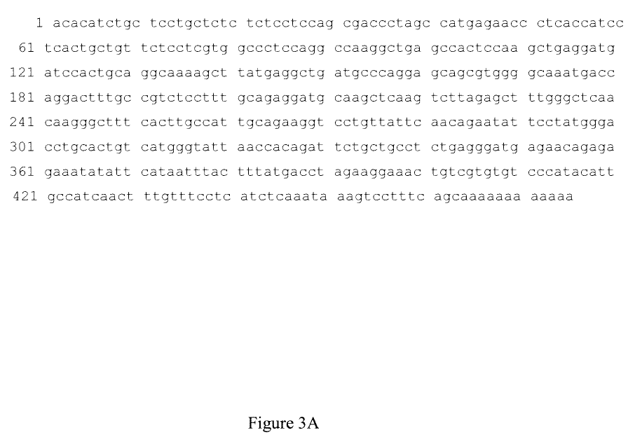

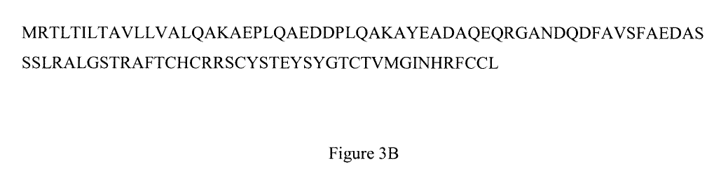

As used herein, “Defensin alpha 6,” “Human Defensin alpha 6,” “DefA6”, “HD-6” and the like are used interchangeably to refer to the human DefA6 gene. In one embodiment the DefA6 gene is human DefA6. In one embodiment, DefA6 is encoded by nucleic acid associated with GenBank Ref.Seq. number NM_{—}001926 (shown in FIG. 3A, SEQ ID NO:5). In one embodiment, DefA6 is a polypeptide comprising the amino acid sequence, or fragments thereof, associated with GenBank Ref. Seq. number NM_{—}001926 (shown in FIG. 3B, SEQ ID NO:6).

A “native sequence Ihh polypeptide” comprises a polypeptide having the same amino acid sequence as the corresponding Ihh polypeptide derived from nature. Such native sequence Ihh polypeptides can be isolated from nature or can be produced by recombinant or synthetic means. The term “native sequence Ihh polypeptide” specifically encompasses naturally-occurring truncated or secreted forms of the specific Ihh polypeptide (e.g., an extracellular domain sequence), naturally-occurring variant forms (e.g., alternatively spliced forms) and naturally-occurring allelic variants of the polypeptide. In one specific aspect, the native sequence Ihh polypeptides disclosed herein are mature or full-length native sequence polypeptides corresponding to the sequences in FIGS. 1A and 1B.

A “native sequence DefA5 polypeptide” comprises a polypeptide having the same amino acid sequence as the corresponding DefA5 polypeptide derived from nature. Such native sequence DefA5 polypeptides can be isolated from nature or can be produced by recombinant or synthetic means. The term “native sequence Ihh polypeptide” specifically encompasses naturally-occurring truncated or secreted forms of the specific DefA5 polypeptide (e.g., an extracellular domain sequence), naturally-occurring variant forms (e.g., alternatively spliced forms) and naturally-occurring allelic variants of the polypeptide. In one specific aspect, the native sequence DefA5 polypeptides disclosed herein are mature or full-length native sequence polypeptides corresponding to the sequences in FIGS. 2A and 2B.

A “native sequence DefA6 polypeptide” comprises a polypeptide having the same amino acid sequence as the corresponding DefA6 polypeptide derived from nature. Such native sequence DefA6 polypeptides can be isolated from nature or can be produced by recombinant or synthetic means. The term “native sequence Ihh polypeptide” specifically encompasses naturally-occurring truncated or secreted forms of the specific DefA6 polypeptide (e.g., an extracellular domain sequence), naturally-occurring variant forms (e.g., alternatively spliced forms) and naturally-occurring allelic variants of the polypeptide. In one specific aspect, the native sequence DefA6 polypeptides disclosed herein are mature or full-length native sequence polypeptides corresponding to the sequences in FIGS. 3A and 3B.

As used herein, a “Ihh polypeptide variant,” a “DefA5 polypeptide variant,” or a “DefA6 polypeptide variant” means an Ihh, DefA5 or DefA6 polypeptide, respectively, preferably active forms thereof, as defined herein, having at least about 80% amino acid sequence identity with a full-length native sequence Ihh, DefA5 or DefA6 polypeptide sequence, respectively, as disclosed herein, and variant forms thereof lacking a signal peptide, an extracellular domain, a transmembrane domain or any other fragment of a full length native sequence Ihh, DefA5 or DefA6 polypeptide such as those referenced herein. Such variant polypeptides include, for instance, polypeptides wherein one or more amino acid residues are added, or deleted, at the N- or C-terminus of the full-length native amino acid sequence. In a specific aspect, such variant polypeptides will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence Ihh, DefA5 or DefA6 polypeptide sequence polypeptide, as disclosed herein, and variant forms thereof lacking a signal peptide, an extracellular domain, or any other fragment of a full length native sequence Ihh, DefA5 or DefA6 polypeptide such as those disclosed herein.

“Percent (%) amino acid sequence identity” with respect to an Ihh, DefA5 or a DefA6 polypeptide sequence identified herein is defined as the percentage of amino acid residues in a candidate sequence that are identical with the amino acid residues in the specific Ihh, DefA5 or DefA6 polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

As used herein “Ihh variant polynucleotide” or “Ihh variant nucleic acid sequence,” or “DefA5 variant polynucleotide” or “DefA5 variant nucleic acid sequence” or “DefA6 variant polynucleotide” or “DefA6 variant nucleic acid sequence” refers to a nucleic acid molecule which encodes an Ihh polypeptide, a DefA5 polypeptide or a DefA6 polypeptide, respectively, preferably active forms thereof, as defined herein, and which have at least about 80% nucleic acid sequence identity with a nucleotide acid sequence encoding a full-length native sequence Ihh, DefA5 or DefA6 polypeptide sequence identified herein, or any other fragment of the respective full-length Ihh, DefA5 or DefA6 polypeptide sequence as identified herein (such as those encoded by a nucleic acid that represents only a portion of the complete coding sequence for a full-length Ihh, DefA5 or DefA6 polypeptide). Ordinarily, such variant polynucleotides will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding the respective full-length native sequence Ihh, DefA5 or DefA6-polypeptide sequence or any other fragment of the respective full-length Ihh, DefA5 or DefA6 polypeptide sequence identified herein. Such variant polynucleotides do not encompass the native nucleotide sequence.

Ordinarily, such variant polynucleotides vary at least about 50 nucleotides in length from the native sequence polypeptide, alternatively the variance can be at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to an Ihh, DefA5 or DefA6 polypeptide-encoding nucleic acid sequences identified herein is defined as the percentage of nucleotides in a candidate sequence that are identical with the nucleotides in the Ihh, DefA5 or DefA6 nucleic acid sequence of interest, respectively, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program=s alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “REF-DNA”, wherein “REF-DNA” represents a hypothetical IHH-, DefA5- or DefA6-encoding nucleic acid sequence of interest, “Comparison DNA” represents the nucleotide sequence of a nucleic acid molecule against which the “REF-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, Ihh, DefA5 or DefA6 variant polynucleotides are nucleic acid molecules that encode Ihh, DefA5 or DefA6 polypeptide, respectively, and which are capable of hybridizing, preferably under stringent hybridization and wash conditions, to nucleotide sequences encoding a full-length Ihh, DefA5 or DefA6 polypeptide, respectively, as disclosed herein. Such variant polypeptides may be those that are encoded by such variant polynucleotides.

“Isolated”, when used to describe the various Ihh, DefA5 or DefA6 polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, such polypeptides will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Such isolated polypeptides includes the corresponding polypeptides in situ within recombinant cells, since at least one component of the Ihh, DefA5 or DefA6-polypeptide from its natural environment will not be present. Ordinarily, however, such isolated polypeptides will be prepared by at least one purification step.

An “isolated” Ihh, DefA5 or DefA6 polypeptide-encoding nucleic acid is a nucleic acid molecule that is identified and separated from at least one contaminant nucleic acid molecule with which it is ordinarily associated in the natural source of the polypeptide-encoding nucleic acid. Any of the above such isolated nucleic acid molecule is other than in the form or setting in which it is found in nature. Any such nucleic acid molecules therefore are distinguished from the specific polypeptide-encoding nucleic acid molecule as it exists in natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein “expression” as applied to gene expression, refers to transcription of a gene encoding a protein to produce mRNA as well as translation of the mRNA to produce the protein encoded by the gene. Thus, increased or decreased expression refers to increased or decreased transcription of a gene and/or increased or decreased translation of mRNA resulting from transcription.

The terms “inhibit”, “down-regulate”, “underexpress” and “reduce” are used interchangeably and mean that the expression of a gene, or level of RNA molecules or equivalent RNA molecules encoding one or more proteins or protein subunits, or activity of one or more proteins or protein subunits, is reduced relative to one or more controls, such as, for example, one or more positive and/or negative controls. The term “up-regulate” or “overexpress” is used to mean that the expression of a gene, or level of RNA molecules or equivalent RNA molecules encoding one or more proteins or protein subunits, or activity of one or more proteins or protein subunits, is elevated relative to one or more controls, such as, for example, one or more positive and/or negative controls. With regard to an RNA transcript, the terms “overexpress” and “underexpress” may be used to refer to the level of transcript determined by normalization to the level of reference mRNAs, which might be all transcripts detected in the test sample (or specimen) or a particular reference set of mRNAs.

The terms “differentially expressed gene,” “differential gene expression” and their synonyms, which are used interchangeably, refer to a gene whose expression is activated to a higher or lower level in a subject suffering from a disease, specifically an IBD, such as UC or CD, relative to its expression in a normal or control subject. The terms also include genes whose expression is activated to a higher or lower level at different stages of the same disease. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example. Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disease, specifically an IBD, or between various stages of the same disease. Differential expression includes both quantitative, as well as qualitative, differences in the temporal or cellular expression pattern in a gene or its expression products among, for example, normal and diseased cells, or among cells which have undergone different disease events or disease stages. For the purpose of this invention, “differential gene expression” is considered to be present when there is at least an about two-fold, preferably at least about four-fold, more preferably at least about six-fold, most preferably at least about ten-fold difference between the expression of a given gene in normal and diseased subjects, or in various stages of disease development in a diseased subject.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50 EC; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42 EC; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt=s solution, sonicated salmon sperm DNA (50 μg/ml), 0.1% SDS, and 10% dextran sulfate at 42 EC, with a 10 minute wash at 42 EC in 0.2×SSC (sodium chloride/sodium citrate) followed by a 10 minute high-stringency wash consisting of 0.1×SSC containing EDTA at 55 EC.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37 EC in a solution comprising: 20% formamide, 5×SSC (150 mM NaCl, 15 mM trisodium citrate), 50 mM sodium phosphate (pH 7.6), 5×Denhardt=s solution, 10% dextran sulfate, and 20 mg/ml denatured sheared salmon sperm DNA, followed by washing the filters in 1×SSC at about 37-50 EC. The ordinarily skilled artisan will recognize how to adjust the temperature, ionic strength, etc. as necessary to accommodate factors such as probe length and the like.

The term “epitope tagged” when used herein refers to a chimeric polypeptide comprising an Ihh, DefA5 or DefA6 polypeptide, or Ihh, DefA5 or DefA6 binding agent fused to a “tag polypeptide”. The tag polypeptide has enough residues to provide an epitope against which an antibody can be made, yet is short enough such that it does not interfere with the activity of the polypeptide to which it is fused. The tag polypeptide preferably also is sufficiently unique so that such antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of polypeptides which retain a biological and/or an immunological activity of native or naturally-occurring polypeptide, wherein “biological” activity refers to a biological function (either inhibitory or stimulatory) caused by a native or naturally-occurring polypeptide other than the ability to induce the production of an antibody against an antigenic epitope possessed by a native or naturally-occurring polypeptide, and an “immunological” activity refers to the ability to induce the production of an antibody against an antigenic epitope possessed by a native or naturally-occurring polypeptide. An active polypeptide, as used herein, is an antigen that is differentially expressed, either from a qualitative or quantitative perspective, in IBD tissue, relative to its expression on similar tissue that is not afflicted with IBD.

The term “antagonist” is used in the broadest sense, and includes any molecule that partially or fully blocks, inhibits, or neutralizes a biological activity of a native polypeptide disclosed herein. Suitable antagonist molecules specifically include antagonist antibodies or antibody fragments, fragments or amino acid sequence variants of native polypeptides, peptides, antisense oligonucleotides, and small organic molecules, as non limiting examples. Methods for identifying antagonists may comprise contacting such a polypeptide, including a cell expressing it, with a candidate agonist or antagonist molecule and measuring a detectable change in one or more biological activities normally associated with such polypeptide.

The term “modulate” is used herein to mean that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator.

The term “diagnosis” is used herein to refer to the identification of a molecular or pathological state, disease or condition, such as the identification of IBD. A diagnosis also refers to the process of identifying or determining the distinguishing characteristics of a disease including without limitation IBD, UC and/or Crohn's Disease. The process of diagnosing is sometimes also expressed as staging or disease classification based on severity or disease progression as well as on location (such as, for example, location within or along the gastrointestinal tract at which inflammation and/or altered gene expression is found).

The term “prognosis” is used herein to refer to the prediction of the likelihood of IBD development or progression, including autoimmune flare-ups and recurrences following surgery. Prognostic factors are those variables related to the natural history of IBD, which influence the recurrence rates and outcome of patients once they have developed IBD. Clinical parameters that may be associated with a worse prognosis include, for example, an abdominal mass or tenderness, skin rash, swollen joints, mouth ulcers, and borborygmus (gurgling or splashing sound over the intestine). Prognostic factors may be used to categorize patients into subgroups with different baseline recurrence risks.

The “pathology” of an IBD includes all phenomena that compromise the well-being of the patient. IBD pathology is primarily attributed to abnormal activation of the immune system in the intestines that can lead to chronic or acute inflammation in the absence of any known foreign antigen, and subsequent ulceration. Clinically, IBD is characterized by diverse manifestations often resulting in a chronic, unpredictable course. Bloody diarrhea and abdominal pain are often accompanied by fever and weight loss. Anemia is not uncommon, as is severe fatigue. Joint manifestations ranging from arthralgia to acute arthritis as well as abnormalities in liver function are commonly associated with IBD. During acute “attacks” of IBD, work and other normal activity are usually impossible, and often a patient is hospitalized.

The aetiology of these diseases is unknown and the initial lesion has not been clearly defined; however, patchy necrosis of the surface epithelium, focal accumulations of leukocytes adjacent to glandular crypts, and an increased number of intraepithelial lymphocytes and certain macrophage subsets have been described as putative early changes, especially in Crohn's disease.

The term “treatment” or “treating” or “alleviation” refers to both therapeutic treatment and prophylactic or preventative measures for IBD, wherein the object is to prevent or slow down (lessen) the targeted pathologic condition or disorder. Those in need of treatment include those already with an IBD as well as those prone to have an IBD or those in whom the IBD is to be prevented. Once the diagnosis of an IBD has been made by the methods disclosed herein, the goals of therapy are to induce and maintain a remission. Subjects in need of treatment or diagnosis include those already with aberrant Ihh, DefA5, and/or DefA6 expression as well as those prone to having or those in whom aberrant Ihh, DefA5 or DefA6 expression is to be prevented. A subject or mammal is successfully “treated” for aberrant Ihh, DefA5 or DefA6 expression if, according to the method of the present invention, after receiving a therapeutic amount of a therapeutic agent, the patient shows observable and/or measurable increase in Ihh expression toward normal levels of Ihh expression; a decrease in DefA5 and/or DefA6 expression toward normal levels of expression; or an improvement in the disease stage or status toward more normal gastrointestinal physiology, including without limitation reduction in gastrointestinal inflammation. Accordingly, an aspect of the invention is the detection of a therapeutic drug response in a mammal treated with a therapeutic agent for the treatment of IBD, wherein the method comprises determining Ihh, DefA5 and/or DefA6 expression in gastrointestinal tissue of a test mammal relative to a control and determining that the Ihh, DefA5 and/or DefA6 expression levels are within not significantly different from normal control expression levels. In one embodiment, a therapeutic response is determined when the levels of expression of Ihh and DefA6 of the mammal treated with a therapeutic agent are different (expression is more similar to normal control, i.e., Ihh levels are higher, and/or DefA5 and/or DefA6 levels are lower) than Ihh, DefA5 and/or DefA6 expression levels were in the mammal prior to treatment.

The above parameters for assessing successful treatment and improvement in the disease are readily measurable by routine procedures familiar to a physician. For IBD therapy, efficacy can be measured, for example, by assessing the time to disease progression (TTP) and/or determining the response rate (RR). Biopsies may be taken to assess gene expression and observe histopathology of gastrointestinal tissue from the patient. CT scans can also be done to look for spread to regions outside of the tumor or cancer. The invention described herein relating to the process of prognosing, diagnosing and/or treating involves the determination and evaluation of Ihh gene expression downregulation, and/or DefA5 and/or DefA6 gene expression upregulation or amplification.

“Mammal” for purposes of the treatment of, alleviating the symptoms of or diagnosis of a IBD refers to any animal classified as a mammal, including humans, domestic and farm animals, and zoo, sports, or pet animals, such as dogs, cats, cattle, horses, sheep, pigs, goats, rabbits, ferrets, etc. Preferably, the mammal is a human.

Various agents that are suitable for use as an “IBD therapeutic agent” are known to those of ordinary skill in the art. As described herein, such agents include without limitation, aminosalicylates, corticosteroids, and immunosuppressive agents.

The term “test sample” refers to a sample from a mammalian subject suspected of having an IBD, known to have an IBD, or known to be in remission from an IBD. The test sample may originate from various sources in the mammalian subject including, without limitation, blood, semen, serum, urine, bone marrow, mucosa, tissue, etc.

The term “control” or “control sample” refers a negative control in which a negative result is expected to help correlate a positive result in the test sample. Controls that are suitable for the present invention include, without limitation, a sample known to have normal levels of gene expression, a sample obtained from a mammalian subject known not to have an IBD, and a sample obtained from a mammalian subject known to be normal. A control may also be a sample obtained from a subject previously diagnosed and treated for an IBD who is currently in remission; and such a control is useful in determining any recurrence of an IBD in a subject who is in remission. In addition, the control may be a sample containing normal cells that have the same origin as cells contained in the test sample. Those of skill in the art will appreciate other controls suitable for use in the present invention.

The term “microarray” refers to an ordered arrangement of hybridizable array elements, preferably polynucleotide probes, on a substrate.

The term “polynucleotide,” when used in singular or plural, generally refers to any polyribonucleotide or polydeoxyribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs. The term includes DNAs (including cDNAs) and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The phrase “gene amplification” refers to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

In general, the term “marker” or “biomarker” or refers to an identifiable physical location on a chromosome, such as a restriction endonuclease recognition site or a gene, whose inheritance can be monitored. The marker may be an expressed region of a gene referred to as a “gene expression marker”, or some segment of DNA with no known coding function. An “IBD marker” as used herein refers to Ihh (SEQ ID NOS:1-2), DefA5 (SEQ ID NOS:3-4), and/or DefA6 (SEQ ID NOS:5-6).

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, typically: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide, followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In the context of the present invention, reference to “at least one,” “at least two,” “at least three,” of the genes listed in any particular gene set means any one or any and all combinations of the genes listed.

The terms “splicing” and “RNA splicing” are used interchangeably and refer to RNA processing that removes introns and joins exons to produce mature mRNA with continuous coding sequence that moves into the cytoplasm of an eukaryotic cell.

In theory, the term “exon” refers to any segment of an interrupted gene that is represented in the mature RNA product (B. Lewin. Genes IV Cell Press, Cambridge Mass. 1990). In theory the term “intron” refers to any segment of DNA that is transcribed but removed from within the transcript by splicing together the exons on either side of it. Operationally, exon sequences occur in the mRNA sequence of a gene as defined by Ref. SEQ ID numbers. Operationally, intron sequences are the intervening sequences within the genomic DNA of a gene, bracketed by exon sequences and having GT and AG splice consensus sequences at their 5′ and 3′ boundaries.

An “interfering RNA” or “small interfering RNA (siRNA)” is a double stranded RNA molecule usually less than about 30 nucleotides in length that reduces expression of a target gene. Interfering RNAs may be identified and synthesized using known methods (Shi Y., Trends in Genetics 19(1):9-12 (2003), WO/2003056012 and WO2003064621), and siRNA libraries are commercially available, for example from Dharmacon, Lafayette, Colo.

A “native sequence” polypeptide is one which has the same amino acid sequence as a polypeptide derived from nature, including naturally occurring or allelic variants. Such native sequence polypeptides can be isolated from nature or can be produced by recombinant or synthetic means. Thus, a native sequence polypeptide can have the amino acid sequence of naturally occurring human polypeptide, murine polypeptide, or polypeptide from any other mammalian species.

The term “antibody” herein is used in the broadest sense and specifically covers monoclonal antibodies, polyclonal antibodies, multispecific antibodies (e.g. bispecific antibodies), and antibody fragments, so long as they exhibit the desired biological activity. The present invention particularly contemplates antibodies against one or more of the IBD markers disclosed herein. Such antibodies may be referred to as “anti-IBD marker antibodies”. The term “antibody” specifically covers, for example, anti-Ihh, anti-DefA5 and/or anti-DefA6 monoclonal antibodies (including antagonist and neutralizing antibodies), anti-Ihh, anti-DefA5 and/or anti-DefA6 antibody compositions with polyepitopic specificity, polyclonal antibodies, single chain anti-Ihh, anti-DefA5 or anti-DefA6 antibodies, multispecific antibodies (e.g., bispecific) and antigen binding fragments (see below) of all of the above enumerated antibodies as long as they exhibit the desired biological or immunological activity. The term “immunoglobulin” (Ig) is used interchangeably with antibody herein.

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences.

For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies. A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol. 340(5):1073-1093 (2004); Fellouse, Proc. Nat. Acad. Sci. USA 101(34):12467-12472 (2004); and Lee et al. J. Immunol. Methods 284(1-2):119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Strict. Biol. 2:593-596 (1992).

Chimeric antibodies of interest herein include “primatized” antibodies comprising variable domain antigen-binding sequences derived from a non-human primate (e.g. Old World Monkey, Ape etc) and human constant region sequences, as well as “humanized” antibodies.

“Humanized” forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity.

An “intact antibody” herein is one which comprises two antigen binding regions, and an Fc region. Preferably, the intact antibody has a functional Fc region.

“Antibody fragments” comprise a portion of an intact antibody, preferably comprising the antigen binding region thereof. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragment(s).

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab=fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fab fragment also contains the constant domain of the light chain and the first constant domain (CH1) of the heavy chain. Fab=fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)₂ antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

The term “Fc region” herein is used to define a C-terminal region of an immunoglobulin heavy chain, including native sequence Fc regions and variant Fc regions. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

Unless indicated otherwise, herein the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called α, δ, ε, γ, and μ respectively. The subunit structures and three-dimensional configurations of different classes of immunoglobulins are well known.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain segments of the variable domains differ extensively in sequence among antibodies. The V domain mediates antigen binding and define specificity of a particular antibody for its particular antigen. However, the variability is not evenly distributed across the approximately 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). “Framework Region” or “FR” residues are those variable domain residues other than the hypervariable region residues as herein defined.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

As used herein “Ihh, DefA5, or DefA6 binding oligopeptide” or “Ihh, DefA5, or DefA6 binding polypeptide” is an oligopeptide that binds, preferably specifically, to an Ihh, DefA5 or DefA6 polypeptide, respectively, including a receptor (Patched (PTCH), for example), ligand or signaling component, or an Ihh, DefA5, or DefA6 binding portion or fragment thereof. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

An Ihh, DefA5 or DefA6 antagonist (e.g., antibody, polypeptide, oligopeptide or small molecule) “which binds” a target antigen of interest, e.g. Ihh, DefA5 or DefA6, respectively, is one that binds the target with sufficient affinity so as to be a useful diagnostic, prognostic and/or therapeutic agent in targeting a cell or tissue expressing the antigen, and does not significantly cross-react with other proteins. The extent of binding to a non-desired marker polypeptide will be less than about 10% of the binding to the particular desired target, as determinable by common techniques such as fluorescence activated cell sorting (FACS) analysis or radioimmunoprecipitation (RIA).

Moreover, the term “specific binding” or “specifically binds to” or is “specific for” a particular Ihh, DefA5 or DefA6-polypeptide or an epitope on a particular Ihh, DefA5 or DefA6 polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. In one embodiment, such terms refer to binding where a molecule binds to a particular polypeptide or epitope on a particular polypeptide without substantially binding to any other polypeptide or polypeptide epitope. Alternatively, such terms can be described by a molecule having a Kd for the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, or greater.

A gastrointestinal cell or tissue that “underexpresses” Ihh is a cell or tissue that exhibits decreased nucleic acid encoding Ihh, or a cell or tissue that under produces Ihh protein, compared to a normal gastrointestinal cell or tissue of the same tissue type. Such underexpression may result from genetic mutation or decreased transcription or translation. A gastrointestinal cell or tissue that “overexpresses” DefA5 and/or DefA6 is a cell or tissue that exhibits increased nucleic acid encoding DefA5 and/or DefA6, or a cell or tissue that over produces DefA5 and/or DefA6 protein, compared to a normal gastrointestinal cell or tissue of the same tissue type. Such overexpression may result from gene amplification or by increased transcription or translation. Various diagnostic or prognostic assays are known that measure altered expression levels resulting in increased or decreased levels of expressed protein at the cell surface or increased or decreased levels of secreted protein and include without limitation immunohistochemistry assay using anti-Ihh, anti-DefA5 and/or anti-DefA6 antibodies, FACS analysis, etc. Alternatively, the levels of Ihh-, DefA5- and/or DefA6-encoding nucleic acid or mRNA can be measured in the cell, e.g., via fluorescent in situ hybridization using a nucleic acid based probe corresponding to a hedgehog-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). Alternatively, hedgehog polypeptide underexpression or DefA5 or DefA6 overexpression is determinable by measuring shed antigen in feces or a biological fluid such as blood, serum or plasma, or in colon wash fluid (e.g. from a colonoscopy preparation) relative to a control, e.g, using antibody-based assays (see also, e.g., U.S. Pat. No. 4,933,294 issued Jun. 12, 1990; WO91/05264 published Apr. 18, 1991; U.S. Pat. No. 5,401,638 issued Mar. 28, 1995; and Sias et al., J. Immunol. Methods 132:73-80 (1990)). In addition to the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the therapeutic agent.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody, oligopeptide or other organic molecule so as to generate a “labeled” antibody, oligopeptide or other organic molecule. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” or “therapeutic agent” is a chemical compound useful in the treatment of a disorder or disease. Examples of chemotherapeutic or therapeutic agents for the treatment of IBD include, without limitation, anti-inflammatory drugs sulfasalazine and 5-aminosalisylic acid (5-ASA); metroidazole and ciprofloxacin are similar in efficacy to sulfasalazine and appear to be particularly useful for treating perianal disease; in more severe cases, corticosteroids are effective in treating active exacerbations and can even maintain remission; azathioprine, 6-mercaptopurine, and methotrexate have also shown success in patients who require chronic administration of cortico steroids; antidiarrheal drugs can also provide symptomatic relief in some patients; nutritional therapy or elemental diet can improve the nutritional status of patients and induce symtomatic improvement of acute disease; antibiotics are used in treating secondary small bowel bacterial overgrowth and in treatment of pyogenic complications. IBD chemotherapeutic agents further include biologicals and other agents as follows: anti-beta7 antibodies (see, for example, WO2006026759), anti-alpha4 antibodies (such as ANTEGEN®)), anti-TNF antibody (REMICADE®)) or non-protein compounds including without limitation 5-ASA compounds ASACOL®, PENTASA™, ROWASA™, COLAZAL™, and other compounds such as Purinethol and steroids such as prednisone. Examples of chemotherapeutic agents for the treatment of cancer include hydroxyureataxanes (such as paclitaxel and doxetaxel) and/or anthracycline antibiotics; alkylating agents such as thiotepa and CYTOXAN7 cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL7); beta-lapachone; lapachol; colchicines; betulinic acid; a camptothecin (including the synthetic analogue topotecan (HYCAMTIN₇), CPT-11 (irinotecan, CAMPTOSAR⁷), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as caimustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN₇ doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK₇ polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE₇, FILDESIN₇); dacarbazine; mannomustine; mitobronitol; mitolactol; pipobroman; gacytosine; arabinoside (“Ara-C”); thiotepa; taxoids, e.g., TAXOL₇ paclitaxel (Bristol-Myers Squibb Oncology, Princeton, N.J.), ABRAXANETM Cremophor-free, albumin-engineered nanoparticle formulation of paclitaxel (American Pharmaceutical Partners, Schaumberg, Ill.), and TAXOTERE₇ doxetaxel (Rhône-Poulenc Rorer, Antony, France); chloranbucil; gemcitabine (GEMZAR₇); 6-thioguanine; mercaptopurine; methotrexate; platinum analogs such as cisplatin and carboplatin; vinblastine (VELBAN₇); platinum; etoposide (VP-16); ifosfamide; mitoxantrone; vincristine (ONCOVIN₇); oxaliplatin; leucovovin; vinorelbine (NAVELBINE₇); novantrone; edatrexate; daunomycin; aminopterin; ibandronate; topoisomerase inhibitor RFS 2000; difluoromethylomithine (DMFO); retinoids such as retinoic acid; capecitabine (XELODA₇); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATINTM) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX₇ tamoxifen), EVISTA₇ raloxifene, droloxifene, 4-hydroxytamoxifen, trioxifene, keoxifene, LY117018, onapristone, and FARESTON₇ toremifene; anti-progesterones; estrogen receptor down-regulators (ERDs); agents that function to suppress or shut down the ovaries, for example, leutinizing hormone-releasing hormone (LHRH) agonists such as LUPRON₇ and ELIGARD₇ leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE₇ megestrol acetate, AROMASIN₇ exemestane, formestanie, fadrozole, RIVISOR₇ vorozole, FEMARA₇ letrozole, and ARIMIDEX₇ anastrozole. In addition, such definition of chemotherapeutic agents includes bisphosphonates such as clodronate (for example, BONEFOS₇ or OSTAC₇), DIDROCAL₇ etidronate, NE-58095, ZOMETA₇ zoledronic acid/zoledronate, FOSAMAX₇ alendronate, AREDIA₇ pamidronate, SKELID₇ tiludronate, or ACTONEL₇ risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE₇ vaccine and gene therapy vaccines, for example, ALLOVECTIN₇ vaccine, LEUVECTIN₇ vaccine, and VAXID₇ vaccine; LURTOTECAN₇ topoisomerase 1 inhibitor; ABARELIX₇ rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell, either in vitro or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes or hydroxyureataxanes (paclitaxel and docetaxel) are anticancer drugs both derived from the yew tree. Docetaxel (TAXOTERE₇, Rhone-Poulenc Rorer), derived from the European yew, is a semisynthetic analogue of paclitaxel (TAXOL₇, Bristol-Myers Squibb). These molecules promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

“Epithelia,” “epithelial” and “epithelium” refer to the cellular covering of internal and external body surfaces (cutaneous, mucous and serous), including the glands and other structures derived therefrom, e.g., corneal, esophageal, epidermal, and hair follicle epithelial cells. Other exemplary epithelial tissue includes: olfactory epithelium—the pseudostratified epithelium lining the olfactory region of the nasal cavity, and containing the receptors for the sense of smell; glandular epithelium—the epithelium composed of secreting cells squamous epithelium; squamous epithelium—the epithelium comprising one or more cell layers, the most superficial of which is comosed of flat, scalelike or platelike cells. Epithelium can also refer to transitional epithelium, like that which is characteristically found lining hollow organs that are subject to great mechanical change due to contraction and distention, e.g. tissue which represents a transition between stratified squamous and columnar epithelium.

The “growth state” of a cell refers to the rate of proliferation of the cell and/or the state of differentiation of the cell. An “altered growth state” is a growth state characterized by an abnormal rate of proliferation, e.g., a cell exhibiting an increased or decreased rate of proliferation relative to a normal cell.

The term “hedgehog” or “hedgehog polypeptide” (Hh) is used herein to refer generically to any of the mammalian homologs of the Drosophila hedgehog, i.e., sonic hedgehog (sHh), desert hedgehog (dHh) or Indian hedgehog (IHh). The term may be used to describe protein or nucleic acid.

The terms “hedgehog signaling pathway”, “hedgehog pathway” and “hedgehog signal transduction pathway” as used herein, interchangeably refer to the signaling cascade mediated by hedgehog and its receptors (e.g., patched, patched-2) and which results in changes of gene expression and other phenotypic changes typical of hedgehog activity. The hedgehog pathway may be activated in the absence of hedgehog through activation of a downstream component (e.g., overexpression of Smoothened or transfections with Smoothened or Patched mutants to result in constitutive activation with activate hedgehog signaling in the absence of hedgehog). The transcription factors of the Gli family are often used as markers or indicators of hedgehog pathway activation.

The term “Hh signaling component” refers to gene products that participate in the Hh signaling pathway. An Hh signaling component frequently materially or substantially affects the transmission of the Hh signal in cells or tissues, thereby affecting the downstream gene expression levels and/or other phenotypic changes associated with hedgehog pathway activation. Each Hh signaling component, depending on their biological function and effects on the final outcome of the downstream gene activation or expression, can be classified as either positive or negative regulators. A positive regulator is an Hh signaling component that positively affects the transmission of the Hh signal, i.e., stimulates downstream biological events when Hh is present. A negative regulator is an Hh signaling component that negative affects the transmission of the Hh signal, i.e. inhibits downstream biological events when Hh is present.

The word “label” when used herein refers to a compound or composition that is conjugated or fused directly or indirectly to a reagent such as a nucleic acid probe or an antibody and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which is detectable. The term is intended to encompass direct labeling of a probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “diabodies” refers to small antibody fragments with two antigen-binding sites, which fragments comprise a variable heavy domain (V_{H}) connected to a variable light domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “naked antibody” is an antibody that is not conjugated to a heterologous molecule, such as a small molecule or radiolabel.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step. The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant dmain of the heavy chain (CHI). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

An “affinity matured” antibody is one with one or more alterations in one or more hypervariable regions thereof which result an improvement in the affinity of the antibody for antigen, compared to a parent antibody which does not possess those alteration(s). Preferred affinity matured antibodies will have nanomolar or even picomolar affinities for the target antigen. Affinity matured antibodies are produced by procedures known in the art. Marks et al. Bio/Technology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

An “amino acid sequence variant” antibody herein is an antibody with an amino acid sequence which differs from a main species antibody. Ordinarily, amino acid sequence variants will possess at least about 70% homology with the main species antibody, and preferably, they will be at least about 80%, more preferably at least about 90% homologous with the main species antibody. The amino acid sequence variants possess substitutions, deletions, and/or additions at certain positions within or adjacent to the amino acid sequence of the main species antibody. Examples of amino acid sequence variants herein include an acidic variant (e.g. deamidated antibody variant), a basic variant, an antibody with an amino-terminal leader extension (e.g. VHS-) on one or two light chains thereof, an antibody with a C-terminal lysine residue on one or two heavy chains thereof, etc., and includes combinations of variations to the amino acid sequences of heavy and/or light chains. The antibody variant of particular interest herein is the antibody comprising an amino-terminal leader extension on one or two light chains thereof, optionally further comprising other amino acid sequence and/or glycosylation differences relative to the main species antibody.

A “glycosylation variant” antibody herein is an antibody with one or more carbohydrate moieities attached thereto which differ from one or more carbohydrate moieties attached to a main species antibody. Examples of glycosylation variants herein include antibody with a G1 or G2 oligosaccharide structure, instead a G0 oligosaccharide structure, attached to an Fc region thereof, antibody with one or two carbohydrate moieties attached to one or two light chains thereof, antibody with no carbohydrate attached to one or two heavy chains of the antibody, etc., and combinations of glycosylation alterations.

Where the antibody has an Fc region, an oligosaccharide structure may be attached to one or two heavy chains of the antibody, e.g. at residue 299 (298, Eu numbering of residues). For pertuzumab, G0 was the predominant oligosaccharide structure, with other oligosaccharide structures such as G0-F, G-1, Man5, Man6, G1-1, G1(1-6), G1(1-3) and G2 being found in lesser amounts in the pertuzumab composition.

Unless indicated otherwise, a “G1 oligosaccharide structure” herein includes G-1, G1-1, G1(1-6) and G1(1-3) structures.

An “amino-terminal leader extension” herein refers to one or more amino acid residues of the amino-terminal leader sequence that are present at the amino-terminus of any one or more heavy or light chains of an antibody. An exemplary amino-terminal leader extension comprises or consists of three amino acid residues, VHS, present on one or both light chains of an antibody variant.

A “deamidated” antibody is one in which one or more asparagine residues thereof has been derivatized, e.g. to an aspartic acid, a succinimide, or an iso-aspartic acid.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN₇, polyethylene glycol (PEG), and PLURONICS₇.

By “solid phase” or “solid support” is meant a non-aqueous matrix to which a polypeptide, nucleic acid, antibody or Ihh, DefA5 and/or DefA6 binding agent of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes.

A “small molecule” or “small organic molecule” is defined herein to have a molecular weight below about 500 Daltons.

An “effective amount” of an antagonist agent is an amount sufficient to bring about a physiological effect, such as without limitation to inhibit, partially or entirely, function of gene or its encoded protein. An “effective amount” may be determined empirically and in a routine manner, in relation to this purpose.

The term “therapeutically effective amount” refers to an antagonist or other drug effective to “treat” a disease or disorder in a subject or mammal. In the case of IBD, the therapeutically effective amount of the drug will restore aberrant Ihh, DefA5 and/or DefA6-expression to normal physiological levels; reduce gastrointestinal inflammation; reduce the number of gastrointestinal lesions; and/or relieve to some extent one or more of the symptoms associated with IBD, UC and/or CD. See the definition herein of “treating”

A “growth inhibitory amount” of an antagonist is an amount capable of inhibiting the growth of a cell, especially tumor, e.g., cancer cell, either in vitro or in vivo. For purposes of inhibiting neoplastic cell growth, such an amount may be determined empirically and in a routine manner.

A “cytotoxic amount” of an antagonist is an amount capable of causing the destruction of a cell, especially a proliferating cell, e.g., cancer cell, either in vitro or in vivo. For purposes of inhibiting neoplastic cell growth may be determined empirically and in a routine manner.

The terms “level of expression” or “expression level” are used interchangeably and generally refer to the amount of a polynucleotide or an amino acid product or protein in a biological sample. “Expression” generally refers to the process by which gene-encoded information is converted into the structures present and operating in the cell. Therefore, according to the invention “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

The term “overexpression” as used herein, refers to cellular gene expression levels of a tissue that is higher than the normal expression levels for that tissue. The term “underexpression” as used herein, refers to cellular gene expression levels of a tissue that is lower than the normal expression levels for that tissue. In either case, the higher or lower expression is significantly different from normal expression under controlled conditions of the study.

A “control” includes a sample obtained for use in determining base-line or normal expression or activity in a mammal that is not experiencing IBD. Accordingly, a control sample may be obtained by a number of means including from cells not affected by inflammation and/or IBD, UC or CD (as determined by standard techniques); non-IBD cells or tissue e.g., from cells of a subject not experiencing IBD; from subjects not having an IBD, Crohn's disease, or ulcerative colitis disorder; from subjects not suspected of being at risk for an IBD, CD or UC; from cells or cell lines derived from such subjects; or from tissues or cells of an IBD patient where such tissues or cells are normal and not affected by inflammation and/or IBD, UC or CD. A control also includes a previously established standard. Accordingly, any test or assay conducted according to the invention may be compared with the established standard and it may not be necessary to obtain a control sample for comparison each time.

The term “proliferating” and “proliferation” refer to a cellor cells undergoing mitosis.

Table 1 provides a computer algorithm for determining sequence identity.

TABLE 1
/*
*
* C-C increased from 12 to 15
* Z is average of EQ
* B is average of ND
* match with stop is _M; stop-stop = 0; J (joker) match = 0
*/

| | | |
|---|---|---|
| #define _M | −8 | /* value of a match with a stop */ |

int _day[26][26] = {
/* A B C D E F G H I J K L M N O P Q R S T U V W X Y Z */

| | |
|---|---|
| /* A */ | { 2, 0,−2, 0, 0,−4, 1,−1,−1, 0,−1,−2,−1, 0,_M, 1, 0,−2, 1, 1, 0, 0,−6, 0,−3, 0}, |
| /* B */ | { 0, 3,−4, 3, 2,−5, 0, 1,−2, 0, 0,−3,−2, 2,_M,−1, 1, 0, 0, 0, 0,−2,−5, 0,−3, 1}, |
| /* C */ | {−2,−4,15,−5,−5,−4,−3,−3,−2, 0,−5,−6,−5,−4,_M,−3,−5,−4, 0,−2, 0,−2,−8, 0, 0,−5}, |
| /* D */ | { 0, 3,−5, 4, 3,−6, 1, 1,−2, 0, 0,−4,−3, 2,_M,−1, 2,−1, 0, 0, 0,−2,−7, 0,−4, 2}, |
| /* E */ | { 0, 2,−5, 3, 4,−5, 0, 1,−2, 0, 0,−3,−2, 1,_M,−1, 2,−1, 0, 0, 0,−2,−7, 0,−4, 3}, |
| /* F */ | {−4,−5,−4,−6,−5, 9,−5,−2, 1, 0,−5, 2, 0,−4,_M,−5,−5,−4,−3,−3, 0,−1, 0, 0, 7,−5}, |
| /* G */ | { 1, 0,−3, 1, 0,−5, 5,−2,−3, 0,−2,−4,−3, 0,_M,−1,−1,−3, 1, 0, 0,−1,−7, 0,−5, 0}, |
| /* H */ | {−1, 1,−3, 1, 1,−2,−2, 6,−2, 0, 0,−2,−2, 2,_M, 0, 3, 2,−1,−1, 0,−2,−3, 0, 0, 2}, |
| /* I */ | {−1,−2,−2,−2,−2, 1,−3,−2, 5, 0,−2, 2, 2,−2,_M,−2,−2,−2,−1, 0, 0, 4,−5, 0,−1,−2}, |
| /* J */ | { 0, 0, 0, 0, 0, 0, 0, 0, 0, 0, 0, 0, 0, 0,_M, 0, 0, 0, 0, 0, 0, 0, 0, 0, 0, 0}, |
| /* K */ | {−1, 0,−5, 0, 0,−5,−2, 0,−2, 0, 5,−3, 0, 1,_M,−1, 1, 3, 0, 0, 0,−2,−3, 0,−4, 0}, |
| /* L */ | {−2,−3,−6,−4,−3, 2,−4,−2, 2, 0,−3, 6, 4,−3,_M,−3,−2,−3,−3,−1, 0, 2,−2, 0,−1,−2}, |
| /* M */ | {−1,−2,−5,−3,−2, 0,−3,−2, 2, 0, 0, 4, 6,−2,_M,−2,−1, 0,−2,−1, 0, 2,−4, 0,−2,−1}, |
| /* N */ | { 0, 2,−4, 2, 1,−4, 0, 2,−2, 0, 1,−3,−2, 2,_M,−1, 1, 0, 1, 0, 0,−2,−4, 0,−2, 1}, |
| /* O */ | {_M,_M,_M,_M,_M,_M,_M,_M,_M,_M,_M,_M,_M,_M, |

0,_M,_M,_M,_M,_M,_M,_M,_M,_M,_M,_M},

| | |
|---|---|
| /* P */ | { 1,−1,−3,−1,−1,−5,−1, 0,−2, 0,−1,−3,−2,−1,_M, 6, 0, 0, 1, 0, 0,−1,−6, 0,−5, 0}, |
| /* Q */ | { 0, 1,−5, 2, 2,−5,−1, 3,−2, 0, 1,−2,−1, 1,_M, 0, 4, 1,−1,−1, 0,−2,−5, 0,−4, 3}, |
| /* R */ | {−2, 0,−4,−1,−1,−4,−3, 2,−2, 0, 3,−3, 0, 0,_M, 0, 1, 6, 0,−1, 0,−2, 2, 0,−4, 0}, |
| /* S */ | { 1, 0, 0, 0, 0,−3, 1,−1,−1, 0, 0,−3,−2, 1,_M, 1,−1, 0, 2, 1, 0,−1,−2, 0,−3, 0}, |
| /* T */ | { 1, 0,−2, 0, 0,−3, 0,−1, 0, 0, 0,−1,−1, 0,_M, 0,−1,−1, 1, 3, 0, 0,−5, 0,−3, 0}, |
| /* U */ | { 0, 0, 0, 0, 0, 0, 0, 0, 0, 0, 0, 0, 0, 0,_M, 0, 0, 0, 0, 0, 0, 0, 0, 0, 0, 0}, |
| /* V */ | { 0,−2,−2,−2,−2,−1,−1,−2, 4, 0,−2, 2, 2,−2,_M,−1,−2,−2,−1, 0, 0, 4,−6, 0,−2,−2}, |
| /* W */ | {−6,−5,−8,−7,−7, 0,−7,−3,−5, 0,−3,−2,−4,−4,_M,−6,−5, 2,−2,−5, 0,−6,17, 0, 0,−6}, |
| /* X */ | { 0, 0, 0, 0, 0, 0, 0, 0, 0, 0, 0, 0, 0, 0,_M, 0, 0, 0, 0, 0, 0, 0, 0, 0, 0, 0}, |
| /* Y */ | {−3,−3, 0,−4,−4, 7,−5, 0,−1, 0,−4,−1,−2,−2,_M,−5,−4,−4,−3,−3, 0,−2, 0, 0,10,−4}, |
| /* Z */ | { 0, 1,−5, 2, 3,−5, 0, 2,−2, 0, 0,−2,−1, 1,_M, 0, 3, 0, 0, 0, 0,−2,−6, 0,−4, 4} |
| }; | |
| /* | |
| */ | |

#include <stdio.h>
#include <ctype.h>

| | | | |
|---|---|---|---|
| #define | MAXJMP | 16 | /* max jumps in a diag */ |
| #define | MAXGAP | 24 | /* don't continue to penalize gaps larger than this */ |
| #define | JMPS | 1024 | /* max jmps in an path */ |
| #define | MX | 4 | /* save if there's at least MX−1 bases since last jmp */ |
| #define | DMAT | 3 | /* value of matching bases */ |
| #define | DMIS | 0 | /* penalty for mismatched bases */ |
| #define | DINS0 | 8 | /* penalty for a gap */ |
| #define | DINS1 | 1 | /* penalty per base */ |
| #define | PINS0 | 8 | /* penalty for a gap */ |
| #define | PINS1 | 4 | /* penalty per residue */ |

struct jmp {

| | short | n[MAXJMP]; | /* size of jmp (neg for dely) */ |
|---|---|---|---|
| | unsigned short | x[MAXJMP]; | /* base no. of jmp in seq x */ |
| | | | /* limits seq to 2\hat{ }16 −1 */ |
| }; | | | |
| struct diag { | | | |
| | int | score; | /* score at last jmp */ |
| | long | offset; | /* offset of prev block */ |
| | short | ijmp; | /* current jmp index */ |
| | struct jmp | jp; | /* list of jmps */ |

};
struct path {

| | | | |
|---|---|---|---|
| | int | spc; | /* number of leading spaces */ |
| | short | n[JMPS]; | /* size of jmp (gap) */ |
| | int | x[JMPS]; | /* loc of jmp (last elem before gap) */ |
| }; | | | |

| | | |
|---|---|---|
| char | *ofile; | /* output file name */ |
| char | *namex[2]; | /* seq names: getseqs( ) */ |
| char | *prog; | /* prog name for err msgs */ |

| | | |
|---|---|---|
| char | *seqx[2]; | /* seqs: getseqs( ) */ |

| int | | dmax; | /* best diag: nw( ) */ |
|---|---|---|---|
| int | | dmax0; | /* final diag */ |
| int | | dna; | /* set if dna: main( ) */ |
| int | | endgaps; | /* set if penalizing end gaps */ |
| int | | gapx, gapy; | /* total gaps in seqs */ |
| int | | len0, len1; | /* seq lens */ |
| int | | ngapx, ngapy; | /* total size of gaps */ |
| int | | smax; | /* max score: nw( ) */ |
| int | | *xbm; | /* bitmap for matching */ |
| long | | offset; | /* current offset in jmp file */ |
| struct | diag | *dx; | /* holds diagonals */ |
| struct | path | pp[2]; | /* holds path for seqs */ |

| | |
|---|---|
| char | *calloc( ), *malloc( ), *index( ), *strcpy( ); |
| char | *getseq( ), *g_calloc( ); |

/* Needleman-Wunsch alignment program
*
* usage: progs file1 file2
* where file1 and file2 are two dna or two protein sequences.
* The sequences can be in upper- or lower-case an may contain ambiguity
* Any lines beginning with ‘;’, ‘>’ or ‘<’ are ignored
* Max file length is 65535 (limited by unsigned short x in the jmp struct)
* A sequence with ⅓ or more of its elements ACGTU is assumed to be DNA
* Output is in the file “align.out”
*
* The program may create a tmp file in /tmp to hold info about traceback.
* Original version developed under BSD 4.3 on a vax 8650
*/
#include “nw.h”
#include “day.h”

| | |
|---|---|
| static | _dbval[26] = { |
| | 1,14,2,13,0,0,4,11,0,0,12,0,3,15,0,0,0,5,6,8,8,7,9,0,10,0 |
| }; | |
| static | _pbval[26] = { |
| | 1, 2\|(1<<(‘D’-‘A’))\|(1<<(‘N’-‘A’)), 4, 8, 16, 32, 64, |
| | 128, 256, 0xFFFFFFF, 1<<10, 1<<11, 1<<12, 1<<13, 1<<14, |
| | 1<<15, 1<<16, 1<<17, 1<<18, 1<<19, 1<<20, 1<<21, 1<<22, |
| | 1<<23, 1<<24, 1<<25\|(1<<(‘E’-‘A’))\|(1<<(‘Q’-‘A’)) |
| }; | |

| | |
|---|---|
| main(ac, av) | main |

| | |
|---|---|
| int | ac; |
| char | *av[ ]; |

{

prog = av[0];
if (ac != 3) {

fprintf(stderr,“usage: %s file1 file2\n”, prog);
fprintf(stderr,“where file1 and file2 are two dna or two protein sequences.\n”);
fprintf(stderr,“The sequences can be in upper- or lower-case\n”);
fprintf(stderr,“Any lines beginning with ‘;’ or ‘<’ are ignored\n”);
fprintf(stderr,“Output is in the file \”align.out\“\n”);
exit(1);

| } | |
|---|---|
| namex[0] = av[1]; | |
| namex[1] = av[2]; | |
| seqx[0] = getseq(namex[0], &len0); | |
| seqx[1] = getseq(namex[1], &len1); | |
| xbm = (dna)? _dbval : _pbval; | |
| endgaps = 0; | /* 1 to penalize endgaps */ |
| ofile = “align.out”; | /* output file */ |

| | |
|---|---|
| nw( ); | /* fill in the matrix, get the possible jmps */ |
| readjmps( ); | /* get the actual jmps */ |
| print( ); | /* print stats, alignment */ |
| cleanup(0); | /* unlink any tmp files */ |

}
/* do the alignment, return best score: main( )
* dna: values in Fitch and Smith, PNAS, 80, 1382-1386, 1983
* pro: PAM 250 values
* When scores are equal, we prefer mismatches to any gap, prefer
* a new gap to extending an ongoing gap, and prefer a gap in seqx
* to a gap in seq y.
*/

| | |
|---|---|
| nw( ) | nw |
| { | |

| | | |
|---|---|---|
| char | *px, *py; | /* seqs and ptrs */ |

| | | |
|---|---|---|
| int | *ndely, *dely; | /* keep track of dely */ |
| int | ndelx, delx; | /* keep track of delx */ |
| int | *tmp; | /* for swapping row0, row1 */ |
| int | mis; | /* score for each type */ |
| int | ins0, ins1; | /* insertion penalties */ |
| register | id; | /* diagonal index */ |
| register | ij; | /* jmp index */ |
| register | *col0, *col1; | /* score for curr, last row */ |
| register | xx, yy; | /* index into seqs */ |

dx = (struct diag *)g_calloc(“to get diags”, len0+len1+1, sizeof(struct diag));
ndely = (int *)g_calloc(“to get ndely”, len1+1, sizeof(int));
dely = (int *)g_calloc(“to get dely”, len1+1, sizeof(int));
col0 = (int *)g_calloc(“to get col0”, len1+1, sizeof(int));
col1 = (int *)g_calloc(“to get col1”, len1+1, sizeof(int));
ins0 = (dna)? DINS0 : PINS0;
ins1 = (dna)? DINS1 : PINS1;
smax = −10000;
if (endgaps) {

for (col0[0] = dely[0] = −ins0, yy = 1; yy <= len1; yy++) {

col0[yy] = dely[yy] = col0[yy−1] − ins1;
ndely[yy] = yy;

| | |
|---|---|
| } | |
| col0[0] = 0; | /* Waterman Bull Math Biol 84 */ |

}
else

for (yy = 1; yy <= len1; yy++)

dely[yy] = −ins0;

/* fill in match matrix
*/
for (px = seqx[0], xx = 1; xx <= len0; px++, xx++) {

/* initialize first entry in col
*/
if (endgaps) {

| if (xx == 1) | |
|---|---|
| | col1[0] = delx = −(ins0+ins1); |
| else | |
| | col1[0] = delx = col0[0] − ins1; |

| | |
|---|---|
| | ndelx = xx; |
| } | |
| else { | |
| | col1[0] = 0; |
| | delx = −ins0; |
| | ndelx = 0; |
| } | |

...nw

for (py = seqx[1], yy = 1; yy <= len1; py++, yy++) {

| mis = col0[yy−1]; | |
|---|---|
| if (dna) | |
| | mis += (xbm[*px−‘A’]&xbm[*py−‘A’])? DMAT : DMIS; |
| else | |
| | mis += _day[*px−‘A’][*py−‘A’]; |

/* update penalty for del in x seq;
* favor new del over ongong del
* ignore MAXGAP if weighting endgaps
*/
if (endgaps || ndely[yy] < MAXGAP) {

if (col0[yy] − ins0 >= dely[yy]) {

dely[yy] = col0[yy] − (ins0+ins1);
ndely[yy] = 1;

} else {

dely[yy] −= ins1;
ndely[yy]++;

}

} else {

if (col0[yy] − (ins0+ins1) >= dely[yy]) {

dely[yy] = col0[yy] − (ins0+ins1);
ndely[yy] = 1;

} else

ndely[yy]++;

}
/* update penalty for del in y seq;
* favor new del over ongong del
*/
if (endgaps || ndelx < MAXGAP) {

if (col1[yy−1] − ins0 >= delx) {

delx = col1[yy−1] − (ins0+ins1);
ndelx = 1;

} else {

delx −= ins1;
ndelx++;

}

} else {

if (col1[yy−1] − (ins0+ins1) >= delx) {

delx = col1[yy−1] − (ins0+ins1);
ndelx = 1;

} else

ndelx++;

| | } |
|---|---|
| | /* pick the maximum score; we're favoring |
| | * mis over any del and delx over dely |
| | */ |
| | id = xx − yy + len1 − 1; |
| ...nw | |
| | if (mis >= delx && mis >= dely[yy]) |

col1[yy] = mis;

else if (delx >= dely[yy]) {

col1[yy] = delx;
ij = dx[id].ijmp;
if (dx[id].jp.n[0] && (!dna || (ndelx >= MAXJMP
&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] = ndelx;
dx[id].jp.x[ij] = xx;
dx[id].score = delx;

}
else {

col1[yy] = dely[yy];
ij = dx[id].ijmp;

if (dx[id].jp.n[0] && (!dna || (ndely[yy] >= MAXJMP

&& xx > dx[id].jp.x[ij]+MX) || mis > dx[id].score+DINS0)) {

dx[id].ijmp++;
if (++ij >= MAXJMP) {

writejmps(id);
ij = dx[id].ijmp = 0;
dx[id].offset = offset;
offset += sizeof(struct jmp) + sizeof(offset);

}

}
dx[id].jp.n[ij] = −ndely[yy];
dx[id].jp.x[ij] = xx;
dx[id].score = dely[yy];

}
if (xx == len0 && yy < len1) {

/* last col
*/
if (endgaps)

col1[yy] −= ins0+ins1*(len1−yy);

if (col1[yy] > smax) {

smax = col1[yy];
dmax = id;

}

}

}
if (endgaps && xx < len0)

col1[yy−1] −= ins0+ins1*(len0−xx);

if (col1[yy−1] > smax) {

smax = col1[yy−1];
dmax = id;

}
tmp = col0; col0 = col1; col1 = tmp;

}
(void) free((char *)ndely);
(void) free((char *)dely);
(void) free((char *)col0);
(void) free((char *)col1);

}
/*
*
* print( ) -- only routine visible outside this module
*
* static:
* getmat( ) -- trace back best path, count matches: print( )
* pr_align( ) -- print alignment of described in array p[ ]: print( )
* dumpblock( ) -- dump a block of lines with numbers, stars: pr_align( )
* nums( ) -- put out a number line: dumpblock( )
* putline( ) -- put out a line (name, [num], seq, [num]): dumpblock( )
* stars( ) - -put a line of stars: dumpblock( )
* stripname( ) -- strip any path and prefix from a seqname
*/
#include “nw.h”

| | | |
|---|---|---|
| #define SPC | 3 | |
| #define P_LINE | 256 | /* maximum output line */ |
| #define P_SPC | 3 | /* space between name or num and seq */ |

| | | |
|---|---|---|
| extern | _day[26][26]; | |
| int | olen; | /* set output line length */ |
| FILE | *fx; | /* output file */ |

| | |
|---|---|
| print( ) | print |
| { | |

| | | |
|---|---|---|
| int | lx, ly, firstgap, lastgap; | /* overlap */ |

if ((fx = fopen(ofile, “w”)) == 0) {

fprintf(stderr,“%s: can't write %s\n”, prog, ofile);
cleanup(1);

| } | |
|---|---|
| fprintf(fx, “<first sequence: %s (length = %d)\n”, namex[0], len0); | |
| fprintf(fx, “<second sequence: %s (length = %d)\n”, namex[1], len1); | |
| olen = 60; | |
| lx = len0; | |
| ly = len1; | |
| firstgap = lastgap = 0; | |
| if (dmax < len1 − 1) { | /* leading gap in x */ |

pp[0].spc = firstgap = len1 − dmax − 1;
ly −= pp[0].spc;

| | |
|---|---|
| } | |
| else if (dmax > len1 − 1) { | /* leading gap in y */ |

pp[1].spc = firstgap = dmax − (len1 − 1);
lx −= pp[1].spc;

| | |
|---|---|
| } | |
| if (dmax0 < len0 − 1) { | /* trailing gap in x */ |

lastgap = len0 − dmax0 −1;
lx −= lastgap;

| | |
|---|---|
| } | |
| else if (dmax0 > len0 − 1) { | /* trailing gap in y */ |

lastgap = dmax0 − (len0 − 1);
ly −= lastgap;

}
getmat(lx, ly, firstgap, lastgap);
pr_align( );

| | |
|---|---|
| } | |
| /* | |
| * trace back the best path, count matches | |
| */ | |
| static | |
| getmat(lx, ly, firstgap, lastgap) | getmat |

| | | |
|---|---|---|
| int | lx, ly; | /* “core” (minus endgaps) */ |
| int | firstgap, lastgap; | /* leading trailing overlap */ |

{

| | |
|---|---|
| int | nm, i0, i1, siz0, siz1; |
| char | outx[32]; |
| double | pct; |
| register | n0, n1; |
| register char | *p0, *p1; |

/* get total matches, score
*/
i0 = i1 = siz0 = siz1 = 0;
p0 = seqx[0] + pp[1].spc;
p1 = seqx[1] + pp[0].spc;
n0 = pp[1].spc + 1;
n1 = pp[0].spc + 1;
nm = 0;
while ( *p0 && *p1 ) {

if (siz0) {

p1++;
n1++;
siz0−−;

}
else if (siz1) {

p0++;
n0++;
siz1−−;

}
else {

if (xbm[*p0−‘A’]&xbm[*p1−‘A’])

nm++;

if (n0++ == pp[0].x[i0])

siz0 = pp[0].n[i0++];

if (n1++ == pp[1].x[i1])

siz1 = pp[1].n[i1++];

p0++;
p1++;

}

}
/* pct homology:
* if penalizing endgaps, base is the shorter seq
* else, knock off overhangs and take shorter core
*/
if (endgaps)

lx = (len0 < len1)? len0 : len1;

else

lx = (lx < ly)? lx : ly;

pct = 100.*(double)nm/(double)lx;
fprintf(fx, “\n”);
fprintf(fx, “<%d match%s in an overlap of %d: %.2f percent similarity\n”,

nm, (nm == 1)? “” : “es”, lx, pct);

| | |
|---|---|
| fprintf(fx, “<gaps in first sequence: %d”, gapx); | ...getmat |
| if (gapx) { | |

(void) sprintf(outx, “ (%d %s%s)”,

ngapx, (dna)? “base”:“residue”, (ngapx == 1)? “”:“s”);

fprintf(fx,“%s”, outx);

fprintf(fx, “, gaps in second sequence: %d”, gapy);
if (gapy) {

(void) sprintf(outx, “ (%d %s%s)”,

ngapy, (dna)? “base”:“residue”, (ngapy == 1)? “”:“s”);

fprintf(fx,“%s”, outx);

}
if (dna)

fprintf(fx,
“\n<score: %d (match = %d, mismatch = %d, gap penalty = %d + %d per

base)\n”,

smax, DMAT, DMIS, DINS0, DINS1);

else

fprintf(fx,
“\n<score: %d (Dayhoff PAM 250 matrix, gap penalty = %d + %d per residue)\n”,
smax, PINS0, PINS1);

if (endgaps)

fprintf(fx,
“<endgaps penalized. left endgap: %d %s%s, right endgap: %d %s%s\n”,
firstgap, (dna)? “base” : “residue”, (firstgap == 1)? “” : “s”,
lastgap, (dna)? “base” : “residue”, (lastgap == 1)? “” : “s”);

else

| | fprintf(fx, “<endgaps not penalized\n”); | |
|---|---|---|
| } | | |
| static | nm; | /* matches in core -- for checking */ |
| static | lmax; | /* lengths of stripped file names */ |
| static | ij[2]; | /* jmp index for a path */ |
| static | nc[2]; | /* number at start of current line */ |
| static | ni[2]; | /* current elem number -- for gapping */ |
| static | siz[2]; | |
| static char | *ps[2]; | /* ptr to current element */ |
| static char | *po[2]; | /* ptr to next output char slot */ |
| static char | out[2][P_LINE]; | /* output line */ |
| static char | star[P_LINE]; | /* set by stars( ) */ |

| | |
|---|---|
| /* | |
| * print alignment of described in struct path pp[ ] | |
| */ | |
| static | |
| pr_align( ) | pr_align |
| { | |

| | | |
|---|---|---|
| int | nn; | /* char count */ |
| int | more; | |
| register | i; | |

for (i = 0, lmax = 0; i < 2; i++) {

nn = stripname(namex[i]);
if (nn > lmax)

lmax = nn;

nc[i] = 1;
ni[i] = 1;
siz[i] = ij[i] = 0;
ps[i] = seqx[i];
po[i] = out[i];

| | |
|---|---|
| } | |
| for (nn = nm = 0, more = 1; more; ) { | ...pr_align |

for (i = more = 0; i < 2; i++) {

/*
* do we have more of this sequence?
*/
if (!*ps[i])

continue;

| | |
|---|---|
| more++; | |
| if (pp[i].spc) { | /* leading space */ |

*po[i]++ = ‘ ’;
pp[i].spc−−;

| | |
|---|---|
| } | |
| else if (siz[i]) { | /* in a gap */ |

*po[i]++ = ‘-’;
siz[i]−−;

| | |
|---|---|
| } | |
| else { | /* we're putting a seq element |
| | */ |

*po[i] = *ps[i];
if (islower(*ps[i]))

*ps[i] = toupper(*ps[i]);

po[i]++;
ps[i]++;
/*
* are we at next gap for this seq?
*/
if (ni[i] == pp[i].x[ij[i]]) {

/*
* we need to merge all gaps
* at this location
*/
siz[i] = pp[i].n[ij[i]++];
while (ni[i] == pp[i].x[ij[i]])

siz[i] += pp[i].n[ij[i]++];

}
ni[i]++;

}

}
if (++nn == olen || !more && nn) {

dumpblock( );
for (i = 0; i < 2; i++)

po[i] = out[i];

nn = 0;

}

}

| | |
|---|---|
| } | |
| /* | |
| * dump a block of lines, including numbers, stars: pr_align( ) | |
| */ | |
| static | |
| dumpblock( ) | dumpblock |
| { | |

register i;
for (i = 0; i < 2; i++)

*po[i]−− = ‘\0’;

...dumpblock

(void) putc(‘\n’, fx);
for (i = 0; i < 2; i++) {

if (*out[i] && (*out[i] != ‘ ’ || *(po[i]) != ‘ ’)) {

if (i == 0)

nums(i);

if (i == 0 && *out[1])

stars( );

putline(i);
if (i == 0 && *out[1])

fprintf(fx, star);

if (i == 1)

nums(i);

}

}

}
/*
* put out a number line: dumpblock( )
*/
static

| | |
|---|---|
| nums(ix) | nums |

| | | |
|---|---|---|
| int | ix; | /* index in out[ ] holding seq line */ |

{

| | |
|---|---|
| char | nline[P_LINE]; |
| register | i, j; |
| register char | *pn, *px, *py; |

for (pn = nline, i = 0; i < lmax+P_SPC; i++, pn++)

*pn = ‘ ’;

for (i = nc[ix], py = out[ix]; *py; py++, pn++) {

if (*py == ‘ ’ || *py == ‘-’)

*pn = ‘ ’;

else {

if (i%10 == 0 || (i == 1 && nc[ix] != 1)) {

j = (i < 0)? −i : i;
for (px = pn; j; j /= 10, px−−)

*px = j%10 + ‘0’;

if (i < 0)

*px = ‘-’;

}
else

*pn = ‘ ’;

i++;

}

}
*pn = ‘\0’;
nc[ix] = i;
for (pn = nline; *pn; pn++)

(void) putc(*pn, fx);

(void) putc(‘\n’, fx);

| | |
|---|---|
| } | |
| /* | |
| * put out a line (name, [num], seq, [num]): dumpblock( ) | |
| */ | |
| static | |
| putline(ix) | putline |

| | |
|---|---|
| int | ix; |

| | |
|---|---|
| { | |
| | ...putline |

| | |
|---|---|
| int | i; |
| register char | *px; |

for (px = namex[ix], i = 0; *px && *px != ‘:’; px++, i++)

(void) putc(*px, fx);

for (; i < lmax+P_SPC; i++)

(void) putc(‘ ’, fx);

/* these count from 1:
* ni[ ] is current element (from 1)
* nc[ ] is number at start of current line
*/
for (px = out[ix]; *px; px++)

(void) putc(*px&0x7F, fx);

(void) putc(‘\n’, fx);

| | |
|---|---|
| } | |
| /* | |
| * put a line of stars (seqs always in out[0], out[1]): dumpblock( ) | |
| */ | |
| static | |
| stars( ) | stars |
| { | |

| | |
|---|---|
| int | i; |
| register char | *p0, *p1, cx, *px; |

if (!*out[0] || (*out[0] == ‘ ’ && *(po[0]) == ‘ ’) ||
!*out[1] || (*out[1] == ‘ ’ && *(po[1]) == ‘ ’))

return;

px = star;
for (i = lmax+P_SPC; i; i−−)

*px++ = ‘ ’;

for (p0 = out[0], p1 = out[1]; *p0 && *p1; p0++, p1++) {

if (isalpha(*p0) && isalpha(*p1)) {

if (xbm[*p0−‘A’]&xbm[*p1−‘A’]) {

cx = ‘*’;
nm++;

}
else if (!dna && _day[*p0−‘A’][*p1−‘A’] > 0)

cx = ‘.’;

else

cx = ‘ ’;

}
else

cx = ‘ ’;

*px++ = cx;

}
*px++ = ‘\n’;
*px = ‘\0’;

| | |
|---|---|
| } | |
| /* | |
| * strip path or prefix from pn, return len: pr_align( ) | |
| */ | |
| static | |
| stripname(pn) | stripname |

| | | | |
|---|---|---|---|
| | char | *pn; | /* file name (may be path) */ |
| { | | | |

| | |
|---|---|
| register char | *px, *py; |

py = 0;
for (px = pn; *px; px++)

if (*px == ‘/’)

py = px + 1;

if (py)

(void) strcpy(pn, py);

return(strlen(pn));

}
/*
* cleanup( ) -- cleanup any tmp file
* getseq( ) -- read in seq, set dna, len, maxlen
* g_calloc( ) -- calloc( ) with error checkin
* readjmps( ) -- get the good jmps, from tmp file if necessary
* writejmps( ) -- write a filled array of jmps to a tmp file: nw( )
*/
#include “nw.h”
#include <sys/file.h>

| | | |
|---|---|---|
| char | *jname = “/tmp/homgXXXXXX”; | /* tmp file for jmps */ |
| FILE | *fj; | |

| | | |
|---|---|---|
| int | cleanup( ); | /* cleanup tmp file */ |
| long | lseek( ); | |

| | |
|---|---|
| /* | |
| * remove any tmp file if we blow | |
| */ | |
| cleanup(i) | cleanup |

| | | |
|---|---|---|
| | int | i; |
| { | | |
| | if (fj) | |
| | | (void) unlink(jname); |
| | exit(i); | |

}
/*
* read, return ptr to seq, set dna, len, maxlen
* skip lines starting with ‘;’, ‘<’, or ‘>’
* seq in upper or lower case
*/

| | |
|---|---|
| char | * |

| | |
|---|---|
| getseq(file, len) | getseq |

| | | |
|---|---|---|
| char | *file; | /* file name */ |
| int | *len; | /* seq len */ |

{

| | |
|---|---|
| char | line[1024], *pseq; |
| register char | *px, *py; |
| int | natgc, tlen; |
| FILE | *fp; |

if ((fp = fopen(file,“r”)) == 0) {

fprintf(stderr,“%s: can't read %s\n”, prog, file);
exit(1);

}
tlen = natgc = 0;
while (fgets(line, 1024, fp)) {

if (*line == ‘;’ || *line == ‘<’ || *line == ‘>’)

continue;

for (px = line; *px != ‘\n’; px++)

if (isupper(*px) || islower(*px))

tlen++;

}
if ((pseq = malloc((unsigned)(tlen+6))) == 0) {

fprintf(stderr,“%s: malloc( ) failed to get %d bytes for %s\n”, prog, tlen+6, file);
exit(1);

}
pseq[0] = pseq[1] = pseq[2] = pseq[3] = ‘\0’;

...getseq

py = pseq + 4;
*len = tlen;
rewind(fp);
while (fgets(line, 1024, fp)) {

if (*line == ‘;’ || *line == ‘<’ || *line == ‘>’)

continue;

for (px = line; *px != ‘\n’; px++) {

if (isupper(*px))

*py++ = *px;

else if (islower(*px))

*py++ = toupper(*px);

if (index(“ATGCU”,*(py−1)))

natgc++;

}

| | } |
|---|---|
| | *py++ = ‘\0’; |
| | *py = ‘\0’; |
| | (void) fclose(fp); |
| | dna = natgc > (tlen/3); |
| | return(pseq+4); |
| } | |
| char | * |

| | |
|---|---|
| g_calloc(msg, nx, sz) | g_calloc |

| | | |
|---|---|---|
| char | *msg; | /* program, calling routine */ |
| int | nx, sz; | /* number and size of elements */ |

{

| | |
|---|---|
| char | *px, *calloc( ); |

if ((px = calloc((unsigned)nx, (unsigned)sz)) == 0) {

if (*msg) {

fprintf(stderr, “%s: g_calloc( ) failed %s (n=%d, sz=%d)\n”, prog, msg,

nx, sz);

exit(1);

}

}
return(px);

}
/*
* get final jmps from dx[ ] or tmp file, set pp[ ], reset dmax: main( )
*/

| | |
|---|---|
| readjmps( ) | readjmps |
| { | |

| | |
|---|---|
| int | fd = −1; |
| int | siz, i0, i1; |

| | |
|---|---|
| register | i, j, xx; |

if (fj) {

(void) fclose(fj);
if ((fd = open(jname, O_RDONLY, 0)) < 0) {

fprintf(stderr, “%s: can't open( ) %s\n”, prog, jname);
cleanup(1);

}

}
for (i = i0 = i1 = 0, dmax0 = dmax, xx = len0; ; i++) {

while (1) {

for (j = dx[dmax].ijmp; j >= 0 && dx[dmax].jp.x[j] >= xx; j−−)

;

...readjmps

if (j < 0 && dx[dmax].offset && fj) {

(void) lseek(fd, dx[dmax].offset, 0);
(void) read(fd, (char *)&dx[dmax].jp, sizeof(struct jmp));
(void) read(fd, (char *)&dx[dmax].offset, sizeof(dx[dmax].offset));
dx[dmax].ijmp = MAXJMP−1;

}
else

break;

}
if (i >= JMPS) {

fprintf(stderr, “%s: too many gaps in alignment\n”, prog);
cleanup(1);

}
if (j >= 0) {

| siz = dx[dmax].jp.n[j]; | |
|---|---|
| xx = dx[dmax].jp.x[j]; | |
| dmax += siz; | |
| if (siz < 0) { | /* gap in second seq */ |

pp[1].n[i1] = −siz;
xx += siz;
/* id = xx − yy + len1 − 1
*/
pp[1].x[i1] = xx − dmax + len1 − 1;
gapy++;
ngapy −= siz;

/* ignore MAXGAP when doing endgaps */

siz = (−siz < MAXGAP || endgaps)? −siz : MAXGAP;
i1++;

| | |
|---|---|
| } | |
| else if (siz > 0) { | /* gap in first seq */ |

pp[0].n[i0] = siz;
pp[0].x[i0] = xx;
gapx++;
ngapx += siz;

/* ignore MAXGAP when doing endgaps */

siz = (siz < MAXGAP || endgaps)? siz : MAXGAP;
i0++;

}

}
else

break;

}
/* reverse the order of jmps
*/
for (j = 0, i0−−; j < i0; j++, i0−−) {

i = pp[0].n[j]; pp[0].n[j] = pp[0].n[i0]; pp[0].n[i0] = i;
i = pp[0].x[j]; pp[0].x[j] = pp[0].x[i0]; pp[0].x[i0] = i;

}
for (j = 0, i1−−; j < i1; j++, i1−−) {

i = pp[1].n[j]; pp[1].n[j] = pp[1].n[i1]; pp[1].n[i1] = i;
i = pp[1].x[j]; pp[1].x[j] = pp[1].x[i1]; pp[1].x[i1] = i;

}
if (fd >= 0)

(void) close(fd);

if (fj) {

(void) unlink(jname);
fj = 0;
offset = 0;

}

/*
* write a filled jmp struct offset of the prev one (if any): nw( )
*/

| | |
|---|---|
| writejmps(ix) | writejmps |

| | |
|---|---|
| int | ix; |

{

| | |
|---|---|
| char | *mktemp( ); |

if (!fj) {

if (mktemp(jname) < 0) {

fprintf(stderr, “%s: can't mktemp( ) %s\n”, prog, jname);
cleanup(1);

}
if ((fj = fopen(jname, “w”)) == 0) {

fprintf(stderr, “%s: can't write %s\n”, prog, jname);
exit(1);

}

}
(void) fwrite((char *)&dx[ix].jp, sizeof(struct jmp), 1, fj);
(void) fwrite((char *)&dx[ix].offset, sizeof(dx[ix].offset), 1, fj);

}

TABLE 2

| | | |
|---|---|---|
| Reference | XXXXXXXXXXXXXXX | (Length = 15 amino |
| | | acids) |
| Comparison | XXXXXYYYYYYY | (Length = 12 amino |
| Protein | | acids) |

% amino acid sequence identity = (the number of identically matching amino acid residues between the two polypeptide sequences as determined by ALIGN-2) divided by (the total number of amino acid residues of the reference polypeptide) = 5 divided by 15 = 33.3%

TABLE 3

| | | |
|---|---|---|
| Reference | XXXXXXXXXX | (Length = 10 amino |
| | | acids) |
| Comparison | XXXXXYYYYYYZZYZ | (Length = 15 amino |
| Protein | | acids) |

% amino acid sequence identity = (the number of identically matching amino acid residues between the two polypeptide sequences as determined by ALIGN-2) divided by (the total number of amino acid residues of the reference polypeptide) = 5 divided by 10 = 50%

TABLE 4

| | | |
|---|---|---|
| Reference-DNA | NNNNNNNNNNNNNN | (Length = 14 |
| | | nucleotides) |
| Comparison DNA | NNNNNNLLLLLLLLLL | (Length = 16 |
| | | nucleotides) |

% nucleic acid sequence identity = (the number of identically matching nucleotides between the two nucleic acid sequences as determined by ALIGN-2) divided by (the total number of nucleotides of the reference-DNA nucleic acid sequence) = 6 divided by 14 = 42.9%

TABLE 5

| | | |
|---|---|---|
| Reference-DNA | NNNNNNNNNNNN | (Length = 12 |
| | | nucleotides) |
| Comparison DNA | NNNNLLLVV | (Length = 9 |
| | | nucleotides) |

% nucleic acid sequence identity = (the number of identically matching nucleotides between the two nucleic acid sequences as determined by ALIGN-2) divided by (the total number of nucleotides of the reference-DNA nucleic acid sequence) = 4 divided by 12 = 33.3%

### B.1 General Description of the Invention

The practice of the present invention will employ, unless otherwise indicated, conventional techniques of molecular biology (including recombinant techniques), microbiology, cell biology, and biochemistry, which are within the skill of the art. Such techniques are explained fully in the literature, such as, “Molecular Cloning: A Laboratory Manual”, 2^{nd} edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology”, 4^{th} edition (D. M. Weir & C. C. Blackwell, eds., Blackwell Science Inc., 1987); “Gene Transfer Vectors for Mammalian Cells” (J. M. Miller & M. P. Calos, eds., 1987); “Current Protocols in Molecular Biology” (F. M. Ausubel et al., eds., 1987); and “PCR: The Polymerase Chain Reaction”, (Mullis et al., eds., 1994).

The detection or diagnosis of IBD is currently obtained by various classification systems that rely on a number of variables observed in a patient. The present invention is based on the identification of genes that are associated with IBD. Accordingly, the expression levels of such genes can serve as diagnostic markers to identify patients with IBD. As described in the Examples, the differential expression of Ihh, DEFA5, and DEFA6 genes in IBD patients has been observed. Thus, according to the present invention, these genes have been identified as differentially expressed in IBD.

a. Biomarkers of the Invention

The present invention provides gene expression markers or biomarkers for IBD: Ihh, DEFA5, and DEFA6. In one embodiment of the present invention, a preferred set of IBD markers identified by microarray analysis, includes markers that are upregulated in an IBD. Preferably, the set of upregulated markers includes Ihh (SEQ ID NOS:1-2), DEFA5 (SEQ ID NOS:3-4), and DEFA6 (SEQ ID NO:5-6). A panel of biomarkers as described herein may include one of, more than one of, or all of these markers. These markers, singly or in any combination, are preferred for use in prognostic and diagnostic assays of the present invention. The IBD markers of the present invention are differentially expressed genes. A differential level of expression of one or more markers in a test sample from a mammalian subject relative to a control can determined from the level of RNA transcripts or expression products detected by one or more of the methods described in further detail below.

Based on evidence of differential expression of RNA transcripts in normal cells and cells from a mammalian subject having IBD, the present invention provides gene markers for IBD. The IBD markers and associated information provided by the present invention allow physicians to make more intelligent treatment decisions, and to customize the treatment of IBD to the needs of individual patients, thereby maximizing the benefit of treatment and minimizing the exposure of patients to unnecessary treatments, which do not provide any significant benefits and often carry serious risks due to toxic side-effects.

Multi-analyte gene expression tests can measure the expression level of one or more genes involved in each of several relevant physiologic processes or component cellular characteristics. In some instances the predictive power of the test, and therefore its utility, can be improved by using the expression values obtained for individual genes to calculate a score which is more highly correlated with outcome than is the expression value of the individual genes. For example, the calculation of a quantitative score (recurrence score) that predicts the likelihood of recurrence in estrogen receptor-positive, node-negative breast cancer is describe in U.S. patent application (Publication Number 20050048542). The equation used to calculate such a recurrence score may group genes in order to maximize the predictive value of the recurrence score. The grouping of genes may be performed at least in part based on knowledge of their contribution to physiologic functions or component cellular characteristics such as discussed above. The formation of groups, in addition, can facilitate the mathematical weighting of the contribution of various expression values to the recurrence score. The weighting of a gene group representing a physiological process or component cellular characteristic can reflect the contribution of that process or characteristic to the pathology of the IBD and clinical outcome. Accordingly, in an important aspect, the present invention also provides specific groups of the genes identified herein, that together are more reliable and powerful predictors of outcome than the individual genes or random combinations of the genes identified.

In addition, based on the determination of a recurrence score, one can choose to partition patients into subgroups at any particular value(s) of the recurrence score, where all patients with values in a given range can be classified as belonging to a particular risk group. Thus, the values chosen will define subgroups of patients with respectively greater or lesser risk.

The utility of a gene marker in predicting the development or progression of an IBD may not be unique to that marker. An alternative marker having a expression pattern that is closely similar to a particular test marker may be substituted for or used in addition to a test marker and have little impact on the overall predictive utility of the test. The closely similar expression patterns of two genes may result from involvement of both genes in a particular process and/or being under common regulatory control. The present invention specifically includes and contemplates the use of such substitute genes or gene sets in the methods of the present invention.

The markers and associated information provided by the present invention predicting the development and/or progression of an IBD also have utility in screening patients for inclusion in clinical trials that test the efficacy of drug compounds for the treatment of patients with IBD.

The markers and associated information provided by the present invention predicting the presence, development and/or progression of an IBD are useful as criterion for determining whether IBD treatment is appropriate. For example, IBD treatment may be appropriate where the results of the test indicate that an IBD marker is differentially expressed in a test sample from an individual relative to a control sample. The individual may be an individual not known to have an IBD, an individual known to have an IBD, an individual previously diagnosed with an IBD undergoing treatment for the IBD, or an individual previously diagnosed with an IBD and having had surgery to address the IBD. In addition, the present invention contemplates methods of treating an IBD. As described below, the diagnostic methods of the present invention may further comprise the step of administering an IBD therapeutic agent to the mammalian subject that provided the test sample in which the differential expression of one or more IBD markers was observed relative to a control. Such methods of treatment would therefore comprise (a) determining the presence of an IBD in a mammalian subject, and (b) administering an IBD therapeutic agent to the mammalian subject.

In another embodiment, the IBD markers and associated information are used to design or produce a reagent that modulates the level or activity of the gene's transcript or its expression product. Said reagents may include but are not limited to an antisense RNA, a small inhibitory RNA (siRNA), a ribozyme, a monoclonal or polyclonal antibody. In a further embodiment, said gene or its transcript, or more particularly, an expression product of said transcript is used in an (screening) assay to identify a drug compound, wherein said drug compounds is used in the development of a drug to treat an IBD.

In various embodiments of the inventions, various technological approaches described below are available for determination of expression levels of the disclosed genes. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

b. Diagnostic Methods of the Invention

The present invention provides methods of detecting or diagnosing an IBD in a mammalian subject based on differential expression of an IBD marker. In one embodiment, the methods comprise the use of a panel of IBD markers that may include one or more of Ihh, DEFA5, and DEFA6.

It is further contemplated that use of therapeutic agents for IBD may be specifically targeted to disorders where the affected tissue and/or cells exhibit reduced Ihh expression, and/or increased DefA5 and/or DefA6 expression relative to control. Accordingly, it is contemplated that the detection of reduced Ihh gene expression, and/or increased DefA5 and/or DefA6 gene expression expression may be used as a powerful predictive tool to identify tissues and disorders that will particularly benefit from treatment with a therapeutic agent, including a chemotherapeutic agent, useful in ameliorating IBD, UC and/or CD in a human patient.

In preferred embodiments, Ihh, DefA5 and/or DefA6 expression levels are detected, either by direct detection of the transcript or by detection of protein levels or activity. Transcripts may be detected using any of a wide range of techniques that depend primarily on hybrization or probes to the Ihh, DefA5 and/or DefA6 transcripts or to cDNAs synthesized therefrom. Well known techniques include Northern blotting, reverse-transcriptase PCR and microarray analysis of transcript levels. Methods for detecting Ihh, DefA5 and/or DefA6 protein levels include Western blotting, immunoprecipitation, two-dimensional polyacrylatmide gel electrophoresis (2D SDS-PAGE—preferably compared against a standard wherein the position of the Ihh, DefA5 and/or DefA6 proteins has been determined), and mass spectroscopy. Mass spectroscopy may be coupled with a series of purification steps to allow high-throughput identification of many different protein levels in a particular sample. Mass spectroscopy and 2D SDS-PAGE can also be used to identify post-transcriptional modifications to proteins including proteolytic events, ubiquitination, phosphorylation, lipid modification, etc. Ihh, DefA5 and/or DefA6 activity may also be assessed by analyzing binding to substrate DNA or in vitro transcriptional activiaton of target promoters. Gel shift assay, DNA footprinting assays and DNA-protein crosslinking assays are all methods that may be used to assess the presence of a protein capable of binding to Gli binding sites on DNA. J. Mol. Med. 77(6):459-68 (1999); Cell 100(4): 423-34 (2000); Development 127(19): 4923-4301 (2000).

In certain embodiments, Ihh, DefA5 and/or DefA6 transcript levels are measured, and diseased or disordered tissues showing significantly low Ihh levels and/or significantly high levels of DefA5 and/or DefA6 relative to control are treated with an IBD therapeutic compound. Accordingly, Ihh, DefA5 and/or DefA6 expression levels are a powerful diagnostic measure for determining whether a patient is experiencing IBD and whether that patient should receive an IBD therapeutic agent.

In another embodiment, the panels of the present invention may include an IBD marker that is overexpressed in an active IBD relative to a control, underexpressed in an active IBD relative to a control, or IBD markers that are both overexpressed and underexpressed in an active IBD relative to a control. In another embodiment, the panels of the present invention may include an IBD marker that is overexpressed in an inactive IBD relative to a control, underexpressed in an inactive IBD relative to a control, or IBD markers that are both overexpressed and underexpressed in an inactive IBD relative to a control. In a preferred embodiment, the active IBD is CD. In another preferred embodiment, the inactive IBD is CD.

In a preferred embodiment, the methods of diagnosing or detecting the presence of an IBD in a mammalian subject comprise determining a differential expression level of RNA transcripts or expression products thereof from a panel of IBD markers in a test sample obtained from the subject relative to the level of expression in a control, wherein the differential level of expression is indicative of the presence of an IBD in the subject from which the test sample was obtained. The differential expression in the test sample may be higher and/or lower relative to a control as discussed herein.

Differential expression or activity of one or more of the genes provided in the lists above, or the corresponding RNA molecules or encoded proteins in a biological sample obtained from the patient, relative to control, indicates the presence of an IBD in the patient. The control can, for example, be a gene, present in the same cell, which is known to be up-regulated (or down-regulated) in an IBD patient (positive control). Alternatively, or in addition, the control can be the expression level of the same gene in a normal cell of the same cell type (negative control). Expression levels can also be normalized, for example, to the expression levels of housekeeping genes, such as glyceraldehyde-3-phosphate-dehydrogenase (GAPDH) and/or β-actin, or to the expression levels of all genes in the sample tested. In one embodiment, expression of one or more of the above noted genes is deemed positive expression if it is at the median or above, e.g. compared to other samples of the same type. The median expression level can be determined essentially contemporaneously with measuring gene expression, or may have been determined previously. These and other methods are well known in the art, and are apparent to those skilled in the art.

Methods for identifying IBD patients are provided herein. Of this patient population, patients with an IBD can be identified by determining the expression level of one or more of the genes, the corresponding RNA molecules or encoded proteins in a biological sample comprising cells obtained from the patient. The biological sample can, for example, be a tissue biopsy as described herein.

The methods of the present invention concern IBD diagnostic assays, and imaging methodologies. In one embodiment, the assays are performed using antibodies as described herein. The invention also provides various immunological assays useful for the detection and quantification of proteins. These assays are performed within various immunological assay formats well known in the art, including but not limited to various types of radioimmunoassays, enzyme-linked immunosorbent assays (ELISA), enzyme-linked immunofluorescent assays (ELIFA), and the like. In addition, immunological imaging methods capable of detecting an IBD characterized by expression of a molecule described herein are also provided by the invention, including but not limited to radioscintigraphic imaging methods using labeled antibodies. Such assays are clinically useful in the detection, monitoring, diagnosis and prognosis of IBD characterized by expression of one or more molecules described herein.

Another aspect of the present invention relates to methods for identifying a cell that expresses a molecule described herein. The expression profile of a molecule(s) described herein make it a diagnostic marker for IBD. Accordingly, the status of the expression of the molecule(s) provides information useful for predicting a variety of factors including susceptibility to advanced stages of disease, rate of progression, and/or sudden and severe onset of symptoms in an active IBD or an inactive IBD, i.e. flare-ups.

In one embodiment, the present invention provides methods of detecting an IBD. A test sample from a mammalian subject and a control sample from a known normal mammal are each contacted with an anti-IBD marker antibody or a fragment thereof. The level of IBD marker expression is measured and a differential level of expression in the test sample relative to the control sample is indicative of an IBD in the mammalian subject from which the test sample was obtained. In some embodiments, the level of IBD marker expression in the test sample is determined to be higher than the level of expression in the control, wherein the higher level of expression indicates the presence of an IBD in the subject from which the test sample was obtained. In another embodiments, the level of IBD marker expression in the test sample is determined to be lower than the level of expression in the control, wherein the lower level of expression indicates the presence of an IBD in the subject from which the test sample was obtained.

In another embodiment, the IBD detected by the methods of the present invention is the recurrence or flareup of an IBD in the mammalian subject.

In preferred embodiments, the methods are employed to detect the flare-up of an IBD or a recurrence of an IBD in a mammalian subject previously determined to have an IBD who underwent treatment for the IBD, such as drug therapy or a surgical procedure. Following initial detection of an IBD, additional test samples may be obtained from the mammalian subject found to have an IBD. The additional sample may be obtained hours, days, weeks, or months after the initial sample was taken. Those of skill in the art will appreciate the appropriate schedule for obtaining such additional samples, which may include second, third, fourth, fifth, sixth, etc. test samples. The initial test sample and the additional sample (and alternately a control sample as described herein) are contacted with an anti-IBD marker antibody. The level of IBD marker expression is measured and a differential level of expression in the additional test sample as compared to the initial test sample is indicative of a flare-up in or a recurrence of an IBD in the mammalian subject from which the test sample was obtained.

In one aspect, the methods of the present invention are directed to a determining step. In one embodiment, the determining step comprises measuring the level of expression of one or more IBD markers in a test sample relative to a control. Typically, measuring the level of IBD marker expression, as described herein, involves analyzing a test sample for differential expression of an IBD marker relative to a control by performing one or more of the techniques described herein. The expression level data obtained from a test sample and a control are compared for differential levels of expression. In another embodiment, the determining step further comprises an examination of the test sample and control expression data to assess whether an IBD is present in the subject from which the test sample was obtained.

In some embodiments, the determining step comprises the use of a software program executed by a suitable processor for the purpose of (i) measuring the differential level of IBD marker expression in a test sample and a control; and/or (ii) analyzing the data obtained from measuring differential level of IBD marker expression in a test sample and a control. Suitable software and processors are well known in the art and are commercially available. The program may be embodied in software stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

Following the determining step, the measurement results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. In some embodiments, the assays will be performed or the assay results analyzed in a country or jurisdiction which differs from the country or jurisdiction to which the results or diagnoses are communicated.

In a preferred embodiment, a diagnosis, prediction and/or treatment recommendation based on the level of expression of one or more IBD markers disclosed herein measured in a test subject of having one or more of the IBD markers herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

The invention provides assays for detecting the differential expression of an IBD marker in tissues associated with the gastrointestinal tract including, without limitation, ascending colon tissue, descending colon tissue, sigmoid colon tissue, and terminal ileum tissue; as well expression in other biological samples such as serum, semen, bone, prostate, urine, cell preparations, and the like. Methods for detecting differential expression of an IBD marker are also well known and include, for example, immunoprecipitation, immunohistochemical analysis, Western blot analysis, molecular binding assays, ELISA, ELIFA and the like. For example, a method of detecting the differential expression of an IBD marker in a biological sample comprises first contacting the sample with an anti-IBD marker antibody, an IBD marker-reactive fragment thereof, or a recombinant protein containing an antigen-binding region of an anti-IBD marker antibody; and then detecting the binding of an IBD marker protein in the sample.

In various embodiments of the inventions, various technological approaches are available for determination of expression levels of the disclosed genes, including, without limitation, RT-PCR, microarrays, serial analysis of gene expression (SAGE) and Gene Expression Analysis by Massively Parallel Signature Sequencing (MPSS), which will be discussed in detail below. In particular embodiments, the expression level of each gene may be determined in relation to various features of the expression products of the gene including exons, introns, protein epitopes and protein activity. In other embodiments, the expression level of a gene may be inferred from analysis of the structure of the gene, for example from the analysis of the methylation pattern of gene's promoter(s).

To determine Ihh, DefA5 and/or DefA6 expression in IBD, various diagnostic assays are available. In one embodiment, Ihh nucleic acid or polypeptide underexpression and DefA5 and DefA6 nucleic acid or polypeptide overexpression may be analyzed by RT-PCR, in-situ hybridization, microarray analysis, and/or immunohistochemistry (IHC). Fresh, frozen and/or parafin embedded tissue sections from a gastrointestinal biopsy (such as from the colon or, more specifically, the sigmoid colon) from a mammal (such as without limitation a human) may be subjected to a RT-PCR, in situ hybridization, microarray analysis and/or IHC assay.

Alternatively, or additionally, FISH assays such as the INFORM₇ (sold by Ventana, Ariz.) or PATHVISION₇ (Vysis, Ill.) may be carried out on formalin-fixed, paraffin-embedded tissue to determine the extent (if any) of Ihh expression and/or downregulation, and/or DefA5 and/or DefA6 expression or upregulation in a tissue sample or biopsy.

Ihh, DefA5 and/or DefA6 expression may be evaluated using an in vivo diagnostic assay, e.g., by administering a molecule (such as an antibody, oligopeptide or organic molecule) which binds the Ihh, DefA5 or DefA6 nucleic acid or polypeptide to be detected and is tagged with a detectable label (e.g., a radioactive isotope or a fluorescent label) and externally scanning the patient for localization of the label.

c. Therapeutic Methods of the Invention

The present invention provides therapeutic methods of treating an IBD in a subject in need that comprise detecting the presence of an IBD in a mammalian subject by the diagnostic methods described herein and then administering to the mammalian subject an IBD therapeutic agent.

Anti-inflammatory drugs sulfasalazine and 5-aminosalisylic acid (5-ASA) are useful for treating mildly active colonic Crohn's disease and is commonly perscribed to maintain remission of the disease. Metroidazole and ciprofloxacin are similar in efficacy to sulfasalazine and appear to be particularly useful for treating perianal disease. In more severe cases, corticosteroids are effective in treating active exacerbations and can even maintain remission. Azathioprine and 6-mercaptopurine have also shown success in patients who require chronic administration of cortico steroids. It is also possible that these drugs may play a role in the long-term prophylaxis. Unfortunately, there can be a very long delay (up to six months) before onset of action in some patients. Antidiarrheal drugs can also provide symptomatic relief in some patients. Nutritional therapy or elemental diet can improve the nutritional status of patients and induce symtomatic improvement of acute disease, but it does not induce sustained clinical remissions. Antibiotics are used in treating secondary small bowel bacterial overgrowth and in treatment of pyogenic complications. Treatment for UC includes sulfasalazine and related salicylate-containing drugs for mild cases and corticosteroid drugs in severe cases. Topical administration of either salicylates or corticosteroids is sometimes effective, particularly when the disease is limited to the distal bowel, and is associated with decreased side effects compared with systemic use. Supportive measures such as administration of iron and antidiarrheal agents are sometimes indicated. Azathioprine, 6-mercaptopurine and methotrexate are sometimes also prescribed for use in refractory corticosteroid-dependent cases. Those of ordinary skill in the art will appreciate the various IBD therapeutic agents that may be suitable for use in the present invention (see St Clair Jones, Hospital Pharmacist, May 2006, Vol. 13; pages 161-166, hereby incorporated by reference in its entirety).

The present invention contemplates methods of IBD treatment in which one or more IBD therapeutic agents are administered to a subject in need. In one embodiment, the IBD therapeutic agent is one or more of an aminosalicylate, a corticosteroid, and an immunosuppressive agent. In a preferred embodiment, the aminosalicylate is one of sulfasalazine, olsalazine, mesalamine, balsalazide, and asacol. In another preferred embodiment, multiple aminosalicylates are co-administered, such as a combination of sulfasalazine and olsalazine. In other preferred embodiments, the corticosteroid may be budesonide, prednisone, prednisolone, methylprednisolone, 6-mercaptopurine (6-MP), azathioprine, methotrexate, and cyclosporin. In other preferred embodiments, the IBD therapeutic agent may an antibiotic, such as ciprofloxacin and/or metronidazole; or an antibody-based agent such as infliximab (Remicade®).

The least toxic IBD therapeutic agents which patients are typically treated with are the aminosalicylates. Sulfasalazine (Azulfidine), typically administered four times a day, consists of an active molecule of aminosalicylate (5-ASA) which is linked by an azo bond to a sulfapyridine. Anaerobic bacteria in the colon split the azo bond to release active 5-ASA. However, at least 20% of patients cannot tolerate sulfapyridine because it is associated with significant side-effects such as reversible sperm abnormalities, dyspepsia or allergic reactions to the sulpha component. These side effects are reduced in patients taking olsalazine. However, neither sulfasalazine nor olsalazine are effective for the treatment of small bowel inflammation. Other formulations of 5-ASA have been developed which are released in the small intestine (e.g. mesalamine and asacol). Normally it takes 6-8 weeks for 5-ASA therapy to show full efficacy. Patients who do not respond to 5-ASA therapy, or who have a more severe disease, are prescribed corticosteroids. However, this is a short term therapy and cannot be used as a maintenance therapy. Clinical remission is achieved with corticosteroids within 2-4 weeks, however the side effects are significant and include Cushing goldface, facial hair, severe mood swings and sleeplessness. The response to sulfasalazine and 5-aminosalicylate preparations is poor in CD, fair to mild in early ulcerative colitis and poor in severe UC. If these agents fail, powerful immunosuppressive agents such as cyclosporine, prednisone, 6-mercaptopurine or azathioprine (converted in the liver to 6-mercaptopurine) are typically tried. For CD patients, the use of corticosteroids and other immunosuppressives must be carefully monitored because of the high risk of intra-abdominal sepsis originating in the fistulas and abscesses common in this disease. Approximately 25% of IBD patients will require surgery (colectomy) during the course of the disease.

Treatment of an IBD may include a surgical procedure, including without limitation, a bowel resection, anastomosis, a colectomy, a proctocolectomy, and an ostomy, or any combination thereof.

In addition to pharmaceutical medicine and surgery, nonconventional treatments for IBD such as nutritional therapy have also been attempted. For example, Flexical®, a semi-elemental formula, has been shown to be as effective as the steroid prednisolone. Sanderson et al., Arch. Dis. Child. 51:123-7 (1987). However, semi-elemental formulas are relatively expensive and are typically unpalatable—thus their use has been restricted. Nutritional therapy incorporating whole proteins has also been attempted to alleviate the symptoms of IBD. Giafer et al., Lancet 335: 816-9 (1990). U.S. Pat. No. 5,461,033 describes the use of acidic casein isolated from bovine milk and TGF-2. Beattie et al., Aliment. Pharmacol. Ther. 8: 1-6 (1994) describes the use of casein in infant formula in children with IBD. U.S. Pat. No. 5,952,295 describes the use of casein in an enteric formulation for the treatment of IBD. However, while nutritional therapy is non-toxic, it is a palliative treatment and does not treat the underlying cause of the disease.

The present invention contemplates methods of IBD treatment, including for example, in vitro, ex vivo and in vivo therapeutic methods. The invention provides methods useful for treating an IBD in a subject in need upon the detection of an IBD disease state in the subject associated with the expression of one or more IBD markers disclosed herein, such as increased and/or decreased IBD marker expression. In one preferred embodiment, the method comprises (a) determining that a level of expression of (i) one or more RNA transcripts or expression products thereof of a gene shown as SEQ ID NO:1, SEQ ID NO:3, or SEQ ID NO:5, or (ii) one or more nucleic acids encoding a polypeptide shown as SEQ ID NO:2, SEQ ID NO:4, or SEQ ID NO:6 in a test sample obtained from said subject is higher and/or lower relative to a level of expression in a control, wherein said higher and/or lower level of expression is indicative of the presence of an IBD in the subject from which the test sample was obtained; and (b) administering to said subject an effective amount of an IBD therapeutic agent. The determining step (a) may comprise the measurement of the expression of multiple IBD markers.

The method of treatment comprises detecting the IBD and administering an effective amount of an IBD therapeutic agent to a subject in need of such treatment. In some embodiments, the IBD disease state is associated with an increased and/or decrease in expression of one or more IBD markers.

In one aspect, the invention provides methods for treating or preventing an IBD, the methods comprising detecting the presence of an IBD in a subject and administering an effective amount of an IBD therapeutic agent to the subject. It is understood that any suitable IBD therapeutic agent may be used in the methods of treatment, including aminosalicylates, corticosteroids, and immunosuppressive agents as discussed herein.

In any of the methods herein, one may administer to the subject or patient along with a single IBD therapeutic agent discussed herein an effective amount of a second medicament (where the single IBD therapeutic agent herein is a first medicament), which is another active agent that can treat the condition in the subject that requires treatment. For instance, an aminosalicylate may be co-administered with a corticosteroid, an immunsuppressive agent, or another aminosalicylate. The type of such second medicament depends on various factors, including the type of IBD, its severity, the condition and age of the patient, the type and dose of first medicament employed, etc.

Such treatments using first and second medicaments include combined administration (where the two or more agents are included in the same or separate formulations), and separate administration, in which case, administration of the first medicament can occur prior to, and/or following, administration of the second medicament. In general, such second medicaments may be administered within 48 hours after the first medicaments are administered, or within 24 hours, or within 12 hours, or within 3-12 hours after the first medicament, or may be administered over a pre-selected period of time, which is preferably about 1 to 2 days, about 2 to 3 days, about 3 to 4 days, about 4 to 5 days, about 5 to 6 days, or about 6 to 7 days.

The first and second medicaments can be administered concurrently, sequentially, or alternating with the first and second medicament or upon non-responsiveness with other therapy. Thus, the combined administration of a second medicament includes co-administration (concurrent administration), using separate formulations or a single pharmaceutical formulation, and consecutive administration in either order, wherein preferably there is a time period while both (or all) medicaments simultaneously exert their biological activities. All these second medicaments may be used in combination with each other or by themselves with the first medicament, so that the express “second medicament” as used herein does not mean it is the only medicament besides the first medicament, respectively. Thus, the second medicament need not be one medicament, but may constitute or comprise more than one such drug. These second medicaments as set forth herein are generally used in the same dosages and with administration routes as the first medicaments, or about from 1 to 99% of the dosages of the first medicaments. If such second medicaments are used at all, preferably, they are used in lower amounts than if the first medicament were not present, especially in subsequent dosings beyond the initial dosing with the first medicament, so as to eliminate or reduce side effects caused thereby.

Where the methods of the present invention comprise administering one or more IBD therapeutic agent to treat or prevent an IBD, it may be particularly desirable to combine the administering step with a surgical procedure that is also performed to treat or prevent the IBD. The IBD surgical procedures contemplated by the present invention include, without limitation, a bowel resection, anastomosis, a colectomy, a proctocolectomy, and an ostomy, or any combination thereof. For instance, an IBD therapeutic agent described herein may be combined with a colectomy in a treatment scheme, e.g. in treating an IBD. Such combined therapies include and separate administration, in which case, administration of the IBD therapeutic agent can occur prior to, and/or following, the surgical procedure.

Treatment with a combination of one or more IBD therapeutic agents; or a combination of one or more IBD therapeutic agents and a surgical procedure described herein preferably results in an improvement in the signs or symptoms of an IBD. For instance, such therapy may result in an improvement in the subject receiving the IBD therapeutic agent treatment regimen and a surgical procedure, as evidenced by a reduction in the severity of the pathology of the IBD.

The IBD therapeutic agent(s) is/are administered by any suitable means, including parenteral, subcutaneous, intraperitoneal, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic.

The IBD therapeutic agent(s) compositions administered according to the methods of the invention will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The first medicament(s) need not be, but is optionally formulated with one or more additional medicament(s) (e.g. second, third, fourth, etc. medicaments) described herein. The effective amount of such additional medicaments depends on the amount of the first medicament present in the formulation, the type of disorder or treatment, and other factors discussed above. These are generally used in the same dosages and with administration routes as used hereinbefore or about from 1 to 99% of the heretofore employed dosages.

For the prevention or treatment of an IBD, the appropriate dosage of an IBD therapeutic agent (when used alone or in combination with other agents) will depend on the type of disease to be treated, the type of IBD therapeutic agent(s), the severity and course of the disease, whether the IBD therapeutic agent is administered for preventive or therapeutic purposes, previous therapy, the patient's clinical history and response to the IBD therapeutic agent, and the discretion of the attending physician. The IBD therapeutic agent is suitably administered to the patient at one time or over a series of treatments. Depending on the type and severity of the disease, about 1 ug/kg to 15 mg/kg (e.g. 0.1 mg/kg-10 mg/kg) of IBD therapeutic agent is an initial candidate dosage for administration to the patient, whether, for example, by one or more separate administrations, or by continuous infusion. One typical daily dosage might range from about 1 ug/kg to 100 mg/kg or more, depending on the factors mentioned above. For repeated administrations over several days or longer, depending on the condition, the treatment is sustained until a desired suppression of disease symptoms occurs. One exemplary dosage of the IBD therapeutic agent would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, e.g. about six doses of the IBD therapeutic agent). An initial higher loading dose, followed by one or more lower doses may be administered. An exemplary dosing regimen comprises administering an initial loading dose of about 4 mg/kg, followed by a weekly maintenance dose of about 2 mg/kg of the IBD therapeutic agent. However, other dosage regimens may be useful. The progress of this therapy is easily monitored by conventional techniques and assays.

Currently, depending on the stage of the IBD, treatment involves one or a combination of the following therapies: surgery to remove affected bowel tissue, administration of therapeutic agents, including without limitation chemotherapy; dietary changes, and lifestyle management. Therapeutic agents or chemotherapeutic agents useful in the treatment of IBD are known in the art and representative therapeutic and chemotherapeutic agents are disclosed herein.

In particular, combination therapy with palictaxel and modified derivatives (see, e.g., EP0600517) is contemplated. The preceding antibody, polypeptide, oligopeptide or organic molecule will be administered with a therapeutically effective dose of the chemotherapeutic agent. In another embodiment, such antibody, polypeptide, oligopeptide or organic molecule is administered in conjunction with chemotherapy to enhance the activity and efficacy of the chemotherapeutic agent, e.g., paclitaxel. The Physicians=Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

Therapeutic agents or chemotherapeutic agents are administered to a human patient, in accord with known methods, such as intravenous administration, e.g., as a bolus or by continuous infusion over a period of time, by intracranial, intracerobrospinal, intra-articular, intrathecal, intravenous, intraarterial, subcutaneous, oral, topical, or inhalation routes.

Other therapeutic regimens may be combined with the administration of the foregoing therapeutic or chemotherapeutic agents for the treatment of IBD. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

In another embodiment, the therapeutic treatments include without limitation the combined administration of one or more therapeutic or chemotherapeutic agents, including co-administration of cocktails of different chemotherapeutic agents. Example chemotherapeutic agents have been provided previously. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

For the prevention or treatment of disease, the dosage and mode of administration will be chosen by the physician according to known criteria. The appropriate dosage will depend on the type of disease to be treated, the severity and course of the disease, whether administration is for preventive or therapeutic purposes, previous therapy (including) the patient's clinical history and response, and the discretion of the attending physician. The therapeutic agents may be suitably administered to the patient at one time or over a series of treatments. Administration may occur by intravenous infusion or by subcutaneous injections. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding an DefA5 or DefA6 antagonist or Ihh agonist is encompassed by the expression “administering a therapeutically effective amount of an antibody”. See, for example, WO96/07321 published Mar. 14, 1996 concerning the use of gene therapy to generate intracellular antibodies.

There are two major approaches to introducing such nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

### B.2. Gene Expression Profiling

In general, methods of gene expression profiling can be divided into two large groups: methods based on hybridization analysis of polynucleotides, and other methods based on biochemical detection or sequencing of polynucleotides. The most commonly used methods known in the art for the quantification of mRNA expression in a sample include northern blotting and in situ hybridization (Parker & Barnes, Methods in Molecular Biology 106:247-283 (1999)); RNAse protection assays (Hod, Biotechniques 13:852-854 (1992)); and reverse transcription polymerase chain reaction (RT-PCR) (Weis et al., Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Various methods for determining expression of mRNA or protein include, but are not limited to, gene expression profiling, polymerase chain reaction (PCR) including quantitative real time PCR (qRT-PCR), microarray analysis that can be performed by commercially available equipment, following manufacturer's protocols, such as by using the Affymetrix GenChip technology, serial analysis of gene expression (SAGE) (Velculescu et al., Science 270:484-487 (1995); and Velculescu et al., Cell 88:243-51 (1997)), MassARRAY, Gene Expression Analysis by Massively Parallel Signature Sequencing (MPSS) (Brenner et al., Nature Biotechnology 18:630-634 (2000)), proteomics, immunohistochemistry (IHC), etc. Preferably mRNA is quantified. Such mRNA analysis is preferably performed using the technique of polymerase chain reaction (PCR), or by microarray analysis. Where PCR is employed, a preferred form of PCR is quantitative real time PCR (qRT-PCR).

a. Reverse Transcriptase PCR(RT-PCR)

Of the techniques listed above, the most sensitive and most flexible quantitative method is RT-PCR, which can be used to compare mRNA levels in different sample populations, in normal and test sample tissues, to characterize patterns of gene expression, to discriminate between closely related mRNAs, and to analyze RNA structure.

The first step is the isolation of mRNA from a target sample. The starting material is typically total RNA isolated from colonic tissue biopsies. Thus, RNA can be isolated from a variety of tissues, including without limitation, the terminal ileum, the ascending colon, the descending colon, and the sigmoid colon. In addition, the colonic tissue from which a biopsy is obtained may be from an inflamed and/or a non-inflamed colonic area.

In one embodiment, the mRNA is obtained from a biopsy as defined above wherein the biopsy is obtained from the left colon or from the right colon. As used herein, the “left colon” refers to the sigmoideum and rectosigmoideum and the “right colon” refers to the cecum.

General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. Total RNA from tissue samples can be isolated using RNA Stat-60 (Tel-Test). RNA prepared from a biopsy can be isolated, for example, by cesium chloride density gradient centrifugation.

As RNA cannot serve as a template for PCR, the first step in gene expression profiling by RT-PCR is the reverse transcription of the RNA template into cDNA, followed by its exponential amplification in a PCR reaction. The two most commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. Thus, TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan® RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In a preferred embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and β-actin.

A more recent variation of the RT-PCR technique is the real time quantitative PCR, which measures PCR product accumulation through a dual-labeled fluorigenic probe (i.e., TaqMan® probe). Real time PCR is compatible both with quantitative competitive PCR, where internal competitor for each target sequence is used for normalization, and with quantitative comparative PCR using a normalization gene contained within the sample, or a housekeeping gene for RT-PCR. For further details see, e.g. Held et al., Genome Research 6:986-994 (1996).

According to one aspect of the present invention, PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T.N. Primerselect: Primer and probe design. Methods Mol. Biol. 70:520-527 (1997), the entire disclosures of which are hereby expressly incorporated by reference.

Further PCR-based techniques include, for example, differential display (Liang and Pardee, Science 257:967-971 (1992)); amplified fragment length polymorphism (iAFLP) (Kawamoto et al., Genome Res. 12:1305-1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888-1898 (2001)); and high coverage expression profiling (HiCEP) analysis (Fukumura et al., Nucl. Acids. Res. 31(16) e94 (2003)).

b. Microarrays

Differential gene expression can also be identified, or confirmed using the microarray technique. Thus, the expression profile of IBD-associated genes can be measured in either fresh or paraffin-embedded tissue, using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. Just as in the RT-PCR method, the source of mRNA typically is total RNA isolated from biopsy tissue or cell lines derived from cells obtained from a subject having an IBD, and corresponding normal tissues or cell lines. Thus RNA can be isolated from a variety of colonic tissues or colonic tissue-based cell lines.

In a specific embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106-149 (1996)). Microarray analysis can be performed by commercially available equipment, following manufacturer's protocols, such as by using the Affymetrix GenChip technology, or Incyte's microarray technology, or Agilent's Whole Human Genome microarray technology.

c. Serial Analysis of Gene Expression (SAGE)

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et al., Science 270:484-487 (1995); and Velculescu et al., Cell 88:243-51 (1997).

d. MassARRAY Technology

In the MassARRAY-based gene expression profiling method, developed by Sequenom, Inc. (San Diego, Calif.) following the isolation of RNA and reverse transcription, the obtained cDNA is spiked with a synthetic DNA molecule (competitor), which matches the targeted cDNA region in all positions, except a single base, and serves as an internal standard. The cDNA/competitor mixture is PCR amplified and is subjected to a post-PCR shrimp alkaline phosphatase (SAP) enzyme treatment, which results in the dephosphorylation of the remaining nucleotides. After inactivation of the alkaline phosphatase, the PCR products from the competitor and cDNA are subjected to primer extension, which generates distinct mass signals for the competitor- and cDNA-derives PCR products. After purification, these products are dispensed on a chip array, which is pre-loaded with components needed for analysis with matrix-assisted laser desorption ionization time-of-flight mass spectrometry (MALDI-TOF MS) analysis. The cDNA present in the reaction is then quantified by analyzing the ratios of the peak areas in the mass spectrum generated. For further details see, e.g. Ding and Cantor, Proc. Natl. Acad. Sci. USA 100:3059-3064 (2003).

e. Gene Expression Analysis by Massively Parallel Signature Sequencing (MPSS)

This method, described by Brenner et al., Nature Biotechnology 18:630-634 (2000), is a sequencing approach that combines non-gel-based signature sequencing with in vitro cloning of millions of templates on separate 5 μm diameter microbeads. First, a microbead library of DNA templates is constructed by in vitro cloning. This is followed by the assembly of a planar array of the template-containing microbeads in a flow cell at a high density (typically greater than 3×10⁶ microbeads/cm²). The free ends of the cloned templates on each microbead are analyzed simultaneously, using a fluorescence-based signature sequencing method that does not require DNA fragment separation. This method has been shown to simultaneously and accurately provide, in a single operation, hundreds of thousands of gene signature sequences from a yeast cDNA library.

The steps of a representative protocol for profiling gene expression using fixed, paraffin-embedded tissues as the RNA source, including mRNA isolation, purification, primer extension and amplification are given in various published journal articles (for example: Godfrey et al. J. Molec. Diagnostics 2: 84-91 (2000); Specht et al., Am. J. Pathol. 158: 419-29 (2001)). Briefly, a representative process starts with cutting about 10 microgram thick sections of paraffin-embedded tissue samples. The mRNA is then extracted, and protein and DNA are removed. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andrés et al., BioTechniques 18:42044 (1995). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MasterPure™ Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tissues can be isolated, for example, by cesium chloride density gradient centrifugation. After analysis of the RNA concentration, RNA repair and/or amplification steps may be included, if necessary, and RNA is reverse transcribed using gene specific promoters followed by PCR. Peferably, real time PCR is used, which is compatible both with quantitative competitive PCR, where internal competitor for each target sequence is used for normalization, and with quantitative comparative PCR using a normalization gene contained within the sample, or a housekeeping gene for RT-PCR. For further details see, e.g. “PCR: The Polymerase Chain Reaction”, Mullis et al., eds., 1994; and Held et al., Genome Research 6:986-994 (1996). Finally, the data are analyzed to identify the best treatment option(s) available to the patient on the basis of the characteristic gene expression pattern identified in the sample examined.

f. Immunohistochemistry

Immunohistochemistry methods are also suitable for detecting the expression levels of the IBD markers of the present invention. Thus, antibodies or antisera, preferably polyclonal antisera, and most preferably monoclonal antibodies specific for each marker are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Expression levels can also be determined at the protein level, for example, using various types of immunoassays or proteomics techniques.

In immunoassays, the target diagnostic protein marker is detected by using an antibody specifically binding to the markes. The antibody typically will be labeled with a detectable moiety. Numerous labels are available which can be generally grouped into the following categories:

Radioisotopes, such as 35S, 14C, 125I, 3H, and 131I. The antibody can be labeled with the radioisotope using the techniques described in Current Protocols in Immunology, Volumes 1 and 2, Coligen et al. (1991) Ed. Wiley-Interscience, New York, N.Y., Pubs. for example and radioactivity can be measured using scintillation counting.

Fluorescent labels such as rare earth chelates (europium chelates) or fluorescein and its derivatives, rhodamine and its derivatives, dansyl, Lissamine, phycoerythrin and Texas Red are available. The fluorescent labels can be conjugated to the antibody using the techniques disclosed in Current Protocols in Immunology, supra, for example. Fluorescence can be quantified using a fluorimeter.

Various enzyme-substrate labels are available and U.S. Pat. No. 4,275,149 provides a review of some of these. The enzyme generally catalyzes a chemical alteration of the chromogenic substrate which can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Techniques for conjugating enzymes to antibodies are described in O'Sullivan et al. (1981) Methods for the Preparation of Enzyme-Antibody Conjugates for use in Enzyme Immunoassay, in Methods in Enzym. (ed J. Langone & H. Van Vunakis), Academic press, New York 73:147-166.

Examples of enzyme-substrate combinations include, for example: horseradish peroxidase (HRPO) with hydrogen peroxidase as a substrate, wherein the hydrogen peroxidase oxidizes a dye precursor (e.g., orthophenylene diamine (OPD) or 3,3′,5,5′-tetramethyl benzidine hydrochloride (TMB)); alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate 4-methylumbelliferyl-β-D-galactosidase.

Numerous other enzyme-substrate combinations are available to those skilled in the art. For a general review of these, see U.S. Pat. Nos. 4,275,149 and 4,318,980.

Sometimes, the label is indirectly conjugated with the antibody. The skilled artisan will be aware of various techniques for achieving this. For example, the antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of the label with the antibody, the antibody is conjugated with a small hapten (e.g., digoxin) and one of the different types of labels mentioned above is conjugated with an anti-hapten antibody (e.g., anti-digoxin antibody). Thus, indirect conjugation of the label with the antibody can be achieved.

In other versions of immunoassay techniques, the antibody need not be labeled, and the presence thereof can be detected using a labeled antibody which binds to the antibody.

Thus, the diagnostic immunoassays herein may be in any assay format, including, for example, competitive binding assays, direct and indirect sandwich assays, and immunoprecipitation assays. Zola, Monoclonal Antibodies: A Manual of Techniques, pp. 147-158 (CRC Press, Inc. 1987).

Competitive binding assays rely on the ability of a labeled standard to compete with the test sample analyze for binding with a limited amount of antibody. The amount of antigen in the test sample is inversely proportional to the amount of standard that becomes bound to the antibodies. To facilitate determining the amount of standard that becomes bound, the antibodies generally are insolubilized before or after the competition, so that the standard and analyze that are bound to the antibodies may conveniently be separated from the standard and analyze which remain unbound.

Sandwich assays involve the use of two antibodies, each capable of binding to a different immunogenic portion, or epitope, of the protein to be detected. In a sandwich assay, the test sample analyze is bound by a first antibody which is immobilized on a solid support, and thereafter a second antibody binds to the analyze, thus forming an insoluble three-part complex. See, e.g., U.S. Pat. No. 4,376,110. The second antibody may itself be labeled with a detectable moiety (direct sandwich assays) or may be measured using an anti-immunoglobulin antibody that is labeled with a detectable moiety (indirect sandwich assay). For example, one type of sandwich assay is an ELISA assay, in which case the detectable moiety is an enzyme.

g. Proteomics

The term “proteome” is defined as the totality of the proteins present in a sample (e.g. tissue, organism, or cell culture) at a certain point of time. Proteomics includes, among other things, study of the global changes of protein expression in a sample (also referred to as “expression proteomics”). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the markers of the present invention.

h. 5′-multiplexed Gene Specific Priming of Reverse Transcription

RT-PCR requires reverse transcription of the test RNA population as a first step. The most commonly used primer for reverse transcription is oligo-dT, which works well when RNA is intact. However, this primer will not be effective when RNA is highly fragmented.

The present invention includes the use of gene specific primers, which are roughly 20 bases in length with a Tm optimum between about 58° C. and 60° C. These primers will also serve as the reverse primers that drive PCR DNA amplification.

An alternative approach is based on the use of random hexamers as primers for cDNA synthesis. However, we have experimentally demonstrated that the method of using a multiplicity of gene-specific primers is superior over the known approach using random hexamers.

i. Promoter Methylation Analysis

A number of methods for quantization of RNA transcripts (gene expression analysis) or their protein translation products are discussed herein. The expression level of genes may also be inferred from information regarding chromatin structure, such as for example the methylation status of gene promoters and other regulatory elements and the acetylation status of histones.

In particular, the methylation status of a promoter influences the level of expression of the gene regulated by that promoter. Aberrant methylation of particular gene promoters has been implicated in expression regulation, such as for example silencing of tumor suppressor genes. Thus, examination of the methylation status of a gene's promoter can be utilized as a surrogate for direct quantization of RNA levels.

Several approaches for measuring the methylation status of particular DNA elements have been devised, including methylation-specific PCR (Herman J. G. et al. (1996) Methylation-specific PCR: a novel PCR assay for methylation status of CpG islands. Proc. Natl. Acad. Sci. USA. 93, 9821-9826.) and bisulfite DNA sequencing (Frommer M. et al. (1992) A genomic sequencing protocol that yields a positive display of 5-methylcytosine residues in individual DNA strands. Proc. Natl. Acad. Sci. USA. 89, 1827-1831.). More recently, microarray-based technologies have been used to characterize promoter methylation status (Chen C. M. (2003) Methylation target array for rapid analysis of CpG island hypermethylation in multiple tissue genomes. Am. J. Pathol. 163, 37-45.).

j. Coexpression of Genes

A further aspect of the invention is the identification of gene expression clusters. Gene expression clusters can be identified by analysis of expression data using statistical analyses known in the art, including pairwise analysis of correlation based on Pearson correlation coefficients (Pearson K. and Lee A. (1902) Biometrika 2, 357).

k. Design of Intron-Based PCR Primers and Probes

According to one aspect of the present invention, PCR primers and probes are designed based upon intron sequences present in the gene to be amplified. Accordingly, the first step in the primer/probe design is the delineation of intron sequences within the genes. This can be done by publicly available software, such as the DNA BLAT software developed by Kent, W. J., Genome Res. 12(4):656-64 (2002), or by the BLAST software including its variations. Subsequent steps follow well established methods of PCR primer and probe design.

In order to avoid non-specific signals, it is important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Steve Rozen and Helen J. Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

The most important factors considered in PCR primer design include primer length, melting temperature (Tm), and G/C content, specificity, complementary primer sequences, and 3′-end sequence. In general, optimal PCR primers are generally 17-30 bases in length, and contain about 20-80%, such as, for example, about 50-60% G+C bases. Tm's between 50 and 80° C., e.g. about 50 to 70° C. are typically preferred.

For further guidelines for PCR primer and probe design see, e.g. Dieffenbach, C. W. et al., “General Concepts for PCR Primer Design” in: PCR Primer, A Laboratory Manual, Cold Spring Harbor Laboratory Press, New York, 1995, pp. 133-155; Innis and Gelfand, “Optimization of PCRs” in: PCR Protocols, A Guide to Methods and Applications, CRC Press, London, 1994, pp. 5-11; and Plasterer, T.N. Primerselect: Primer and probe design. Methods Mol. Biol. 70:520-527 (1997), the entire disclosures of which are hereby expressly incorporated by reference.

1. IBD Gene Set, Assayed Gene Subsequences, and Clinical Application of Gene Expression Data

An important aspect of the present invention is to use the measured expression of certain genes by colonic issue to provide diagnostic information. For this purpose it is necessary to correct for (normalize away) both differences in the amount of RNA assayed and variability in the quality of the RNA used. Therefore, the assay typically measures and incorporates the expression of certain normalizing genes, including well known housekeeping genes, such as GAPDH and Cypl. Alternatively, normalization can be based on the mean or median signal (Ct) of all of the assayed genes or a large subset thereof (global normalization approach). On a gene-by-gene basis, measured normalized amount of a patient colonic tissue mRNA is compared to the amount found in an appropriate tissue reference set. The number (N) of tissues in this reference set should be sufficiently high to ensure that different reference sets (as a whole) behave essentially the same way. If this condition is met, the identity of the individual colonic tissues present in a particular set will have no significant impact on the relative amounts of the genes assayed. Usually, the tissue reference set consists of at least about 30, preferably at least about 40 different IBD tissue specimens. Unless noted otherwise, normalized expression levels for each mRNA/tested tissue/patient will be expressed as a percentage of the expression level measured in the reference set. More specifically, the reference set of a sufficiently high number (e.g. 40) of IBD samples yields a distribution of normalized levels of each mRNA species. The level measured in a particular sample to be analyzed falls at some percentile within this range, which can be determined by methods well known in the art. Below, unless noted otherwise, reference to expression levels of a gene assume normalized expression relative to the reference set although this is not always explicitly stated.

### B.4. Antibody Compositions for Use in the Methods of the Invention

a. Anti-Ihh, Anti-DefA5 and Anti-DefA6Antibodies

The present invention further provides anti-IBD marker antibodies. Exemplary antibodies include polyclonal, monoclonal, humanized, bispecific, and heteroconjugate antibodies. As discussed herein, the antibodies may be used in the diagnostic methods for IBD, and in some cases in methods of treatment of IBD.

In one embodiment, the present invention provides the use of anti-Ihh, anti-DefA5 and/or anti-DefA6 antibodies, which may find use herein as therapeutic, diagnostic and/or prognostic agents in determining the existence, severity of and/or prognosing the disease course of an inflammatory bowel disease such as UC. Exemplary antibodies that may be used for such purposes include polyclonal, monoclonal, humanized, bispecific, and heteroconjugate antibodies. The term “antibodies” sometimes also include antigen-binding fragments. Anti-Ihh antibodies are available commercially, such as for example, from R&D Systems, Minneapolis, Minn. Anti-DefA5 and anti-DefA6 antibodies are available commercially, such as for example, from Alpha Diagnostic International, San Antonio, Tex. Alternatively, antibodies that bind specifically to Ihh, DefA5 or DefA6 as antigen may be prepared by standard methods known in the art of antibody and protein chemistry for use in the method of the invention.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp. 59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Plückthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl. Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-Ihh, anti-DefA5 and/or anti-DefA6 antibodies useful in the practice of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al, Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al, J. Mol. Biol., 196:901 (1987)). Another method uses a particular framework region derived from the consensus sequence of all human antibodies of a particular subgroup of light or heavy chains. The same framework may be used for several different humanized antibodies (Carter et al, Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-Ihh, anti-DefA5 and/or anti-DefA6 antibody antibodies are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 (1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al, J. Mol. Biol. 222:581-597 (1991), or Griffith et al, EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, while retaining similar antigen binding specificity of the corresponding full length molecule, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind separate antigens or bind to two different epitopes of a particular Ihh, DefA5 or DefA6 polypeptide described herein. Other such antibodies may combine the above Ihh, DefA5 or DefA6 binding site with a binding site for another protein. Where the bispecific antibody is useful in the diagnostic method of the invention, the second antibody arm may bind a detectable polypeptide.

Bispecific antibodies may also be used to localize agents to cells which express an IBD marker protein. These antibodies may possess an IBD marker-binding arm and an arm which binds an agent (e.g. an aminosalicylate). Bispecific antibodies can be prepared as full length antibodies or antibody fragments (e.g., F(ab′)₂ bispecific antibodies).

Methods for making bispecific antibodies are known in the art. Traditional production of full length bispecific antibodies is based on the co-expression of two immunoglobulin heavy chain-light chain pairs, where the two chains have different specificities (Millstein et al, Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies also find use in the present method of the invention by providing multiple (either different or the same) detectable markers on each antibody for improved assay detection. Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Multivalent Antibodies

A multivalent antibody may be internalized (and/or catabolized) faster than a bivalent antibody by a cell expressing an antigen to which the antibodies bind. The antibodies of the present invention (an be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

7. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al, Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

8. Immunoconjugates

The invention also pertains to immunoconjugates comprising an antibody conjugated to a cytotoxic agent such as a chemotherapeutic agent, a growth inhibitory agent, a toxin (e.g., an enzymatically active toxin of bacterial, fungal, plant, or animal origin, or fragments thereof), or a radioactive isotope (i.e., a radioconjugate) and/or a detectable label.

a. Chemotherapeutic agents

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

### B.5. Ihh, DefA5 or DefA6 Binding Oligopeptides

Ihh, DefA5 or DefA6 binding oligopeptides of the present invention are oligopeptides that bind, preferably specifically, to a Ihh, DefA5 or DefA6 polypeptide as described herein. Ihh, DefA5 or DefA6 binding oligopeptides may be chemically synthesized using known oligopeptide synthesis methodology or may be prepared and purified using recombinant technology. Ihh, DefA5 or DefA6-binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a Ihh, DefA5 or DefA6-polypeptide as described herein. Ihh, DefA5 and/or DefA6 binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al, Cancer Research, 58(15): 3209-3214 (1998); Jiang et al., Infection & Immunity, 65(11): 4770-4777 (1997); Ren et al., Gene, 195(2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol. Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

### B.6. Polypeptide Variants

In addition to the polypeptides, antibodies and Ihh, DefA5 or DefA6 binding polypeptides described herein, it is contemplated that variants of such molecules can be prepared for use with the invention herein. Such variants can be prepared by introducing appropriate nucleotide changes into the encoding DNA, and/or by synthesis of the desired antibody or polypeptide. Those skilled in the art will appreciate that amino acid changes may alter post-translational processes of these molecules, such as changing the number or position of glycosylation sites or altering the membrane anchoring characteristics.

Variations in amino acid sequence can be made, for example, using any of the techniques and guidelines for conservative and non-conservative mutations set forth, for instance, in U.S. Pat. No. 5,364,934. Variations may be a substitution, deletion or insertion of one or more codons encoding the amino acid sequence that results in a change in the amino acid sequence as compared with the native sequence. Optionally the variation is by substitution of at least one amino acid with any other amino acid in one or more of the domains of the amino acid sequence of interest. Guidance in determining which amino acid residue may be inserted, substituted or deleted without adversely affecting the desired activity may be found by comparing the sequence of the amino acid sequence of interest with homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Fragments of the various polypeptides are provided herein. Such fragments may be truncated at the N-terminus or C-terminus, or may lack internal residues, for example, when compared with a full length native antibody or protein. Such fragments which lack amino acid residues that are not essential for a desired biological activity are also useful with the disclosed methods.

The above polypeptide fragments may be prepared by any of a number of conventional techniques. Desired peptide fragments may be chemically synthesized. An alternative approach involves generating such fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding the desired fragment by polymerase chain reaction (PCR). Oligonucleotides that define the desired termini of the DNA fragment are employed at the 5′ and 3′ primers in the PCR. Preferably, such fragments share at least one biological and/or immunological activity with the corresponding full length molecule.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened in order to identify the desired variant.

TABLE 6

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser, Ala | Ser |
| Gln (Q) | Asn; Glu | Asn |
| Glu (E) | Asp, Gln | Asp |
| Gly (G) | Pro; Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Leu |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in function or immunological identity of the Ihh, DefA5 or DefA6 polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: Norleucine, Met, Ala, Val, Leu, Ile;

(2) neutral hydrophilic: Cys, Ser, Thr; Asn; Gln

(3) acidic: Asp, Glu;

(4) basic: H is, Lys, Arg;

(5) residues that influence chain orientation: Gly, Pro; and

(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the anti-Ihh, DefA5 or DefA6 molecule.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the Ihh, DefA5 or DefA6 polypeptide also may be substituted, generally with serine, to improve the oxidative stability of the molecule and prevent aberrant crosslinking. Conversely, cysteine bond(s) may be added to such a molecule to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of Ihh, DefA5 or DefA6 polypeptides are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of a native sequence or an earlier prepared variant.

### B.7. Modifications of Polypeptides

Polypeptides and/or antibodies that have been covalently modified may also be suitable for use within the scope of this invention. One type of covalent modification includes reacting targeted amino acid residues of such antibodies and polypeptides with an organic derivatizing agent that is capable of reacting with selected side chains or the N- or C-terminal residues of such antibodies and polypeptides. Derivatization with bifunctional agents is useful, for instance, for crosslinking the preceding molecules to a water-insoluble support matrix or surface for use in purification. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of the polypeptides or antibodies comprises altering the native glycosylation pattern of the antibody or polypeptide. “Altering the native glycosylation pattern” is intended for purposes herein to mean deleting one or more carbohydrate moieties found in native sequence (either by removing the underlying glycosylation site or by deleting the glycosylation by chemical and/or enzymatic means), and/or adding one or more glycosylation sites that are not present in the respective native sequence. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites may be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original such antibody or polypeptide (for O-linked glycosylation sites). Such antibody or polypeptide sequence may optionally be altered through changes at the DNA level, particularly by mutating the DNA encoding the preceding amino acid sequences at preselected bases such that codons are generated that will translate into the desired amino acids.

Another means of increasing the number of carbohydrate moieties is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al, Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification comprises linking to one of a variety of nonproteinaceous polymers, e.g., polyethylene glycol (PEG), polypropylene glycol, or polyoxyalkylenes, in the manner set forth in U.S. Pat. No. 4,640,835; 4,496,689; 4,301,144; 4,670,417; 4,791,192 or 4,179,337. The Ihh, DefA5 or DefA6 polypeptide also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Modifications forming chimeric molecules results from fusions of one polypeptide to another, heterologous polypeptide or amino acid sequence are contemplated for use with the present methods.

In one embodiment, such a chimeric molecule comprises a fusion of a polypeptide with a tag polypeptide which provides an epitope to which an anti-tag antibody can selectively bind. The epitope tag is generally placed at the amino- or carboxyl-terminus of such antibody or polypeptide. The presence of such epitope-tagged forms of such antibodies or polypeptides can be detected using an antibody against the tag polypeptide. Also, provision of the epitope tag enables such antibodies or polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of a polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a preceding antibody or polypeptide in the place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also U.S. Pat. No. 5,428,130 issued Jun. 27, 1995.

### B.8. Preparation of Polypeptides

The description below relates primarily to production of polypeptides by culturing cells transformed or transfected with a vector containing nucleic acid such antibodies, polypeptides and oligopeptides. The term “polypeptides” may include antibodies, polypeptides and oligopeptides. It is, of course, contemplated that alternative methods, which are well known in the art, may be employed to prepare such antibodies, polypeptides and oligopeptides. For instance, the appropriate amino acid sequence, or portions thereof, may be produced by direct peptide synthesis using solid-phase techniques [see, e.g., Stewart et al., Solid-Phase Peptide Synthesis, W.H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of such antibodies, polypeptides or oligopeptides may be chemically synthesized separately and combined using chemical or enzymatic methods to produce the desired product.

1. Isolation of DNA Encoding a Polypeptide

DNA encoding a polypeptide may be obtained from a cDNA library prepared from tissue believed to possess such antibody, polypeptide or oligopeptide mRNA and to express it at a detectable level. Accordingly, DNA encoding such polypeptides can be conveniently obtained from a cDNA library prepared from human tissue, a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). Alternatively, PCR methodology may be used. [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for Ihh, DefA5 or DefA6 polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansouret al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed in suitable cells (e.g. CHO cells).

In addition to prokaryotes, eukaryotic microbes such as filamentous fungi or yeast are suitable cloning or expression hosts for vectors encoding desired polypeptides. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742

), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al, Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida, Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs, 269 (1982).

Suitable host cells for the expression of glycosylated polypeptide production are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al, Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for desired polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding the respective Ihh, DefA5 or DefA6 polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the ordinarily skilled artisan.

The desired polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA encoding the mature sequence that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 211 plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up nucleic acid encoding the desire protein, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7[Stinchcomb et al, Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the nucleic acid sequence encoding the desired amino acid sequence, in order to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgamo (S.D.) sequence operably linked to the DNA encoding the desired protein sequence.

Examples of suitable promoting sequences for use with yeast hosts include the promoters for 3-phosphoglycerate kinase [Hitzeman et al, J. Biol. Chem., 255:2073 (1980)] or other glycolytic enzymes [Hess et al., J. Adv. Enzyme Reg., 7:149 (1968); Holland, Biochemist, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

DNA Transcription in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the desired polypeptide may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the coding sequence of the preceding amino acid sequences, but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the respective antibody, polypeptide or oligopeptide described in this section.

Still other methods, vectors, and host cells suitable for adaptation to the synthesis of the respective antibody, polypeptide or oligopeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the Ihh, DefA5 or DefA6 polypeptide may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCINJ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture, feces or body fluids, to quantitate directly the expression of gene product. Antibodies useful for immunohistochemical staining and/or assay of sample fluids may be either monoclonal or polyclonal, and may be prepared in any mammal. Conveniently, the antibodies suitable for the present method may be prepared against a native sequence polypeptide or oligopeptide, or against exogenous sequence fused to DNA and encoding a specific antibody epitope of such a polypeptide or oligopeptide.

6. Protein Purification

Polypeptides may be recovered from culture medium or from host cell lysates. If membrane-bound, it can be released from the membrane using a suitable detergent solution (e.g. Triton-X 100) or by enzymatic cleavage. Cells employed in expression of the preceding can be disrupted by various physical or chemical means, such as freeze-thaw cycling, sonication, mechanical disruption, or cell lysing agents.

It may be desirable to purify the preceding from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the desired molecules. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular antibody, polypeptide or oligopeptide produced for the claimed methods.

When using recombinant techniques, the Ihh, DefA5 or DefA6 polypeptide can be produced intracellularly, in the periplasmic space, or directly secreted into the medium. If such molecules are produced intracellularly, as a first step, the particulate debris, either host cells or lysed fragments, are removed, for example, by centrifugation or ultrafiltration. Carter et al, Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Purification can occur using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABXJresin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSEJ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### B.9 Kits of the Invention

The materials for use in the methods of the present invention are suited for preparation of kits produced in accordance with well known procedures. The invention thus provides kits comprising agents, which may include gene-specific or gene-selective probes and/or primers, for quantitating the expression of the disclosed genes for IBD. Such kits may optionally contain reagents for the extraction of RNA from samples, in particular fixed paraffin-embedded tissue samples and/or reagents for RNA amplification. In addition, the kits may optionally comprise the reagent(s) with an identifying description or label or instructions relating to their use in the methods of the present invention. The kits may comprise containers (including microtiter plates suitable for use in an automated implementation of the method), each with one or more of the various reagents (typically in concentrated form) utilized in the methods, including, for example, pre-fabricated microarrays, buffers, the appropriate nucleotide triphosphates (e.g., dATP, dCTP, dGTP and dTTP; or rATP, RCTP, rGTP and UTP), reverse transcriptase, DNA polymerase, RNA polymerase, and one or more probes and primers of the present invention (e.g., appropriate length poly(T) or random primers linked to a promoter reactive with the RNA polymerase).

### B.10 Reports of the Invention

The methods of this invention, when practiced for commercial diagnostic purposes generally produce a report or summary of the normalized expression levels of one or more of the selected genes. The methods of this invention will produce a report comprising a prediction of the clinical outcome of a subject diagnosed with an IBD before and after any surgical procedure to treat the IBD. The methods and reports of this invention can further include storing the report in a database. Alternatively, the method can further create a record in a database for the subject and populate the record with data. In one embodiment the report is a paper report, in another embodiment the report is an auditory report, in another embodiment the report is an electronic record. It is contemplated that the report is provided to a physician and/or the patient. The receiving of the report can further include establishing a network connection to a server computer that includes the data and report and requesting the data and report from the server computer.

The methods provided by the present invention may also be automated in whole or in part.

All aspects of the present invention may also be practiced such that a limited number of additional genes that are co-expressed with the disclosed genes, for example as evidenced by high Pearson correlation coefficients, are included in a prognostic or predictive test in addition to and/or in place of disclosed genes.

### B.11. Pharmaceutical Formulations

Therapeutic formulations IBD therapeutic agent (“therapeutic agent”) used in accordance with the present invention may be prepared for storage by mixing the therapeutic agent(s) having the desired degree of purity with optional pharmaceutically acceptable carriers, excipients or stabilizers (Remington: The Science of Practice of Pharmacy, 20th edition, Gennaro, A. et al., Ed., Philadelphia College of Pharmacy and Science (2000)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWEEN₇, PLURONICS₇ or polyethylene glycol (PEG). The antibody preferably comprises the antibody at a concentration of between 5-200 mg/ml, preferably between 10-100 mg/ml.

The formulations herein may also contain more than one active compound as necessary for the particular indication being treated, preferably those with complementary activities that do not adversely affect each other. For example, in addition to the preceding therapeutic agent(s), it may be desirable to include in the formulation, an additional antibody, e.g., a second such therapeutic agent, or an antibody to some other target such as a growth factor that affects the growth of the glioma. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy, supra.

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT₇ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### B.12 Articles of Manufacture and Kits

For therapeutic applications, the article of manufacture comprises a container and a label or package insert on or associated with the container indicating a use for the detection and quantitation of Ihh expression in a gastrointestinal tissue sample or cells from a mammal. In an embodiment, the container, label or package insert indicates that the gastrointestinal tissue or cells are from colon or sigmoid colon of a mammal. In an embodiment, the container, label or package insert indicates that decreased Ihh expression relative to a control sample is indicative of IBD, including without limitation UC, in the mammal. Suitable containers include, for example, bottles, vials, syringes, etc. The containers may be formed from a variety of materials such as glass or plastic. Additionally, the article of manufacture may further comprise a second container comprising a buffer or other reagent (such as detectable label) useful for carrying out the detection. It may further include other materials desirable from a commercial and user standpoint, including other buffers, diluents, filters, and dyes.

For isolation and purification of Ihh, DefA5 or DefA6 polypeptide, the kit can contain the respective Ihh-, DefA5, and/or DefA6-binding reagent coupled to beads (e.g., sepharose beads). Kits can be provided which contain such molecules for detection and quantitation of Ihh, DefA5 or DefA6 polypeptide in vitro, e.g., in an ELISA or a Western blot. As with the article of manufacture, the kit comprises a container and a label or package insert on or associated with the container. The container holds a composition comprising at least one such Ihh-, DefA5, and/or DefA6-binding antibody, oligopeptide or organic molecule useable with the invention. Additional containers may be included that contain, e.g., diluents and buffers, control antibodies. The label or package insert may provide a description of the composition as well as instructions for the intended in vitro or diagnostic use.

### B.13. Sense and Anti-Sense Ihh-, DefA5 and DefA6-Encoding Nucleic Acids

Molecules that would be expected to bind to nucleic acids encoding the Ihh (SEQ ID NO:2), DefA5 (SEQ ID NO:4) or DefA6 (SEQ ID NO:6) polypeptides include sense and antisense oligonucleotides, which comprise a single-stranded nucleic acid sequence (either RNA or DNA) capable of binding to target Ihh, DefA5 or DefA6 mRNA (sense) or Ihh, DefA5 or DefA6 DNA (antisense) sequences. Antisense or sense oligonucleotides, according to the present invention, comprise a fragment of the coding region of the respective Ihh, DefA5 or DefA6 DNA or its complement. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988). The sense and/or antisense oligonucleotides hybridizable to an Ihh, DefA5 or DefA6 gene, respectively, are useful, for example, for detecting the presence of Ihh, DefA5 or DefA6 DNA or mRNA in a tissue or cell sample gastrointestinal tissue or cells of mammal according to the invention. The sense and/or antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Sense and antisense oligonucleotides include without limitation primers and probes useful in PCR, RT-PCR, hybridization methods, in-situ hybridization, and the like.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

All publications mentioned herein are incorporated herein by reference to disclose and describe the methods and/or materials in connection with which the publications are cited. Publications cited herein are cited for their disclosure prior to the filing date of the present application. Nothing here is to be construed as an admission that the inventors are not entitled to antedate the publications by virtue of an earlier priority date or prior date of invention. Further the actual publication dates may be different from those shown and require independent verification.

The following nonlimiting examples are provided for illustrative purposes and are not intended to limit the scope of the invention. Commercially available reagents referred to in the examples were used according to manufacturer's instructions unless otherwise indicated. The source of those cell lines identified in the following examples, and/or throughout the specification, by ATCC accession numbers is the American Type Culture Collection, Manassas, Va.

### EXAMPLES

### Example 1

### RT-PCR and Histologic Analysis to Detect Downregulation of Ihh Gene Expression in Gastrointestinal tissue

To determine whether the expression pattern of Ihh was altered in IBD, surgical resection specimens and endoscopic biopsies from patients with UC and CD were obtained. Mucosal biopsies from specified anatomical locations within the gastrointestinal track were flash frozen in liquid nitrogen and stored at −80° C. For histological analysis, inflammation status was scored for each biopsy sample using standard pathological criteria. Patients were diagnosed with ulcerative colitis based on the criteria described by Lennard-Jones (Lenard-Jones, J. E., Scand J. Gastroenterol. Suppl. 170:2-6 (1989)). Patients symptoms were evaluated using the clinical colitis activity index (SCCAI) (Walmsley, R. S. et al., Gut 43:29-32 (1998)). Each endoscopic biopsy was categorized by patient status, biopsy inflammation status, and anatomical location. Inflammation scoring was based on inflammatory cell type predominance: neutrophil predominance=acute inflammation; neutrophils and mononuclear inflammatory cells=chronic active; and predominantly mononuclear inflammatory cells=chronic. Inflammatory status was graded on paired biopsies for histology as inflamed (chronic or acute inflammation) or non-inflamed.

RT-PCR analysis of Ihh expression. To quantify mRNA levels of Ihh, RT-PCR was used. Diseased tissue specimens were analyzed which included acute or chronic inflammation and normal histology, potentially allowing analysis in IBD independent of the inflammatory signal. Real-time RT-PCR was subsequently performed for Ihh expression in 10 acutely inflamed UC specimens and 10 non-inflamed healthy control specimen, all sampled from the sigmoid colon. One RNA amplification cycle was carried out using the MessageAmp™ II aRNA Amplification Kit protocol (Ambion technologies, Austin, Tex.). Reverse transcription PCR was then performed on 50 ng of RNA using Stratagene model MX4000 (La Jolla, Calif., USA). TaqMan™ PCR system (Applied Biosystems) primers and probes were prepared by standard techniques. The sequences for the Ihh forward primer, reverse primer and TaqMan hybridization probe were as follows: forward—cttcagcgatgtgctcattt (SEQ ID NO:7); reverse—ctgagtctcgatgacctgga (SEQ ID NO:8); Hybridization oligo tactggaccgcgagccccac (SEQ ID NO:9). PCR conditions were 48° C. for 30 minutes, 95° C. hold for 10 minutes, followed by 40 cycles of 30 second 95° C. melt and 1 minute 60° C. anneal/extend. Quantitation of product was performed by normalizing to RPL19. Results were analyzed using SAS and JMP software (SAS, North Carolina).

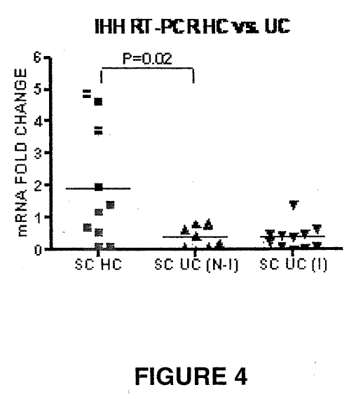

Using RT-PCR analysis, Ihh mRNA was shown to be decreased in UC specimens irrespective of inflammatory status (p=0.02) (FIG. 4). Thus, a diagnosis of UC may be made, independent of inflammation status, based on a showing of Ihh downregulation in colon tissue, including sigmoid colon tissue. In addition, following initial testing for Ihh expression and therapeutic treatment for IBD (including UC), subsequent detection of Ihh expression in a gastrointestinal tissue or cells sample (such as without limitation a sigmoid colon sample) is useful for determine response or lack of response to the therapeutic.

Microarray analysis of Ihh expression: Total RNA is extracted from each biopsy using the RNeasy™ Kit (Qiagen, Valencia, Calif.) according to manufacturer's instructions. To evaluate RNA purity and integrity, 1 μL of total RNA is assessed for each sample with the Agilent 2100 Bioanalyzer™ using the Pico LabChip™ reagent set (Agilent Technologies, Palo Alto, Calif.).

Microarray analysis is performed as follows. It is noted that other products and protocols may be used for performing the studies disclosed herein. Briefly, a 1 μg aliquot of total RNA is amplified using the low RNA input fluorescent linear amplification protocol (Agilent Technologies, Palo Alto, Calif.). A T7 RNA polymerase single round of linear amplification is carried out to incorporate cyanine-3- or cyanine 5-labeled CTP labeled cRNA targets for oligonucleotide array. The amplified cRNA is then purified using the RNeasy Mini Kit™ protocol (Qiagen) and 1 μL of amplified cRNA is quantified using the NanoDrop ND-1000™Spectrophotometer (NanoDrop Technologies, Wilmington, Del.). A 750 ng sample of Agilent universal human reference labeled with Cy-3 and 750 ng of the test sample labeled with Cy-5 ae fragmented to 30 minutes at 60 C before loading the samples onto a microarray chip comprising an Ihh gene nucleic acid sequence. The samples are hybridized for 18 hours at 60° C. with constant rotation. Slides are washed and dried using the Agilent stabilization and drying solution protocol (Agilent Technologies). Microarray slides are scanned using the Agilent G2505B™ microarray scanner (Agilent Technologies). Expression signals are calculated using the Agilent feature extraction softward (version 7.5, Agilent Technologies). The distribution of log intensities for each sample are plotted and outlying samples (greater than 2 standard deviations from the mean) are excluded from analysis.

### Example 2

### Microarray and Histologic Analysis to Detect Upregulation of DefA5 Gene Expression in Gastrointestinal Tissue

Increased DefA5 expression at the RNA level was detected in ulcerative colitis patients using Taqman® PCR analysis (using standard techniques) on biopsy lysates.

Real time polymerase chain reaction (RT-PCR) analysis was performed as follows. Briefly, one RNA amplification cycle was carried out using the MessageAmp™ II aRNA Amplification Kit protocol (Ambion Technologies, Austin, Tex.). Reverse transcriptase PCR was then performed on 50 ng of RNA using Stratgene model MX4000™ Multiplex Quantitative PCR system (Stratagene, La Jolla, Calif.). TaqMan™ PCR system (Applied Biosystems) primers and probes were prepared by standard techniques. The sequences for the DefA5 forward primer, reverse primer and TaqMan hybridization probe were as follows: forward—gctacccgtgagtccctct (SEQ ID NO:10); reverse—tcttgcactgctttggtttc (SEQ ID NO:11); hybridization probe—tgtgtgaaatcagtggccgcct (SEQ ID NO:12). PCR conditions were 48° C. for 30 minutes, 95° C. hold for 10 minutes, followed by 40 cycles of 30 second 95° C. melt and 1 minute 60° C. anneal/extend. Absolute quantification of product was calculated by normalizing to RPL19. Results were analyzed using SAS and JMP software (SAS, North Carolina). Microarray data were analyzed using the Rosetta Resolver™ software (Rosetta BioSoftware, Seattle, Wash.). Statistical significance of the microarray data was determined by Student's unpaired t test. A p value <0.01 and a fold change of greater or less than 1.5 were considered statistically significant. Gene ontology was analyzed using Ingenuity™ software (Ingenuity Systems, Mountain View, Calif.). The Mann-Whitney U test was used to analyze the real time PCR data. A p value <0.05 was considered significant.

The relative increase (+) or decrease (−) in DefA5 expression in various UC tissue biopsies using RT-PCR are shown in Table 7. p values are shown below the relative gene expression value.

TABLE 7

| | | Non-inflamed | Inflamed UC | Inflamed UC |
|---|---|---|---|---|
| | | UC Sigmoid v. | Sigmoid v. | Sigmoid |
| | | Non-inflamed | Inflamed | v.Non- |
| | All UC v. | Control Sigmoid | Conrol | Inflamed UC |
| Gene | Controls | Colon | Sigmoid Colon | Sigmoid Colon |
| DefA5 | +3.25 | +1.02 | +7.27 | +8.44 |
| | (0.00003) | (0.89) | (6.3 × 10\hat{ }−30) | (<10\hat{ }−45) |

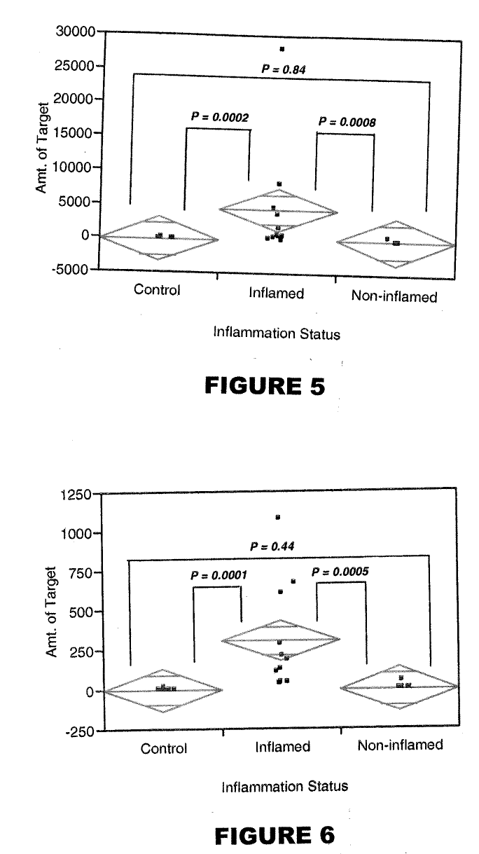

The results in Table 7 indicate that DefA5 expression is upregulated in inflamed UC tissue of the sigmoid colon (See also FIG. 5). DefA5 expression was observed in the terminal ileum of control and UC patients. In control patients, levels of DefA5 expression decreased with increasing distance of the biopsy from the terminal ileum. By contrast, in acute and chronically inflamed UC biopsies, an increase in DefA5 expression was observed throughout the ascending, descending and sigmoid colon.

RNA isolation and microarray analysis: The biopsies weighed between 0.2 mg and 16.5 mg with a mean weight of 5.5 mg. Total RNA was extracted from each biopsy using the micro total RNA isolation from animal tissues protocol (RNeasy™ Kit, Qiagen, Valencia, Calif.) according to manufacturer's instructions. To evaluate RNA purity and integrity, 1 μL of total RNA was assessed for each sample with the Agilent 2100 Bioanalyzer™ using the Pico LabChip™ reagent set (Agilent Technologies, Palo Alto, Calif.).

Histological analysis performed as follows: Inflammation status is scored for each biopsy sample using standard pathological criteria. Patients are diagnosed with ulcerative colitis based on criteria according to Lennard-Jones (Lenard-Jones, J. E., Scand J. Gastroenterol. Suppl. 170:2-6 (1989)). Patients symptoms were evaluated using the clinical colitis activity index (SCCAI) (Walmsley, R. S. et al., Gut 43:29-32 (1998)). Each endoscopic biopsy was categorized by patient status, biopsy inflammation status, and anatomical location. Inflammation scoring was based on inflammatory cell type predominance: neutrophil predominance=acute inflammation; neutrophils and mononuclear inflammatory cells=chronic active; predominantly mononuclear inflammatory cells=chronic.

In summary, DefA5 expression in ulcerative colitis correlated with the local inflammation status observed in the biopsy.

### Example 3

### Microarray and Histologic Analysis to Detect Upregulation of DefA6 Gene Expression in Gastrointestinal Tissue

Defensin alpha 6 is normally expressed by Paneth cells in the small intestine crypt epithelium and not in colon epithelial cells. Increased DefA6 expression at the RNA level was detected in ulcerative colitis using Agilent microarray analysis and in Taqman® PCR analysis (using standard techniques) on biopsy lysates. Histologic staining was also performed to determine whether increased DefA6 protein expression could be seen in formalin fixed colon biopsies.

RNA isolation and microarray analysis: The biopsies weighed between 0.2 mg and 16.5 mg with a mean weight of 5.5 mg. Total RNA was extracted from each biopsy using the micro total RNA isolation from animal tissues protocol (RNeasy™ Kit, Qiagen, Valencia, Calif.) according to manufacturer's instructions. To evaluate RNA purity and integrity, 1 μL of total RNA was assessed for each sample with the Agilent 2100 Bioanalyzer™ using the Pico LabChip™ reagent set (Agilent Technologies, Palo Alto, Calif.).

Microarray analysis was performed as follows. Briefly, a 1 μg aliquot of total RNA was amplified using the low RNA input fluorescent linear amplification protocol (Agilent Technologies, Palo Alto, Calif.). A T7 RNA polymerase single round of linear amplification was carried out to incorporate cyanine-3- or cyanine 5-labeled CTP labeled cRNA targets for oligonucleotide array. The amplified cRNA was then purified using the RNeasy Mini Kit™ protocol (Qiagen) and 1 μL of amplified cRNA was quantified using the NanoDrop ND-1000™ Spectrophotometer (NanoDrop Technologies, Wilmington, Del.). A 750 ng sample of Agilent universal human reference labeled with Cy-3 and 750 ng of the test sample labeled with Cy-5 were fragmented to 30 minutes at 60° C. before loading the samples onto Agilent whole human genome oligo microarray chips G4112A (Agilent Technologies, Palo Alto, Calif.). The samples were hybridized for 18 hours at 60° C. with constant rotation. Slides were washed and dried using the Agilent stabilization and drying solution protocol (Agilent Technologies). Microarray slides were scanned using the Agilent G2505B™ microarray scanner (Agilent Technologies). The samples were hybridized for 18 hours at 60° C. with constant rotation. Slides were washed and dried using the Agilent stabilization and drying solution protocol (Agilent Technologies). Microarray slides were scanned using the Agilent G2505B microarray scanner. Expression signals were calculated using the Agilent feature extraction softward (version 7.5, Agilent Technologies). The distribution of log intensities for each sample were plotted and outlying samples (greater than 2 standard deviations from the mean) were excluded from analysis.

Real time polymerase chain reaction (RT-PCR) analysis was performed as follows. Briefly, one RNA amplification cycle was carried out using the MessageAmp™II aRNA Amplification Kit protocol (Ambion Technologies, Austin, Tex.). Reverse transcriptase PCR was then performed on 50 ng of RNA using Stratgene model MX4000™ Multiplex Quantitative PCR system (Stratagene, La Jolla, Calif.). TaqMan™ PCR system (Applied Biosystems) primers and probes were prepared by standard techniques. The sequences for the DefA6 forward probe, reverse probe and TaqMan probe were as follows: forward—agagctttgggctcaacaag (SEQ ID NO:13); reverse—atgacagtgcaggtcccata (SEQ ID NO: 14); hybridization probe—cacttgccattgcagaaggtcctg (SEQ ID NO: 15). PCR conditions were 48° C. for 30 minutes, 95° C. hold for 10 minutes, followed by 40 cycles of 30 second 95° C. melt and 1 minute 60° C. anneal/extend. Absolute quantification of product was calculated by normalizing to RPL19. Results were analyzed using SAS and JMP software (SAS, North Carolina). Microarray data were analyzed using the Rosetta Resolver™ software (Rosetta BioSoftware, Seattle, Wash.). Statistical significance of the microarray data was determined by Student's unpaired t test. A p value <0.01 and a fold change of greater or less than 1.5 were considered statistically significant. Gene ontology was analyzed using Ingenuity™ software (Ingenuity Systems, Mountain View, Calif.). The Mann-Whitney U test was used to analyze the real time PCR data. A p value <0.05 was considered significant.

The relative increase (+) or decrease (−) in DefA6 expression in various UC tissue are shown in Table 8. p valueshown in parentheses below the relative gene expression value.

TABLE 8

| | | Non-inflamed UC | Inflamed UC | Inflamed UC |
|---|---|---|---|---|
| | | Sigmoid v. Non- | Sigmoid v. Inflamed | Sigmoid v. Non- |
| | | inflamed Control | Conrol Sigmoid | Inflamed UC |
| Gene | All UC v. Controls | Sigmoid Colon | Colon | Sigmoid Colon |
| DefA6 | +2.18 | −1.09 | +4.41 | +6.72 |
| | (6.95 × 10\hat{ }−7) | (0.34) | (9.7 × 10\hat{ }−9) | (4.16 × 10\hat{ }−19) |

The results in Table 8 indicate that DefA6 expression is upregulated in inflamed UC tissue of the sigmoid colon (See also FIG. 6). DefA6 expression was observed in the terminal ileum of control and UC patients. In control patients, levels of DefA expression decreased with increasing distance of the biopsy from the terminal ileum. By contrast, in acute and chronically inflamed UC biopsies, an increase in DefA6 expression was observed throughout the ascending, descending and sigmoid colon.

Histological analysis: Inflammation status was scored for each biopsy sample using standard pathological criteria. Patients were diagnosed with ulcerative colitis based on the criteria of Lennard-Jones (Lenard-Jones, J. E., Scand J. Gastroenterol. Suppl. 170:2-6 (1989)). Patients symptoms were evaluated using the clinical colitis activity index (SCCAI) (Walmsley, R. S. et al., Gut 43:29-32 (1998)). Each endoscopic biopsy was categorized by patient status, biopsy inflammation status, and anatomical location. Inflammation scoring was based on inflammatory cell type predominance: neutrophil predominance=acute inflammation; neutrophils and mononuclear inflammatory cells=chronic active; predominantly mononuclear inflammatory cells=chronic.

There was no DefA6 staining in colon biopsies from non-IBD control patients with no histologic evidence of inflammation. The biopsy from one non-IBD control patient with a diagnosis of microscopic colitis was evaluated. In that patient, DefA6 was present in sigmoid colon crypt epithelial cells.

In ulcerative colitis patients, 21 patients had scattered or clustered DefA6 staining in crypt epithelial cells of the sigmoid colon, descending colon, transverse colon, or rectum. Twenty of 21 positive patients had histologic evidence of chronic or chronic-active inflammation in their biopsy tissue. The remaining patient had predominantly acute (neutrophilic) inflammation. No patients with positive DefA6 staining in the colon had uninflamed biopsies.

There were 18 ulcerative colitis patients with no evidence of DefA6 staining in colon epithelium. The majority of these patients (10) had no histologic evidence of inflammation in the biopsy tissue. Six of the remaining patients had predominantly neutrophilic inflammation (acute inflammation) and two had chronic/chronic-active inflammation.

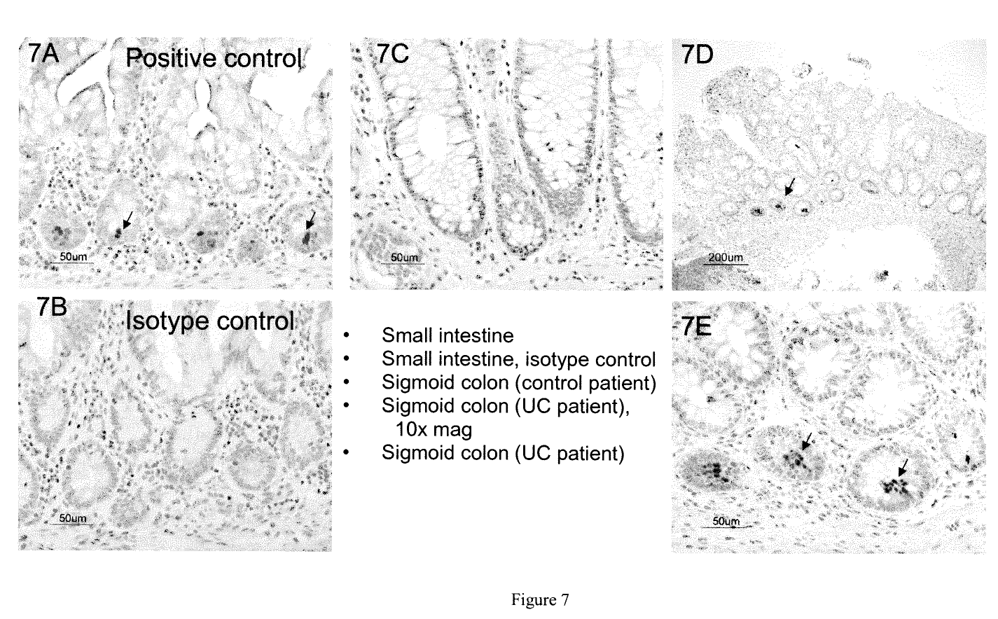

FIGS. 7A-7E are photographs of control small intestine tissue and sigmoid colon tissue as well as test sigmoid colon tissue from a UC patient stained for the presence DefA6 in tissue. Rabbit anti-human DefA6 (Alpha Diagnostic International, San Antonio, Tex.) followed by biotinylated goat ant-rabbit and peroxidase detection. The photographs of FIGS. 7A-C and 7E are 40× magnification, while FIG. 7D is 10× magnification. Arrows in FIGS. 7A, 7D, and 7E indicate positive staining of DefA6 in crypt epithelial cells.

In summary, DefA6 expression in ulcerative colitis correlated with the local inflammation status observed in the biopsy. None of the uninflamed biopsies had DefA6 staining. In addition, patients with chronic or chronic-active inflammation were more likely to have positive DefA6 staining than patients with acute inflammation.

### Example 4

### In Situ Hybridization

In situ hybridization is a powerful and versatile technique for the detection and localization of nucleic acid sequences within cell or tissue preparations. It may be useful, for example, to identify sites of gene expression, analyze tissue distribution of transcription, and follow changes in specific mRNA synthesis of Ihh, DefA5 and/or DefA6.

In situ hybridization is performed following an optimized version of the protocol by Lu and Gillett, Cell Vision 1:169-176 (1994), using PCR-generated ³³P-labeled riboprobes. Briefly, formalin-fixed, paraffin-embedded human tissues are sectioned, deparaffinized, deproteinated in proteinase K (20 g/ml) for 15 minutes at 37 EC, and further processed for in situ hybridization as described by Lu and Gillett, supra. A [³³-P] UTP-labeled antisense riboprobe are generated from a PCR product and hybridized at 55 EC overnight. Useful probes comprising a portion of the sequence of the gene of interest, or its complement depending upon whether sense or antisense sequences are to be detected, where the sequence is of sufficient length to specifically hybridize with the gene of interest, it's transcript or fragments thereof. The slides are dipped in Kodak NTB2 nuclear track emulsion and exposed for 4 weeks.

³³P-Riboprobe Synthesis

6.0 μl (125 mCi) of ³³P-UTP (Amersham BF 1002, SA<2000 Ci/mmol) were speed vac dried. To each tube containing dried ³³P-UTP, the following ingredients were added:

2.0 μl 5× transcription buffer

1.0 μl DTT (100 mM)

2.0 μl NTP mix (2.5 mM: 10μ; each of 10 mM GTP, CTP & ATP+10 μl H₂O)

1.0 μl UTP (50 μM)

1.0 μl Rnasin

1.0 μl DNA template (1 μg)

1.0 μl H₂O

1.0 μl RNA polymerase (for PCR products T3=AS, T7=S usually)

The tubes are incubated at 37 EC for one hour. 1.0 μl RQ1 DNase is added, followed by incubation at 37 EC for 15 minutes. 90 μl TE (10 mM Tris pH 7.6/1 mM EDTA pH 8.0) are added, and the mixture was pipetted onto DE81 paper. The remaining solution is loaded in a Microcon-50 ultrafiltration unit, and spun using program 10 (6 minutes). The filtration unit is inverted over a second tube and spun using program 2 (3 minutes). After the final recovery spin, 100 μl TE is added. 1 μl of the final product is pipetted on DE81 paper and counted in 6 ml of Biofluor II.

The probe is run on a TBE/urea gel. 1-3 μl of the probe or 5 μl of RNA Mrk III is added to 3 μl of loading buffer. After heating on a 95 EC heat block for three minutes, the probe is immediately placed on ice. The wells of gel are flushed, the sample loaded, and run at 180-250 volts for 45 minutes. The gel is wrapped in saran wrap and exposed to XAR film with an intensifying screen in −70 EC freezer one hour to overnight.

³³P-Hybridization

A. Pretreatment of Frozen Sections

The slides are removed from the freezer, placed on aluminium trays and thawed at room temperature for 5 minutes. The trays are placed in 55 EC incubator for five minutes to reduce condensation. The slides are fixed for 10 minutes in 4% paraformaldehyde on ice in the fume hood, and washed in 0.5×SSC for 5 minutes, at room temperature (25 ml 20×SSC+975 ml SQ H₂O). After deproteination in 0.5 μg/ml proteinase K for 10 minutes at 37 EC (12.5 μl of 10 mg/ml stock in 250 ml prewarmed RNase-free RNAse buffer), the sections are washed in 0.5×SSC for 10 minutes at room temperature. The sections are dehydrated in 70%, 95%, 100% ethanol, 2 minutes each.

B. Pretreatment of Paraffin-Embedded Sections

The slides are deparaffinized, placed in SQ H₂O, and rinsed twice in 2×SSC at room temperature, for 5 minutes each time. The sections are deproteinated in 20 μg/ml proteinase K (500 μl of 10 mg/ml in 250 ml RNase-free RNase buffer; 37 EC, 15 minutes)—human embryo, or 8× proteinase K (100 μl in 250 ml Rnase buffer, 37 EC, 30 minutes)—formalin tissues. Subsequent rinsing in 0.5×SSC and dehydration are performed as described above.

C. Prehybridization

The slides are laid out in a plastic box lined with Box buffer (4×SSC, 50% formamide)—saturated filter paper.

D. Hybridization

1.0×10⁶ cpm probe and 1.0 μl tRNA (50 mg/ml stock) per slide are heated at 95 EC for 3 minutes. The slides are cooled on ice, and 48 μl hybridization buffer are added per slide. After vortexing, 50 μl ³³P mix are added to 50 μl prehybridization on slide. The slides are incubated overnight at 55 EC.

E. Washes

Washing is done 2×10 minutes with 2×SSC, EDTA at room temperature (400 ml 20×SSC+16 ml 0.25M EDTA, V_{f}=4L), followed by RNaseA treatment at 37 EC for 30 minutes (500 μl of 10 mg/ml in 250 ml Rnase buffer=20 μg/ml). The slides are washed 2×10 minutes with 2×SSC, EDTA at room temperature. The stringency wash conditions can be as follows: 2 hours at 55 EC, 0.1×SSC, EDTA (20 ml 20×SSC+16 ml EDTA, V_{f}=4L).

F. Oligonucleotides

In situ analysis is performed on a variety of DNA sequences disclosed herein. The oligonucleotides employed for these analyses is obtained so as to be complementary to the nucleic acids (or the complements thereof) as shown in the accompanying figures.

In Situ Hybridization for Defensin Alpha 5. PCR primers were designed to amplify a 318 bp fragment of DEFA5 spanning from nt 55-372 of NM_{—}021010 (upper-5′ catcccttgctgccattct and lower-5′ gaccttgaactgaatcttgc). Primers included extensions encoding 27-nucleotide T7 or T3 RNA polymerase initiation sites to allow in vitro transcription of sense or antisense probes, respectively, from the amplified products. Endoscopic biopsies were fixed in 10% neutral buffered formalin and paraffin-embedded. Sections 5 μm thick were deparaffinized, deproteinated in 10 ug/ml Proteinase K (Amresco) for 45 minutes at 37° C., and further processed for in situ hybridization as previously described. (Jubb et. al. Methods Mol. Biol. 2006; 326:255-264) ³³P-UTP labeled sense and antisense probes were hybridized to the sections at 55° C. overnight. Unhybridized probe was removed by incubation in 20 μg/ml RNase A for 30 min at 37° C., followed by a high stringency wash at 55° C. in 0.1×SSC for 2 hours and dehydration through graded ethanols. The slides were dipped in NTB nuclear track emulsion (Eastman Kodak), exposed in sealed plastic slide boxes containing desiccant for 4 weeks at 4° C., developed and counterstained with hematoxylin and eosin.

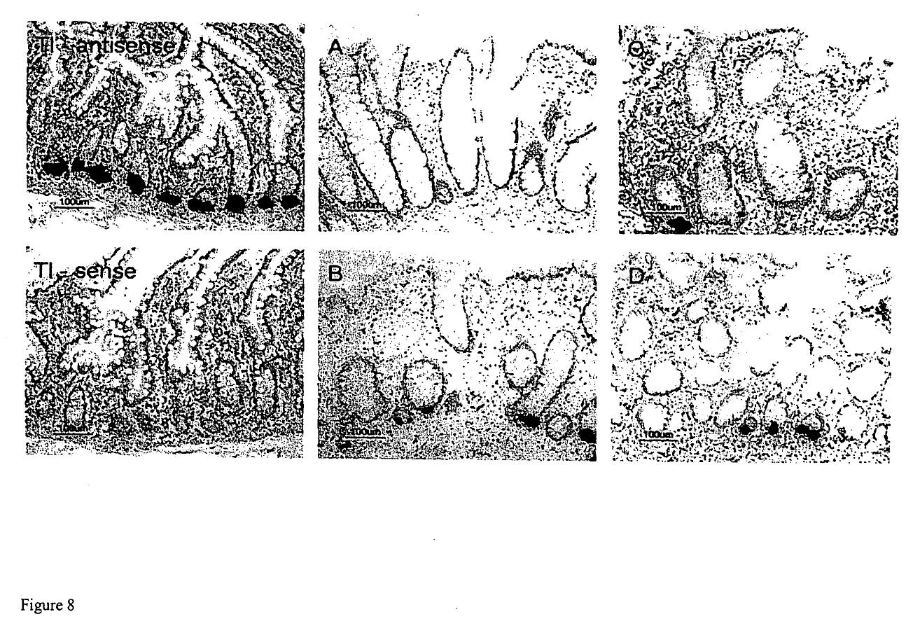

FIG. 8 shows the in-situ hybridization of the terminal ileal biopsies for DEFA5 showed strong hybridization in the basal crypts consistent with Paneth cell location. In the upper panel terminal ileum (TI), the antisense probe shows strong hybridization in the basal crypts consistent with Paneth cell location. In the lower panel terminal ileum (TI), no significant hybridization was observed with sense control probe. Panel A shows the sigmoid colon biopsy of a non-inflamed control patient. Panels B, C, & D show strong, multifocal hybridization in the basal crypt region of UC sigmoid colon biopsies consistent with Paneth cell metaplasia. In the UC biopsies taken from the sigmoid colon strong, multifocal hybridization in the basal crypt region of these biopsies was observed and this would be consistent with Paneth cell metaplasia. This was not observed in the non-inflamed control biopsies.

In-situ hybridization of the terminal ileal biopsies for DEFA6. Terminal ileum immunohistochemistry showed positive staining in the basal crypts consistent with Paneth cell location (data not shown). No significant staining was observed in the non-inflamed control patients (data not shown). Strong, multifocal staining in the basal crypt region of UC sigmoid colon biopsies consistent with Paneth cell metaplasia (data not shown). Immunohistochemistry for DEFA6 confirmed that in the sigmoid colon UC biopsies, staining was observed in the basal crypt region of these biopsies consistent with Paneth cell metaplasia. Again, this was not observed in the non-inflamed control biopsies (data not shown).

### Example 5

### Immunohistochemistry for Rabbit Anti-Human Lysozyme and Rabbit Anti-Human Defensin Alpha 6

Formalin fixed paraffin embedded tissue sections were rehydrated prior to quenching of endogenous peroxidase activity (KPL, Gaithersburg, Md.) and blocking of avidin and biotin (Vector, Burlingame, Calif.). Sections were blocked for 30 minutes with 10% normal goat serum in PBS with 3% BSA. Tissue sections were then incubated with primary antibodies for 60 minutes at room temperature, biotinylated secondary antibodies for 30 min, and incubated in ABC reagent (Vector, Burlingame, Calif.) for 30 minutes followed by a 5 minute incubation in metal enhanced DAB (Pierce, Rockford, Ill.). The sections were then counterstained with Mayer's hematoxylin. Primary antibodies used were rabbit anti-human lysozyme at 5.0 μg/ml (Dako, Carpinteria, Calif.) and rabbit anti-human DEFA6 at 5.0 μg/ml (Alpha Diagnostics, SanAntonio, Tex.). Secondary antibody used was biotinylated goat anti-rabbit IgG at 7.5 μg/ml (Vector, Burlingame, Calif.). DEFA6 alpha staining required pre-treatment with Target Retrieval High pH (Dako, Carpenteria, Calif.) at 99 C for 20 minutes, lysozyme staining did not require pretreatment. All other steps were performed at room temperature. Immunohistochemistry for DEFA6 confirmed that in the sigmoid colon UC biopsies, staining was observed in the basal crypt region of these biopsies consistent with Paneth cell metaplasia. Again, this was not observed in the control biopsies.

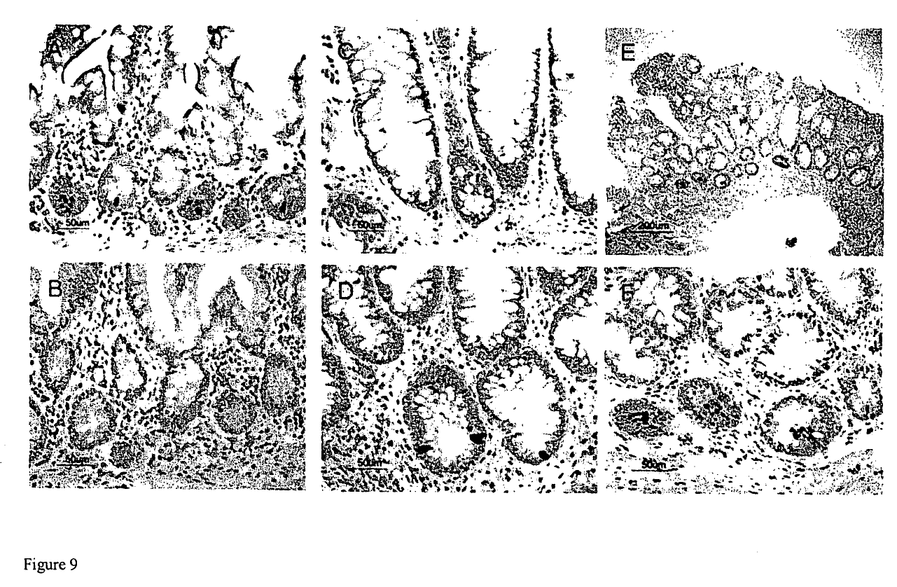

FIG. 9 shows the results for DefA6 in which (A) the terminal ileal immunohistochemistry shows positive staining in the basal crypts consistent with Paneth cell location. In B & C, no significant staining was observed in the non-inflamed control patients. In D, E & F, strong, multifocal staining in the basal crypt region of UC sigmoid colon biopsies consistent with Paneth cell metaplasia.

### Example 6

### Preparation of Antibodies that Bind Ihh Polypeptide, DefA5 Polypeptide or DefA6 Polypeptide

Techniques for producing monoclonal antibodies are known in the art and are described, for instance, in Goding, supra. Immunogens that may be employed include purified Ihh, DefA5 or DefA6 polypeptides, fusion proteins containing Ihh, DefA5 or DefA6 polypeptides, and cells expressing recombinant Ihh, DefA5 or DefA6 polypeptides on the cell surface. Selection of the immunogen can be made by the skilled artisan without undue experimentation.

Mice, such as Balb/c, are immunized with the above immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-Ihh, anti-DefA5 or anti-DefA6 antibodies.

After a suitable antibody titer has been detected, the animals “positive” for antibodies can be injected with a final intravenous injection of Ihh, DefA5 or DefA6 polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells are screened in, for example, an ELISA for reactivity against Ihh, DefA5 or DefA6 polypeptide. Determination of “positive” hybridoma cells secreting the desired monoclonal antibodies against Ihh, DefA5 or DefA6 polypeptide is within the skill in the art.

The positive hybridoma cells can be injected intraperitoneally into syngeneic Balb/c mice to produce ascites containing the anti-Ihh, anti-DefA5 or anti-DefA6 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 7

### Microarray Analysis to Detect Upregulation of DefA5 and DefA6 Gene Expression in Gastrointestinal tissue

Microarray analysis was used to find genes that are overexpressed in CD as compared to normal bowel tissue. For this study, sixty seven patients with CD and thirty-one control patients undergoing colonoscopy were recruited. Patient symptoms were evaluated at the time of colonoscopy using the simple clinical colitis activity index (SCCAI). (Walmsley et al., Gut. 1998; 43:29-32). Quiescent disease showing no histological inflammation was defined as a SCCAI of 2 or less. Active disease with histologially acute or chronic inflammation was defined as a SCCAI of greater than 2. The severity of the CD itself was determined by the criteria of Leonard-Jones. (Lennard-Jones Scand. J. Gastroent. 1989; 170:2-6). The CD patients provided well phenotyped biopsies for analysis of inflammatory pathways of CD at the molecular level, thus identifying novel candidate genes and potential pathways for therapeutic intervention. Paired biopsies were taken from each anatomical location.

All biopsies were stored at −70° C. until ready for RNA isolation. The biopsies were homogenized in 600 μl of RLT buffer (+BME) and RNA was isolated using Qiagen™ Rneasy Mini columns (Qiagen) with on-column DNase treatment following the manufacturer's guidelines. Following RNA isolation, RNA was quantitated using RiboGreen™ (Molecular Probes) following the manufacturer's guidelines and checked on agarose gels for integrity. Appropriate amounts of RNA were labeled for microarray analysis and samples were run on proprietary Genentech microarray and Affymetrics™ microarrays. Genes were compared whose expression was upregulated in UC tissue vs normal bowel, matching biopsies from normal bowel and CD tissue from the same patient. The results of this experiment showed that the nucleic acid shown as SEQ ID NO:3 (DEFA5) is differentially expressed in UC tissue in comparison to normal tissue, and the nucleic acid shown as SEQ ID NO:5 (DEFA6) is differentially expressed in CD and UC tissue in comparison to normal tissue. These genes demonstrated a minimum 1.5 fold difference in expression and also acceptable probe hybridization strength was observed. More specifically, SEQ ID NOS:3 and 5 represent polynucleotides and their encoded polypeptide which are significantly up-regulated/overexpressed in CD and/or UC.

### Example 8

### Characterisation of Distinct Intestinal Gene Expression Profiles in Ulcerative Colitis by Microarray Analysis

Microarray analysis allows a comprehensive picture of gene expression at the cellular level. The aim of this study was to investigate differential intestinal gene expression in patients with ulcerative colitis (UC) and controls.

Methods: 67 UC and 31 control subjects-23 normal and 8 inflamed non-inflammatory bowel disease patients were studied. Paired endoscopic biopsies were taken from 5 specific anatomical locations for RNA extraction and histology. 41058 expression sequence tags were analyzed in 215 biopsies using the Agilent platform. Confirmation of results was undertaken by real time PCR and immunohistochemistry. Results: In healthy control biopsies, cluster analysis showed differences in gene expression between the right and left colon. (χ²=25.1, p<0.0001). When all UC biopsies and control biopsies were compared, 143 sequences had a fold change of >1.5 in the UC biopsies (0.01>p>10⁻⁴⁵) and 54 sequences had a fold change of <−1.5 (0.01>p>10⁻²). Differentially upregulated in UC genes included the alpha defensins, DEFA5&6 (p=0.00003 and p=6.95×10⁻⁷ respectively). Increased DEFA5&6 expression was further characterized to Paneth cell metaplasia by immunohistochemistry and in-situ hybridization. The aim of the current study was to use microarray gene expression analysis to investigate genome wide expression in endoscopic mucosal biopsies of patients with UC and controls. In order to resolve previous inconsistencies and to further delineate inflammatory pathways in UC, substantially more patients and biopsies were included than in previous studies.

### Materials and Methods

Patients and Controls. Sixty seven patients with UC and 31 control patients who were undergoing colonoscopy were recruited. Their demographics are shown in Table 9.

TABLE 9

| | UC |
|---|---|
| Number of patients | 67 |
| Male/Female | 33/34 |
| Median age at diagnosis (years) | 37 |
| Median duration of follow up (years) | 7.8 |
| Disease Group | |
| New Diagnosis (1) | 8 |
| Quiescent disease (2) | 41 |
| Active disease (3) | 18 |
| Disease extent at time of Endoscopy | |
| Proctitis | 15 |
| L sided colitis | 27 |
| Extensive colitis | 25 |
| Current Smoker | 6 |
| Family history of IBD | 5 |
| 5 ASA Therapy | 40 |
| Corticosteroid therapy | 10 |
| Immunosuppressant therapy | 11 |
| (AZA, 6MP, MTX, MMF) | |

Sixty seven patients with UC and 31 control patients who were undergoing colonoscopy were recruited (Table 9). All UC patients attended the clinic at the Western General Hospital, Edinburgh and the diagnosis of UC adhered to the criteria of Lennard-Jones. (Lennard-Jones JE. Scand J Gastroenterol Suppl 1989; 170:2-6) Phenotypic data were collected by interview and case-note review and comprised of demographics, date of diagnosis, disease location, disease behavior, progression, extra-intestinal manifestations, surgical operations, current medication, smoking history, joint symptoms, family history and ethnicity. At the time of colonoscopy patients symptoms were evaluated using the simple clinical colitis activity index (SCCAI). (Walmsley et. al. Gut. 1998; 43:29-32)

Patients were recorded as having a ‘new diagnosis’ of UC if the colonoscopy took place at the time of their index presentation and they had had less than 24 hours of oral/IV therapy. Quiescent disease was defined as a SCCAI of 2 or less and histology showing no inflammation or mild chronic inflammation and active disease was defined as a SCCAI of greater than 2 and histology showing acute or chronic inflammation.

Eleven of the controls were male, 20 were female with a median age of 43 at the time of endoscopy. Six of the controls had normal colonoscopies for colon cancer screening, 9 controls had symptoms consistent with irritable bowel syndrome and had a normal colonoscopic investigation and 7 patients had a colonoscopy for another indication and histologically normal biopsies were obtained. Eight control patients had abnormal inflamed colonic biopsies (1 pseudomembranous colitis, 1 diverticulitis, 1 amoebiasis, 2 microscopic colitis, 1 eosoinophilic infiltrate, 2 scattered lymphoid aggregates and a history of gastroenteritis). Written informed consent was obtained from all patients. Lothian Local Research Ethics Committee approved the study protocol: REC 04/S1103/22.

Biopsy Collection. Anatomical location was confirmed by an experienced operator, distance of endoscope insertion and endoscope configuration using a Scope Guide™. Paired biopsies were taken from each anatomical location. One biopsy was sent for histological examination and the other was snap frozen in liquid nitrogen for RNA extraction. Each biopsy was graded histologically, by an experienced gastrointestinal pathologist as having no evidence on inflammation, biopsies with evidence of chronic inflammation and predominately chronic inflammatory cell infiltrate or simply those with acute inflammation and an acute inflammatory cell infiltrate. One hundred and thirty nine paired UC biopsies and 76 paired control biopsies were collected. The number of paired biopsies in UC patients and controls from each anatomical location are shown in Table 10.

TABLE 10

| | UC (n = 67) | Controls (n = 31) |
|---|---|---|
| Total number of paired biopsies | 139 | 76 |
| Terminal Ileum | 4 | 6 |
| Ascending colon | 33 | 17 |
| Descending colon | 35 | 23 |
| Sigmoid colon biopsies. | 57 | 27 |
| Removed from analysis | 10 | 3 |

RNA Isolation. The biopsies weighed between 0.2 mg and 16.5 mg with a median weight of 5.5 mg. Total RNA was extracted from each biopsy using the micro total RNA isolation from animal tissues protocol (Qiagen, Valencia, Calif.), according to the manufacturer's instructions. To evaluate purity and integrity 1 μL of total RNA was assessed each sample with the Agilent technologies 2100 bioanalyzer using the Pico LabChip reagent set (Agilent Technologies, Palo Alto, Calif.).

Microarray Analysis. 1 μg of total RNA was amplified using the Low RNA Input Fluorescent Linear Amplification protocol (Agilent Technologies, Palo Alto, Calif.). A T7 RNA polymerase single round of linear amplification was carried out to incorporate Cyanine-3 and Cyanine-5 label into cRNA. The cRNA was purified using the RNeasy Mini Kit (Qiagen, Valencia, Calif.). 1 μl of CRNA was quantified using the NanoDrop ND-1000 spectrophotometer (NanoDrop Technologies, Wilmington, Del.).

750 ng of Universal Human Reference (Stratagene, La Jolla, Calif.) cRNA labeled with Cyanine-3 and 750 ng of the test sample cRNA labeled with Cyanine-5 were fragmented for 30 minutes at 60° C. before loading onto Agilent Whole Human Genome microarrays (Agilent technologies, Palo Alto, Calif.). The samples were hybridized for 18 hours at 60° C. with constant rotation. Microarrays were washed, dried and scanned on the Agilent scanner according to the manufacturer's protocol (Agilent technologies, Palo Alto, Calif.). Microarray image files were analyzed using Agilent's Feature Extraction software version 7.5 (Agilent Technologies, Palo Alto, Calif.). The distribution of log intensities for each sample was plotted and outlier samples (i.e. greater than 2 standard deviations from the mean) were excluded from analysis. 10 UC samples and 3 control samples were designated as outliers using these criteria.

Analysis of expression in UC and control biopsies. Using unsupervised hierarchical clustering we were unable to differentiate between biopsies from UC patients and controls patients. In addition no clustering based on the inflammation status of the biopsies was observed. The only clustering that was observed was with biopsies from the terminal ileum where both UC and control biopsies clustered together. When all of the UC biopsies (129) and control biopsies (73) were compared, 143 sequences had a fold change of greater than 1.5 in the UC biopsies (0.01>p>10⁻⁴⁵) and 54 sequences had a fold change of less than 1.5 (0.01>p>10⁻²⁰) (data not shown).

Notably upregulation was observed for genes corresponding to the alpha defensins: alpha 5 (DEFA5) (FC+3.25, p=0.00003) and alpha 6 (DEFA6) (FC+2.18, p=6.95×10⁻⁷). The differential gene expression of DEFA5 and DEFA6 a number of candidate genes across more than one experiment is shown in Table 11, which shows fold changes and p values in four different experiments. The number of biopsies analyzed in each experiment is shown in brackets. Significant consistent changes in expression across more than one experiment were observed.

TABLE 11

| Genes Analyzed | | p value |
|---|---|---|
| | All UC (129 biopsies) v controls | |
| | (73 biopsies) Fold change | |
| Def alpha 5 | +3.25 | 0.00003 |
| Def alpha 6 | +2.18 | 6.95 × 10⁻⁷ |
| | Non-inflamed UC sigmoid (22) v | |
| | non-inflamed control | |
| | sigmoid (18) Fold change | |
| Def alpha 5 | +1.02 | 0.89 |
| Def alpha 6 | −1.09 | 0.34 |
| | Inflamed UC sigmoid (35) v | |
| | inflamed control | |
| | sigmoid (8) Fold change | |
| Def alpha 5 | +7.27 | 6.3 × 10⁻³⁰ |
| Def alpha 6 | +4.41 | 9.7 × 10⁻⁹ |
| | Inflamed UC sigmoid (35) v | |
| | non-inflamed sigmoid | |
| | UC (22) Fold change | |
| Def alpha 5 | +8.44 | <10⁻⁴⁵ |
| Def alpha 6 | +6.72 | 4.16 × 10⁻¹⁹ |

Analysis of expression in sigmoid colon biopsies in patients with quiescent UC and non-inflamed control biopsies. To compare expression in biopsies without an acute inflammatory signal and to remove the effect of anatomical variation, 22 biopsies from the sigmoid colon with no histological evidence of inflammation from patients with UC were compared to 18 histologically normal control sigmoid colon biopsies. 102 sequences had a fold change greater than 1.5 (0.01>p>4.77×10⁻¹³) and 84 sequences had a fold change of less than 1.5 (0.01>p>1.8×10⁻²¹) (data not shown).

Inflamed versus non-inflamed UC sigmoid colon biopsies. When expression signals were compared between 35 histologically inflamed and 22 non-inflamed sigmoid colon UC biopsies 700 sequences had a fold change of greater than 1.5 (0.01>p>×10⁻⁴⁵) and 518 sequences (0.01>p>1×10⁴⁵) had a fold change of less than 1.5 in the inflamed biopsies (data not shown). The upregulated genes included DEFA5 (FC+8.44, p=<10-45) and DEFA6 (FC+6.72, p=4.16×10⁻¹⁹).

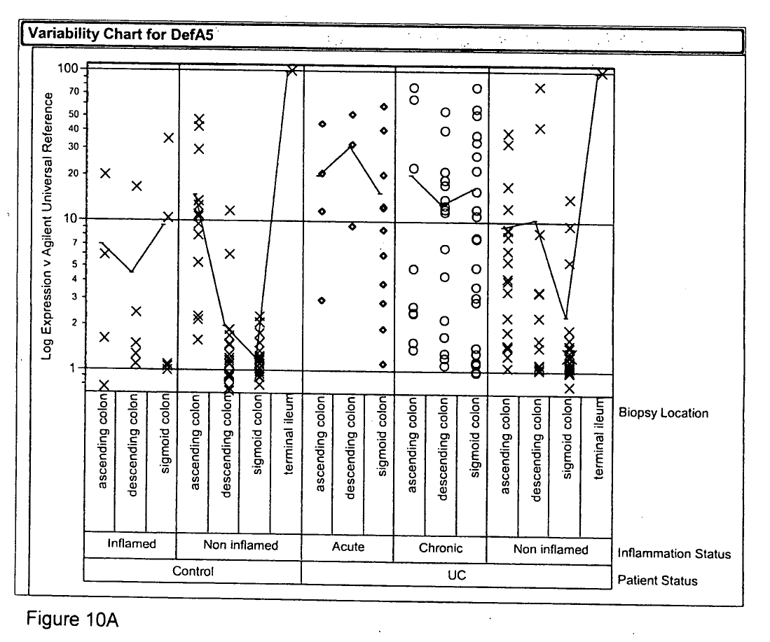

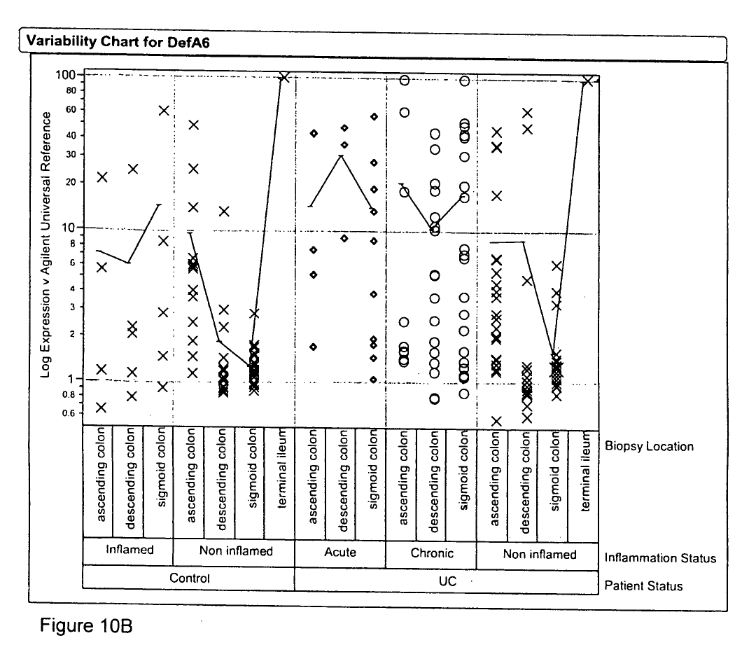

Analysis of Specific Gene Families-Alpha Defensins 5 and 6. Expression of a number of genes of interest was further analysed, taking into consideration anatomical location and degree of inflammation in the UC samples. When DEFA5 and DEFA6 were analysed expression in the normal controls and the non-inflamed UC biopsies was similar across the different anatomical locations with there being high expression in the terminal ileum, and expression decreasing as the biopsy location became more distal in the colon (FIG. 10).

In FIG. 10, the expression of each array sample is plotted against the Agilent universal reference. Each endoscopic biopsy has been separated by patient status, biopsy inflammation status and anatomical location. The mean expression levels for each anatomical location are linked in blue. High alpha defensin 5 (panel A) and 6 (B) (DEFA5 and DEFA6) expression levels are seen in the terminal ileum of the controls and the non inflamed UC samples. The expression in these 2 groups decreased the more distally in the colon the biopsies were retrieved from. In the acute and chronically inflamed UC samples and to a lesser extent in the inflamed control samples there was a marked increase in DEFA5 and DEFA6 expression throughout the ascending, descending and sigmoid colon-sigmoid colon inflamed v non-inflamed UC samples (FC+8.44, p=<10⁻⁴⁵) for DEFA5, (FC+6.72, p=4.16×10⁻¹⁹) for DEFA6.

In the acute and chronically inflamed UC biopsies there was marked upregulation of DEFA5 and DEFA6 expression throughout the ascending, descending and sigmoid colon (Table 11).

Although the foregoing invention has been described in some detail by way of illustration and example for purposes of clarity of understanding, the descriptions and examples should not be construed as limiting the scope of the invention. The disclosures of all patent and scientific literatures cited herein are expressly incorporated in their entirety by reference.

What is claimed is:

1. A method of diagnosing the presence of an inflammatory bowel disease (IBD) in a mammalian subject, comprising
(a) determining that a level of expression of a nucleic acid encoding a polypeptide shown as SEQ ID NO:2 in a test sample obtained from said subject is lower relative to a level of expression in a control, wherein said lower level of expression is indicative of the presence of an IBD in the subject from which the test sample was obtained.

2. A method of diagnosing the presence of an inflammatory bowel disease (IBD) in a mammalian subject, comprising
(a) determining that a level of expression of a nucleic acid encoding a polypeptide shown as SEQ ID NO:4 and/or a polypeptide shown as SEQ ID NO:6 in a test sample obtained from said subject is higher relative to a level of expression in a control, wherein said higher level of expression is indicative of the presence of an IBD in the subject from which the test sample was obtained.

3. A method of diagnosing the presence of an inflammatory bowel disease (IBD) in a mammalian subject, comprising
(a) determining that a level of expression of a nucleic acid encoding a polypeptide shown as SEQ ID NO:2 in a first test sample obtained from said subject is lower relative to the level of expression in a first control, wherein said lower level of expression is indicative of the presence of an IBD in the subject from which the first test sample was obtained; and
(b) determining that a level of expression of a nucleic acid encoding a polypeptide shown as SEQ ID NO:4 and/or a polypeptide shown as SEQ ID NO:6 in a second test sample obtained from said subject is higher relative to the level of expression in a second control, wherein said higher level of expression is indicative of the presence of an IBD in the subject from which the second test sample was obtained.

4. The method of claim 1, 2 or 3 wherein said mammalian subject is a human patient.

5. The method of claim 4 wherein evidence of said expression level is obtained by a method of gene expression profiling.

6. The method of claim 5 wherein said method is a PCR-based method.

7. The method of claim 5 wherein said expression levels are normalized relative to the expression levels of one or more reference genes, or their expression products.

8. The method of claim 1, 2 or 3 further comprising the step of creating a report summarizing said IBD detection.

9. The method of claim 1, 2 or 3, wherein said IBD is ulcerative colitis.

10. The method of claim 1, 2 or 3, wherein said IBD is Crohn's disease.

11. The method of claim 1, 2 or 3, wherein said IBD is ulcerative colitis and Crohn's disease.

12. The method of claim 1, 2 or 3, wherein said test sample is from a colonic tissue biopsy.

13. The method of claim 12, wherein said biopsy is from a tissue selected from the group consisting of the terminal ileum, the ascending colon, the descending colon, and the sigmoid colon.

14. The method of claim 12, wherein said biopsy is from an inflamed colonic area.

15. The method of claim 12, wherein said biopsy is from a non-inflamed colonic area.

16. The method of claim 1, 2 or 3, wherein said determining step (a) and/or (b) is indicative of a recurrence of an IBD in said mammalian subject, and wherein said mammalian subject was previously diagnosed with an IBD and treated for said previously diagnosed IBD.

17. The method of claim 15, wherein said treatment comprised surgery.

18. The method of claim 1 or 2, wherein said determining step (a) and/or (b) is indicative of a flare-up of said IBD in said mammalian subject.

19. The method of claim 3, wherein said first test sample and said second test sample are the same.

20. The method of claim 3, wherein said first control and said second control are the same.

21. A method of treating an inflammatory bowel disease (IBD) in a mammalian subject in need thereof, the method comprising the steps of
(a) determining that a level of expression of a nucleic acid encoding a polypeptide shown as SEQ ID NO:2 in a test sample obtained from said subject is lower relative to a level of expression in a control, wherein said lower level of expression is indicative of the presence of an IBD in the subject from which the test sample was obtained; and
(b) administering to said subject an effective amount of an IBD therapeutic agent.

22. A method of treating an inflammatory bowel disease (IBD) in a mammalian subject in need thereof, the method comprising the steps of
(a) determining that a level of expression of a nucleic acid encoding a polypeptide shown as SEQ ID NO:4 and/or a polypeptide shown as SEQ ID NO:6 in a test sample obtained from said subject is higher relative to a level of expression in a control, wherein said higher level of expression is indicative of the presence of an IBD in the subject from which the test sample was obtained; and
(b) administering to said subject an effective amount of an IBD therapeutic agent.

23. A method of treating an inflammatory bowel disease (IBD) in a mammalian subject, comprising
(a) determining that a level of expression of a nucleic acid encoding a polypeptide shown as SEQ ID NO:2 in a first test sample obtained from said subject is lower relative to a level of expression in a first control, wherein said lower level of expression is indicative of the presence of an IBD in the subject from which the first test sample was obtained;
(b) determining that a level of expression of a nucleic acid encoding a polypeptide shown as SEQ ID NO:4 and/or a polypeptide shown as SEQ ID NO:6 in a second test sample obtained from said subject is higher relative to a level of expression in a second control, wherein said higher level of expression is indicative of the presence of an IBD in the subject from which the second test sample was obtained; and
(c) administering to said subject an effective amount of an IBD therapeutic agent.

24. The method of claim 21, 22 or 23 wherein said mammalian subject is a human patient.

25. The method of claim 24 wherein evidence of said expression level is obtained by a method of gene expression profiling.

26. The method of claim 25 wherein said method is a PCR-based method.

27. The method of claim 25 wherein said expression levels are normalized relative to the expression levels of one or more reference genes, or their expression products.

28. The method of claim 21, 22 or 23 further comprising the step of creating a report summarizing said IBD detection.

29. The method of claim 21, 22 or 23, wherein said IBD is ulcerative colitis.

30. The method of claim 21, 22 or 23, wherein said IBD is Crohn's disease.

31. The method of claim 21, 22 or 23, wherein said IBD is ulcerative colitis and Crohn's disease.

32. The method of claim 21, 22 or 23, wherein said test sample is from a colonic tissue biopsy.

33. The method of claim 32, wherein said biopsy is from a tissue selected from the group consisting of the terminal ileum, the ascending colon, the descending colon, and the sigmoid colon.

34. The method of claim 32, wherein said biopsy is from an inflamed colonic area.

35. The method of claim 32, wherein said biopsy is from a non-inflamed colonic area.

36. The method of claim 21, 22 or 23, wherein said determining step is indicative of a recurrence of an IBD in said mammalian subject, and wherein said mammalian subject was previously diagnosed with an IBD and treated for said previously diagnosed IBD.

37. The method of claim 36, wherein said treatment comprised surgery.

38. The method of claim 21, 22 or 23, wherein said determining step is indicative of a flare-up of said IBD in said mammalian subject.

39. The method of claim 21, 22 or 23, wherein said IBD therapeutic agent is an aminosalicylate.

40. The method of claim 21, 22 or 23, wherein said IBD therapeutic agent is a corticosteroid.

41. The method of claim 21, 22 or 23, wherein said IBD therapeutic agent is an immunosuppressive agent.

42. The method of claim 23, wherein said first test sample and said second test sample are the same.

43. The method of claim 23, wherein said first control and said second control are the same.
